(19) United States
(12) Patent Application Publication
(10) Pub. No.: US 2024/0254537 A1
(43) Pub. Date: Aug. 1, 2024

(54) POOLED OPTICAL SCREENING AND TRANSCRIPTIONAL MEASUREMENTS OF CELLS COMPRISING BARCODED GENETIC PERTURBATIONS

(71) Applicant: Insitro, Inc., South San Francisco, CA (US)

(72) Inventors: Max R. SALICK, San Francisco, CA (US); Srinivasan SIVANANDAN, South San Francisco, CA (US); Cynthia HAO, Saratoga, CA (US); Eric LUBECK, San Francisco, CA (US); Ajamete KAYKAS, San Francisco, CA (US); Ci CHU, Palo Alto, CA (US)

(73) Assignee: Insitro, Inc., South San Francisco, CA (US)

(21) Appl. No.: 18/113,481

(22) Filed: Feb. 23, 2023

Related U.S. Application Data
(60) Provisional application No. 63/313,189, filed on Feb. 23, 2022.

(51) Int. Cl. C12Q 1/6806; C12Q 1/6816; C12Q 1/6827; C12Q 1/6841; C12Q 1/6874

(52) U.S. Cl. CPC C12Q 1/6806; C12Q 1/6816; C12Q 1/6827; C12Q 1/6841; C12Q 1/6874; C12Q 2600/156; C12Q 2600/158

(57) ABSTRACT

The present disclosure relates to methods of pooled optical screening of genetically barcoded cells comprising genetic perturbations, and simultaneous transcriptional measurements.

### CROSS-REFERENCE TO RELATED APPLICATIONS

This application claims priority benefit to U.S. Provisional Application No. 63/313,189, filed on Feb. 23, 2022, the contents of which are hereby incorporated by reference in their entirety for all purposes.

### FIELD OF THE INVENTION

The present disclosure relates in some aspects to methods of pooled optical screening of genetically barcoded cells comprising genetic perturbations, and simultaneous transcriptional measurements

### BACKGROUND OF THE INVENTION

The examination of genetic perturbations is a critical component of modern biological research that allows scientists to understand disease, map biological pathways, and identify targets for drug discovery. While most studies focus on individual genetic knockouts at one time, new screening technologies have recently emerged in which several genes, or even the entire genome, distributed across a population of cells, receive targeted mutations to probe the effects of various perturbations on a given biological system. Current industry standard methods involve sorting on a targeted biomarker, often using reporter cell lines or antibody stains, e.g., Clustered Regularly Interspaced Short Palindromic Repeats (CRISPR) screening. Other methodologies merge the sequencing of barcodes with single cell transcriptomics (“Perturb-Seq”; Dixit A, et al., 2016. Cell 167(7), 1853-1866, and CRISPR-droplet sequencing (“CROP-Seq”); Datlinger P, et al., 2017. Nat Methods 14(3), 297-301). While these techniques are effective at mapping the effect of genetic perturbations on biological pathways, the high cost of these screening strategies only permits the examination of a small selection of genes, while losing much of the cells' morphological information in the process. More recently, an imaging-based method has emerged that reduces much of this cost and allows for the matching of perturbations to optical phenotypes (see, e.g., Feldman D., et al., 2019. Cell 179, 787-799), though this method does not allow for simultaneous transcriptional measurements, such as those determined using fluorescence in situ hybridization (see, e.g., Wang X., et al., 2018. Science 361(6400)). Improved methods of assessing cells are needed in the art.

All references cited herein, including patent applications, patent publications, and scientific literature, are herein incorporated by reference in their entirety, as if each individual reference were specifically and individually indicated to be incorporated by reference.

### SUMMARY OF THE INVENTION

Disclosed herein are methods, systems, electronic devices, non-transitory storage media, and apparatuses directed to providing a pooled optical screening platform for genetically engineered barcoded cells with a corresponding mRNA readout. The methods can be applied to evaluate morphological and protein phenotypes, transcriptional information, and genetic perturbation data into a single pooled assay. In some embodiments, the methods comprise a set of techniques, including genetic engineering, fluorescence in situ hybridization (FISH) (including one or more of RNA FISH, RNA foci FISH, and immunoFISH), pooled optical screening (POSH), and, optionally, various proteomic and morphological analyses to study optical phenotypes and transcriptional measures without disrupting cellular morphology and preserving the cells to allow for downstream measurement of barcoded perturbations.

The platform provides numerous practical applications related to the studying of cellular processes. For example, the methods provide a single combined assay comprising a single, unified workflow to include genetic perturbations, live cell readouts, transcriptional readouts, and protein/morphological/imaging readouts all within the same cell, thereby providing high throughput, multi-omic datasets. The techniques also allow for a more tunable design of RNA probes, which expands the screening capacity to include splicing effects and other transcriptional events that might be important for a given biological process or disease. Additionally, the method does not require use of an instrument to perform current next-generation sequencing technologies, which substantially reduces the cost.

In some aspects, provided herein are methods, comprising: (a) providing a plurality of cells, wherein the plurality of cells comprise at least one cell comprising at least one genetic perturbation, wherein said at least one cell comprising the at least one genetic perturbation comprises a barcode sequence associated with the genetic perturbation; (b) performing at least one round of fluorescence in situ hybridization (FISH); (c) performing pooled optical screening in human cells (POSH), comprising amplifying the barcode sequence in the at least one cell and sequencing the barcode sequence in situ.

In some embodiments, the method further comprises analyzing the phenotype of the at least one cell; wherein analyzing the phenotype comprises at least one assay selected from the group consisting of label-free imaging, high content imaging, calcium imaging, immunohistochemistry, cell morphology imaging, protein aggregation imaging, cell-cell interaction imaging, live cell imaging, and any other imaging-based assay modality. In some embodiments, the plurality of cells are selected from the group consisting of immortalized cancer cell lines, primary cells, primary tissue biopsies, and patient-derived cancer cells. In some embodiments, the plurality of cells are derived from induced pluripotent stem cells (iPSCs). In some embodiments, the method further comprises differentiating the iPSCs to derive the plurality of cells. In some embodiments, the iPSCs are differentiated into hepatic stellate cells.

In some embodiments, the at least one cell comprises a CRISPR system. In some embodiments, the CRISPR system is a CRISPR interference (CRISPRi) system. In some embodiments, the CRISPR system is a CRISPR activation (CRISPRa) system. In some embodiments, the at least one cell comprises and/or expresses a Cas protein. In some embodiments, the Cas protein is selected from the group consisting of a Cas9 protein, a Cas12a protein, and a Cas13 protein. In some embodiments, the Cas protein is a Cas9 protein.

In some embodiments, the at least one cell has been contacted with a gRNA to generate the at least one perturbation. In some embodiments, the gRNA is or contains the barcode sequence. In some embodiments, the barcode sequence comprises the gRNA sequence or a portion of the gRNA sequence. In some embodiments, the method further comprises contacting the plurality of cells with a gRNA to generate the at least one genetic perturbation. In some embodiments, the method further comprises contacting the plurality of cells with a gRNA library comprising a plurality of different gRNAs to generate a plurality of genetic perturbations comprising the at least one genetic perturbation. In some embodiments, the method further comprises synthesizing the gRNA library. In some embodiments, the method further comprises engineering the gRNA into a viral vector or the gRNA library into a library of viral vectors. In some embodiments, the viral vector or library of viral vectors encode a selectable marker, optionally wherein the selectable marker is an antibiotic resistance gene. In some embodiments, the method further comprises contacting the plurality of cells with the viral vector or library of viral vectors. In some embodiments, the gRNA is flanked by probe hybridization sequences in the vector. In some embodiments, the gRNA or the plurality of gRNAs hybridize(s) with one or more target sequences in the at least one cell. In some embodiments, the target sequence is a nucleic acid sequence that is complementary, or partially complementary, to the gRNA, or a portion thereof.

In some embodiments, the method further comprises fixing the plurality of cells on a surface prior to step (b). In some embodiments, fixing the plurality of cells comprises at least one of paraformaldehyde treatment and methanol treatment. In some embodiments, the method further comprises permeabilizing the plurality of cells. In some embodiments, permeabilizing the plurality of cells comprises at least one of ethanol treatment and treatment with a detergent. In some embodiments, permeabilizing the plurality of cells comprises treatment with a detergent, wherein the detergent is a Triton family detergent or a Tween family detergent.

In some embodiments, the at least one round of FISH comprises at least one round of RNA FISH, wherein each round of RNA FISH is uniquely associated with at least one mRNA transcript from the at least one cell. In some embodiments, the at least one round of RNA FISH comprises: (i) contacting a plurality of mRNA transcripts comprising the at least one mRNA transcript with a plurality of 3′ loop probes, wherein each 3′ loop probe comprises a first target hybridization sequence complementary to a first portion of an mRNA transcript of the plurality of mRNA transcripts; (ii) contacting the plurality of mRNA transcripts comprising the at least one mRNA transcript with a plurality of 5′ probes, wherein each 5′ probe comprises a second target hybridization sequence complementary to a second portion of the mRNA transcript of the plurality of mRNA transcripts, wherein each 5′ probe is capable of specifically hybridizing with a 3′ loop probe, wherein hybridization of a 5′ probe with a corresponding 3′ loop probe forms a loop in the 3′ loop probe; (iii) connecting the ends of the loop in each 3′ loop probe to form a plurality of circular probes; (iv) amplifying a plurality of target sequences using the circular probes as templates to form a plurality of DNA amplicons; and (v) detecting the DNA amplicons by the at least one round of RNA FISH. In some embodiments, the at least one round of RNA FISH comprises: (i) contacting a plurality of mRNA transcripts comprising the at least one mRNA transcript with a plurality of 5′ loop probes, wherein each 5′ loop probe comprises a first target hybridization sequence complementary to a first portion of an mRNA transcript of the plurality of mRNA transcripts; (ii) contacting the plurality of mRNA transcripts comprising the at least one mRNA transcript with a plurality of 3′ probes, wherein each 3′ probe comprises a second target hybridization sequence complementary to a second portion of the mRNA transcript of the plurality of mRNA transcripts, wherein each 3′ probe is capable of specifically hybridizing with a 5′ loop probe, wherein hybridization of a 3′ probe with a corresponding 5′ loop probe forms a loop in the 5′ loop probe; (iii) connecting the ends of the loop in each 5′ loop probe to form a plurality of circular probes; (iv) amplifying a plurality of target sequences using the circular probes as templates to form a plurality of DNA amplicons; and (v) detecting the DNA amplicons by the at least one round of RNA FISH. In some embodiments, the DNA amplicons each comprise a plurality of copies of their corresponding mRNA transcripts. In some embodiments, detecting the DNA amplicons comprises labeling the DNA amplicons with a fluorophore, an isotope, a mass tag, or a combination thereof. In some embodiments, detecting the DNA amplicons comprises hybridizing an adapter oligonucleotide to the DNA amplicon. In some embodiments, detecting the DNA amplicons comprises hybridizing a detection probe to the adapter oligonucleotide. In some embodiments, the detection probe comprises a fluorophore, an isotope, a mass tag, an oligonucleotide, or a combination thereof. In some embodiments, detecting the DNA amplicons comprises imaging the DNA amplicons. In some embodiments, imaging comprises recording a relative position in an image field. In some embodiments, the method comprises removing any unbound detection probes prior to detection. In some embodiments, the method further comprises removing the adapter oligonucleotide by contacting the adapter oligonucleotide with a toehold oligonucleotide capable of displacing each adapter oligonucleotide from each DNA amplicon. In some embodiments, the 3′ loop probe is a DNA molecule. In some embodiments, the 5′ probe is a DNA molecule. In some embodiments, the 5′ loop probe is a DNA molecule. In some embodiments, the 3′ probe is a DNA molecule. In some embodiments, the DNA amplicon is formed using rolling circle amplification (RCA). In some embodiments, the RCA comprises a linear RCA, a branched RCA, a dendritic RCA, or any combination thereof. In some embodiments, the DNA amplicon is formed using a Phi29 polymerase. In some embodiments, the connecting step (iii) comprises ligation selected from the group consisting of enzymatic ligation, chemical ligation, template dependent ligation, and/or template independent ligation. In some embodiments, the ligation is enzymatic ligation by a ligase. In some embodiments, the ligase is a T4 RNA ligase, a splintR ligase, a single stranded DNA ligase, or a T4 DNA ligase. In some embodiments, the ligase has a DNA-splinted DNA ligase activity. In some embodiments, the ends of the 3′ loop probe are ligated without gap filling prior to ligation. In some embodiments, the ends of the 5′ loop probe are ligated without gap filling prior to ligation. In some embodiments, the method further comprises fixing the DNA amplicons to a surface. In some embodiments, the fixing comprises treatment with bis-N-succinimidyl-(pentaethylene glycol) (bis-MHS-PEG).

In some embodiments, the at least one round of FISH comprises at least one round of RNA foci FISH. In some embodiments, the at least one round of RNA foci FISH comprises: (i) contacting RNA foci with a fluorescent oligonucleotide probe, wherein each fluorescent oligonucleotide probe comprises a target hybridization sequence complementary to a portion of a sequence in the RNA foci; and (ii) detecting the fluorescent oligonucleotide probe by imaging.

In some embodiments of the preceding methods, at least two rounds of RNA FISH are performed. In some embodiments, at least one round of RNA FISH, or each round of RNA FISH, is associated with an mRNA transcript of a regulatory gene from the at least one cell in addition to the at least one mRNA transcript of each round.

In some embodiments of the preceding methods, POSH comprises: (A) reverse transcribing the barcode sequence to form a reverse transcribed barcode sequence; (B) hybridizing at least one padlock probe to the reverse transcribed barcode sequence, wherein: the at least one padlock probe comprises a first barcode hybridization sequence and a second barcode hybridization sequence, the reverse transcribed barcode sequence comprises a first padlock probe hybridization sequence and a second padlock probe hybridization sequence flanking a target sequence, and the first barcode hybridization sequence hybridizes with first padlock probe hybridization sequence and the second barcode hybridization sequence hybridizes with second padlock probe hybridization sequence; and (C) connecting the ends of the at least one padlock probe to form a circular probe. In some embodiments, the method further comprises (D) forming a barcode amplicon using the circular probe as a template, wherein the barcode amplicon comprises a plurality of copies of the barcode sequence. In some embodiments, the at least one padlock probe is a DNA molecule. In some embodiments, the barcode amplicon is formed using RCA. In some embodiments, the RCA comprises a linear RCA, a branched RCA, a dendritic RCA, or any combination thereof. In some embodiments, the barcode amplicon is formed using a Phi29 polymerase. In some embodiments, the ends of the at least one padlock probe are connected by gap filling. In some embodiments, the connecting step comprises ligation selected from the group consisting of enzymatic ligation, chemical ligation, template dependent ligation, and/or template independent ligation. In some embodiments, the ligation is enzymatic ligation utilizing a ligase selected from the group consisting of a T4 RNA ligase, a splintR ligase, a single stranded DNA ligase, or a T4 DNA ligase. In some embodiments, the ends of the at least one padlock probe are ligated without gap filling prior to ligation. In some embodiments, the method further comprises fixing the reverse transcribed barcode sequence to a surface. In some embodiments, sequencing the barcode sequence in situ comprises sequencing by hybridization, sequencing by ligation, sequencing by synthesis, and/or sequencing by binding. In some embodiments, permeabilizing the plurality of cells is performed before performing the at least one round of RNA FISH, amplifying of the barcode sequence in the at least one cell, and sequencing of the barcode sequence in situ. In some embodiments, the method further comprises fixing the DNA amplicons in place after step (iv), and before step (v), of the at least one round of RNA FISH. In some embodiments, fixing the DNA amplicons comprises bis-MHS-PEG treatment. In some embodiments, fixing the DNA amplicons occurs before step (A) of POSH. In some embodiments, the method further comprises fixing the reverse transcribed barcode sequence in place after step (A) of POSH. In some embodiments, fixing the reverse transcribed barcode sequence comprises at least one of paraformaldehyde (PFA) and glutaraldehyde treatment. In some embodiments, fixing the reverse transcribed barcode sequence comprises both PFA and glutaraldehyde treatment. In some embodiments, fixing the reverse transcribed barcode sequence is performed after step (iv) of the at least one round of RNA FISH. In some embodiments, fixing the reverse transcribed barcode sequence is performed before step (v) of the at least one round of RNA FISH. In some embodiments, the barcode amplicon is formed after step (v) of the at least one round of RNA FISH. In some embodiments, analyzing the phenotype of the at least one cell is performed after step (D) of POSH. In some embodiments, analyzing the phenotype of the at least one cell is performed before step (D) of POSH. In some embodiments, the at least one round of RNA foci FISH is performed before step (A) of POSH. In some embodiments, step (i) of contacting RNA foci with the fluorescent oligonucleotide probe is carried out during an incubation period at a temperature of at least 50° C.

In some embodiments, the at least one round of FISH comprises at least one round of immunoFISH, wherein each round of immunoFISH is uniquely associated with at least one protein of interest from the at least one cell. In some embodiments, the at least one round of immunoFISH comprises: (i) contacting the at least one cell with a conjugate, the conjugate comprising an antibody which specifically binds the at least one protein of interest and a DNA oligonucleotide comprising a protein identification sequence uniquely associated with the protein of interest; (ii) contacting the conjugate with an adapter oligonucleotide, wherein the adapter oligonucleotide comprises a first adapter sequence complementary to a portion of the DNA oligonucleotide; (iii) contacting the adapter oligonucleotide with a fluorescent oligonucleotide capable of hybridizing with a second adapter sequence of the adapter oligonucleotide; (iv) imaging the at least one cell to detect the at least one fluorescent oligonucleotide. In some embodiments, the at least one round of immunoFISH comprises: (i) contacting the at least one cell with a first antibody which specifically binds the at least one protein of interest; (ii) contacting the antibody with a conjugate comprising an antibody which specifically binds the first antibody and a DNA oligonucleotide comprising a protein identification sequence uniquely associated with the protein of interest; (iii) contacting the conjugate with an adapter oligonucleotide, wherein the adapter oligonucleotide comprises a first adapter sequence complementary to a portion of the DNA oligonucleotide; (iv) contacting the adapter oligonucleotide with a fluorescent oligonucleotide capable of hybridizing with a second adapter sequence of the adapter oligonucleotide; (v) imaging the at least one cell to detect the at least one fluorescent oligonucleotide. In some embodiments, the conjugate is directly conjugated to the DNA oligonucleotide. In some embodiments, the conjugate is indirectly conjugated to the DNA oligonucleotide. In some embodiments, the antibody is streptavidin bound and the DNA oligonucleotide is biotinylated, wherein the conjugation is via streptavidin-biotin. In some embodiments, the method further comprises displacing the adapter oligonucleotide by contacting the adapter oligonucleotide with a toehold oligonucleotide capable of displacing each adapter oligonucleotide from each conjugate. In some embodiments, imaging comprises recording a position of the fluorescent oligonucleotide in an image field. In some embodiments, at least two rounds of immunoFISH are performed, wherein a second round of immunoFISH is performed after inactivating or displacing the fluorescent oligonucleotide. In some embodiments, each round of immunoFISH is associated with a regulatory protein as a baseline signal.

In some embodiments, analyzing the phenotype of the at least one cell is performed before segmenting a morphological image of the cell. In some embodiments, segmenting comprises detecting the cell and identifying the boundaries of the cell in the morphological image. In some embodiments, the method further comprises processing and/or transforming the morphological image to obtain images of the same cell with different readouts. In some embodiments, the images of the same cell are obtained from the at least one round of FISH, sequencing of the barcode sequence in situ, and/or segmenting of the morphological image of the cell. In some embodiments of the preceding methods, the method further comprises receiving a first image depicting the plurality of cells, wherein the first image indicates each cell of the plurality of cells by a boundary and associates each cell of the plurality of cells with a corresponding cell identifier; receiving a second image depicting locations of a plurality of barcode sequences, wherein the plurality of barcode sequences are associated with the plurality of cells after POSH is performed; aligning the first image and the second image; based on the alignment of the first image and the second image, identifying an association between each cell of the plurality of cells and a corresponding barcode sequence of the plurality of barcode sequences; receiving a FISH image of the plurality of cells after the at least one round of FISH is performed on the plurality of cells; aligning the first image and the FISH image; based on the alignment between the first image and the FISH image, resizing the FISH image; associating each cell of the plurality of cells with a portion of the resized FISH image, the corresponding barcode sequence, and the corresponding cell identifier; and analyzing known phenotypes or identifying new phenotypes of the plurality of cells. In some embodiments, the first image is generated by: receiving a third image depicting morphological characteristics of the plurality of cells; performing segmentation on the third image to generate the first image. In some embodiments, the third image is generated based on a phase image of the plurality of cells using a trained machine-learning model. In some embodiments, performing segmentation on the third image comprises computing cell and nuclei segmentation masks based on the third image. In some embodiments, the first image is obtained based on one or more FISH images of the plurality of cells. In some embodiments, the second image is generated after the FISH image. In some embodiments, the FISH image is a first FISH image corresponding to a first round of FISH, the method further comprising: receiving a second FISH image corresponding to a second round of FISH on the plurality of cells; aligning the first image and the second FISH image; based on the alignment between the first image and the second FISH image, resizing the second FISH image; and associating each cell of the plurality of biological cells with a portion of the resized first FISH image, the resized second FISH image, the corresponding barcode sequence, and the corresponding cell identifier. In some embodiments, the first FISH image and the second FISH image are captured using different microscopes. In some embodiments, the FISH image and the second image are captured using different microscopes. In some embodiments, aligning the first image and the second image comprises computing a first transformation function from the second image to the first image. In some embodiments, identifying an association between each cell of the plurality of biological cells and a corresponding barcode sequence of the plurality of barcode sequences comprises: applying the first transformation to locations of the plurality of barcode sequences in the second image to obtain corresponding locations in the second image; and comparing the corresponding locations in the second image with boundaries of the plurality of cells in the second image. In some embodiments, the first transformation is generated by: generating a reference coordinate space of the second image; extracting a patch of the second image; generating a reference coordinate space of the first image; extracting a patch of the first image; computing an affine transformation function between the patch of the second image and the patch of the first image to obtain a first plurality of transformation parameters; and generating the first transformation function based on the first plurality of transformation parameters. In some embodiments, aligning the first image and the FISH image comprises: computing a second transformation function from the first image to the FISH image. In some embodiments, resizing the FISH image comprises: obtaining one or more extremity points of a cell of the plurality of cells in the first image; applying the second transformation to locations of the one or more extremity points to obtain locations in the FISH image; based on the obtained locations in the FISH image, obtaining a boundary of the cell in the FISH image; and resizing the FISH image such that the cell in the resized FISH image is of the same or substantially similar size as the cell in the first image. In some embodiments, the second transformation is generated by: generating a reference coordinate space of the first image; extracting a patch of the first image; generating a reference coordinate space of the FISH image; extracting a patch of the FISH image; computing an affine transformation function between the patch of the first image and the patch of the FISH image to obtain a second plurality of transformation parameters; and generating the second transformation function based on the plurality of transformation parameters. In some embodiments, the patch of the first image covers a center of the first image and the patch of the FISH image covers a center of the FISH image.

In some aspects, provided herein is a method for analyzing known phenotypes or identifying new phenotypes of a plurality of cells, comprising: receiving a first image depicting the plurality of cells, wherein the first image indicates each cell of the plurality of cells by a boundary and associates each cell of the plurality of cells with a corresponding cell identifier; receiving a second image depicting locations of a plurality of barcode sequences, wherein the plurality of barcode sequences are associated with the plurality of cells after one or more in situ sequencing cycles are performed; aligning the first image and the second image; based on the alignment of the first image and the second image, identifying an association between each cell of the plurality of cells and a corresponding barcode sequence of the plurality of barcode sequences; receiving a FISH image of the plurality of cells after a FISH cycle is performed on the plurality of cells; aligning the first image and the FISH image; based on the alignment between the first image and the FISH image, resizing the FISH image; associating each cell of the plurality of cells with a portion of the resized FISH image, the corresponding barcode sequence, and the corresponding cell identifier; and analyzing known phenotypes or identifying new phenotypes of the plurality of cells. In some embodiments, the first image is generated by: receiving a third image depicting morphological characteristics of the plurality of cells; performing segmentation on the third image to generate the first image. In some embodiments, the third image is generated based on a phase image of the plurality of cells using a trained machine-learning model. In some embodiments, performing segmentation on the third image comprises computing cell and nuclei segmentation masks based on the third image. In some embodiments, the first image is obtained based on one or more FISH images of the plurality of cells. In some embodiments, the second image is generated by: amplifying the plurality of barcode sequences in the plurality of biological cells to generate barcode amplicons; iteratively sequencing the plurality of barcode amplicon sequences in situ, wherein the plurality of cells are imaged after each iteration; and generating the second image by compiling the images after each iteration of in situ sequencing. In some embodiments, the sequencing comprises sequencing by hybridization, sequencing by ligation, sequencing by synthesis, and/or sequencing by binding. In some embodiments, the second image is generated after the FISH image. In some embodiments, the FISH image is generated by: detecting DNA amplicons in the plurality of cells, wherein the detecting comprises: hybridizing an adapter oligonucleotide to the DNA amplicon; hybridizing a detection probe to the adapter oligonucleotide, wherein detection probe comprises a fluorophore, an isotope, a mass tag, an oligonucleotide, or a combination thereof; and, imaging the DNA amplicons. In some embodiments, the first image is obtained based on one or more FISH images of the plurality of cells. In some embodiments, the second image is generated after the FISH. In some embodiments, the DNA amplicons are generated by contacting a plurality of mRNA transcripts with a probe or probe set, and amplifying a plurality of target sequences using the probe or probe set as templates to form a plurality of DNA amplicons. In some embodiments, DNA amplicons are generated by: contacting a plurality of mRNA transcripts in the plurality of cells with a plurality of 3′ loop probes, wherein each 3′ loop probe comprises a first target hybridization sequence complementary to a first portion of an mRNA transcript of the plurality of mRNA transcripts; contacting the plurality of mRNA transcripts with a plurality of 5′ probes, wherein each 5′ probe comprises a second target hybridization sequence complementary to a second portion of the mRNA transcript of the plurality of mRNA transcripts, wherein each 5′ probe is capable of specifically hybridizing with a 3′ loop probe, wherein hybridization of a 5′ probe with a corresponding 3′ loop probe forms a loop in the 3′ loop probe; connecting the ends of the loop in each 3′ loop probe to form a plurality of circular probes; and amplifying a plurality of target sequences using the circular probes as templates to form a plurality of DNA amplicons. In some embodiments, the DNA amplicons are generated by: contacting a plurality of mRNA transcripts in the plurality of cells with a plurality of 5′ loop probes, wherein each 5′ loop probe comprises a first target hybridization sequence complementary to a first portion of an mRNA transcript of the plurality of mRNA transcripts; contacting the plurality of mRNA transcripts with a plurality of 3′ probes, wherein each 3′ probe comprises a second target hybridization sequence complementary to a second portion of the mRNA transcript of the plurality of mRNA transcripts, wherein each 3′ probe is capable of specifically hybridizing with a 5′ loop probe, wherein hybridization of a 3′ probe with a corresponding 5′ loop probe forms a loop in the 5′ loop probe; connecting the ends of the loop in each 5′ loop probe to form a plurality of circular probes; and amplifying a plurality of target sequences using the circular probes as templates to form a plurality of DNA amplicons. In some embodiments, the DNA amplicons are generated by RCA. In some embodiments, the FISH image is a first FISH image corresponding to a first FISH cycle, the method further comprising: receiving a second FISH image corresponding to a second FISH cycle on the plurality of cells; aligning the first image and the second FISH image; based on the alignment between the first image and the second FISH image, resizing the second FISH image; and associating each cell of the plurality of biological cells with a portion of the resized first FISH image, the resized second FISH image, the corresponding barcode sequence, and the corresponding cell identifier. In some embodiments, the method further comprises receiving a third FISH image corresponding to a third FISH cycle on the plurality of cells; aligning the third image and at least one of the first or second FISH images; based on the alignment between the third image and at least one of the first or second FISH images, resizing at least one of the first, second, or third FISH images; and associating each cell of the plurality of biological cells with a portion of the resized first, second or third FISH image, the corresponding barcode sequence, and the corresponding cell identifier. In some embodiments, the first FISH image and the second FISH image are captured using different microscopes. In some embodiments, the FISH image and the second image are captured using different microscopes. In some embodiments, aligning the first image and the second image comprises: computing a first transformation function from the second image to the first image. In some embodiments, identifying an association between each cell of the plurality of biological cells and a corresponding barcode sequence of the plurality of barcode sequences comprises: applying the first transformation to locations of the plurality of barcode sequences in the second image to obtain corresponding locations in the second image; and comparing the corresponding locations in the second image with boundaries of the plurality of cells in the second image. In some embodiments, the first transformation is generated by: generating a reference coordinate space of the second image; extracting a patch of the second image; generating a reference coordinate space of the first image; extracting a patch of the first image; computing an affine transformation function between the patch of the second image and the patch of the first image to obtain a first plurality of transformation parameters; and generating the first transformation function based on the first plurality of transformation parameters. In some embodiments, the patch of the first image covers a center of the first image and the patch of the second image covers a center of the second image. In some embodiments, aligning the first image and the FISH image comprises: computing a second transformation function from the first image to the FISH image. In some embodiments, resizing the FISH image comprises: obtaining one or more extremity points of a cell of the plurality of cells in the first image; applying the second transformation to locations of the one or more extremity points to obtain locations in the FISH image; based on the obtained locations in the FISH image, obtaining a boundary of the cell in the FISH image; and resizing the FISH image such that the cell in the resized FISH image is of the same or substantially similar size as the cell in the first image. In some embodiments, the second transformation is generated by: generating a reference coordinate space of the first image; extracting a patch of the first image; generating a reference coordinate space of the FISH image; extracting a patch of the FISH image; computing an affine transformation function between the patch of the first image and the patch of the FISH image to obtain a second plurality of transformation parameters; and generating the second transformation function based on the plurality of transformation parameters. In some embodiments, the patch of the first image covers a center of the first image and the patch of the FISH image covers a center of the FISH image.

In some aspects, provided herein is a non-transitory computer-readable storage medium storing one or more programs for analyzing known phenotypes or identifying new phenotypes of a plurality of cells, the one or more programs comprising instructions, which when executed by one or more processors of an electronic device having a display, cause the electronic device to perform the operations of: receiving a first image depicting the plurality of cells, wherein the first image indicates each cell of the plurality of cells by a boundary and associates each cell of the plurality of cells with a corresponding cell identifier; receiving a second image depicting locations of a plurality of barcode sequences, wherein the plurality of barcode sequences are associated with the plurality of cells after one or more in situ sequencing cycles are performed; aligning the first image and the second image; based on the alignment of the first image and the second image, identifying an association between each cell of the plurality of cells and a corresponding barcode sequence of the plurality of barcode sequences; receiving a FISH image of the plurality of cells after a FISH cycle is performed on the plurality of cells; aligning the first image and the FISH image; based on the alignment between the first image and the FISH image, resizing the FISH image; associating each cell of the plurality of cells with a portion of the resized FISH image, the corresponding barcode sequence, and the corresponding cell identifier; and analyzing known phenotypes or identifying new phenotypes of the plurality of cells.

In some aspects, provided herein is a system for analyzing known phenotypes or identifying new phenotypes of a plurality of cells, comprising: one or more processors; a memory; and one or more programs, wherein the one or more programs are stored in the memory and configured to be executed by the one or more processors, the one or more programs including instructions for: receiving a first image depicting the plurality of cells, wherein the first image indicates each cell of the plurality of cells by a boundary and associates each cell of the plurality of cells with a corresponding cell identifier; receiving a second image depicting locations of a plurality of barcode sequences, wherein the plurality of barcode sequences are associated with the plurality of cells after one or more in situ sequencing cycles are performed; aligning the first image and the second image; based on the alignment of the first image and the second image, identifying an association between each cell of the plurality of cells and a corresponding barcode sequence of the plurality of barcode sequences; receiving a FISH image of the plurality of cells after a FISH cycle is performed on the plurality of cells; aligning the first image and the FISH image; based on the alignment between the first image and the FISH image, resizing the FISH image; associating each cell of the plurality of cells with a portion of the resized FISH image, the corresponding barcode sequence, and the corresponding cell identifier; and analyzing known phenotypes or identifying new phenotypes of the plurality of cells.

In some embodiments of the preceding methods, the preceding non-transitory computer-readable storage medium, or the preceding system, the FISH cycle is an RNA FISH cycle, an RNA foci FISH cycle, or an immunoFISH cycle.

### BRIEF DESCRIPTION OF THE DRAWINGS

Representative embodiments of the invention are disclosed by reference to the following figures. It should be understood that the embodiments depicted are not limited to the precise details shown.

FIG. 1 illustrates an exemplary workflow of certain methods described herein, in accordance with some embodiments.

FIG. 2 illustrates an exemplary schematic for a method of introducing barcoded genetic perturbations into a plurality of cells, in accordance with some embodiments. Padlock sequences (Pad), puromycin genes (PURO), CAG promoter (CAG), and Cas9 gene (CAS9) are identified.

FIG. 3 illustrates an exemplary schematic for a method of RNA in situ fluorescence hybridization, in accordance with some embodiments.

FIG. 4 illustrates an exemplary schematic for a method of pooled optical screening of genetically barcoded cells, in accordance with some embodiments.

FIG. 5 illustrates an exemplary schematic for the methods described herein, in accordance with some embodiments.

FIG. 6 illustrates an exemplary process for analyzing known phenotypes or identifying new phenotypes of a plurality of cells, in accordance with some embodiments.

FIG. 7 illustrates an exemplary computer-implemented process for aligning between a first set of one or more images and the second set of one or more images, in accordance with some embodiments, in accordance with some embodiments.

FIGS. 8A-8F illustrate steps of an exemplary process for processing exemplary images to analyzing known phenotypes or identifying new phenotypes of two cells, in accordance with some embodiments.

FIG. 9 illustrates exemplary images of various readouts of four cells (one cell per row) obtained across four cycles of fluorescence in situ hybridization (FISH), CellPaint, and corresponding cell segmentation, in accordance with some embodiments.

FIG. 10 illustrates exemplary results of a genetic perturbation screen performed using the methods described herein, in accordance with some embodiments.

FIGS. 11A-11C illustrate the images of FIG. 9 split into three color channels for the first five columns: blue (FIG. 11A), green (FIG. 11B), and red (FIG. 11C).

FIG. 12 illustrates an exemplary workflow for an immunoFISH protocol within a FISH and pool optical screening in human cells (POSH) method provided herein, in accordance with some embodiments.

FIG. 13 illustrates an exemplary antibody-DNA conjugate design comprising primary or secondary antibodies conjugated to single-stranded DNA (ssDNA) tags, for use in, for example, an immunoFISH method provided herein, in accordance with some embodiments.

FIG. 14 illustrates an exemplary antibody-DNA conjugate design comprising primary or secondary antibodies conjugated to streptavidin and bound to biotinylated ssDNA tags, for use in, for example, an immunoFISH method provided herein, in accordance with some embodiments.

FIG. 15 illustrates an exemplary immunoFISH, POSH, and CellPaint workflow in the absence of FISH, in accordance with some embodiments.

FIG. 16 illustrates an exemplary immunoFISH, POSH, and CellPaint workflow in addition to FISH, in accordance with some embodiments.

FIG. 17 illustrates an exemplary RNA foci FISH and POSH workflow, in accordance with some embodiments.

FIG. 18 illustrates the outcome on the same population of cells using immunoFISH, RNA FISH, POSH, CellPaint, and antibody staining in a single unified workflow (i.e., immunoFISH-FISH-POSH-CP-Ab; top row), the single unified workflow without RNA FISH (middle row), or each assay performed individually (bottom row).

FIG. 19 illustrates the outcome on the same population of cells using immunoFISH, antibody staining, RNA FISH, CellPaint, and POSH in a single unified workflow (i.e., immunoFISH-Ab-FISH-CP-POSH; top row), RNA FISH-POSH and CellPaint only in a single unified workflow (middle row), or each assay performed individually (bottom row).

### DETAILED DESCRIPTION OF THE INVENTION

In one aspect, the invention provides methods for pooled optical screening of cells comprising a genetic perturbation and a barcode sequence identifying the genetic perturbation with transcriptional measurements in a combined assay. The ability to conduct in situ sequencing of barcoded genetic perturbations enables new potential applications to screen for phenotypes; however, it is also useful to measure gene expression within these same samples. In the provided methods, gene expression of cells comprising engineered genetic perturbations can be assessed using spatial transcriptomics (i.e., fluorescence in situ hybridization (FISH), such as one or more of RNA FISH (such as SNAIL FISH), or RNA foci FISH) and spatial protein identity and abundance can be assessed (such as by immunoFISH) while maintaining the molecular integrity of the cells to allow for downstream in situ sequencing (such as by POSH). The method is compatible with a variety of other protein and morphological assays as described herein. The result is a method combining transcriptional, proteomic, protein/morphological, and genetic perturbation data into a single, unified, and pooled workflow.

Utilizing the provided methods, cellular transcripts and barcode sequences can both be preserved by fixation, gene expression (mRNA) can be amplified and measured via an RNA FISH assay (such as SNAIL FISH) and/or RNA foci FISH, spatial protein identity and abundance can be measured (such as by immunoFISH), morphological phenotypes can be evaluated, and in situ sequencing can identify the barcode sequence, thus matching the genetic perturbation to the spatial transcriptomic and proteomic data and, optionally, to other phenotypic and morphological readouts. The methods allow for the detection of the presence and location of one or more proteins of interest (e.g., protein FISH, that is, FISH combining immunohistochemistry, such as immunoFISH). The methods also allow for the fixation/stabilization and detection of the presence, identity, and location of RNA foci (i.e., RNA foci FISH). In order to obtain images of the same cell with different readouts across multiple rounds of imaging (e.g., various FISH assays, morphological phenotypes, and in situ sequencing), the images can be processed such that each cell can be associated with a cell identifier, a portion of each FISH image (whether RNA FISH (including SNAIL FISH), RNA foci FISH, and/or immunoFISH), various morphological images, and the corresponding barcode sequence. Accordingly, in some embodiments, the methods comprise computational analyses to process images, including aligning images, identifying cells, and assigning genotypes, phenotypes, and morphological identifiers to individual cells.

### Definitions

As used herein, the singular forms “a.” “an,” and “the” include the plural references unless the context clearly dictates otherwise.

Reference to “about” a value or parameter herein includes (and describes) variations that are directed to that value or parameter per se. For example, description referring to “about X” includes description of “X”.

It is understood that aspects and variations of the invention described herein include “consisting” and/or “consisting essentially of” aspects and variations.

The terms “about” refers to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” should be assumed to be within an acceptable error range for that particular value or composition.

The term “optional” or “optionally” means that the subsequent described event, circumstance or substituent may or may not occur, and that the description includes instances where the event or circumstance occurs and instances where it does not.

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humor, vitreous humor, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

As used herein, “isogenic” refers to organisms or cells that are characterized by essentially identical genomic DNA, for example the genomic DNA is at least about 92%, preferably at least about 98%, and most preferably at least about 99%, identical to the genomic DNA of an isogenic organism or cell.

The term “cell” is used herein in its broadest sense in the art to mean a living body that is a structural unit of tissue of a multicellular organism, surrounded by a membrane structure separated from the outside, has genetic information and has a mechanism for expression of genetic information. The cells used herein may be naturally occurring cells or artificially modified cells (for example, fused cells, genetically modified cells, etc.).

The term “differentiated cell” as used herein can refer to a cell that has been developed from an undifferentiated phenotype to a specialized phenotype. For example, embryonic cells can differentiate into epithelial cells of the intestinal lining. Differentiated cells can be isolated from, for example, fetuses or born animals.

The term “undifferentiated cell” as used herein can refer to a progenitor cell that has an undifferentiated phenotype and is capable of differentiating. An example of an undifferentiated cell is a stem cell.

As used herein, the term “stem cell” refers to a cell capable of self-renewal and pluripotency. “Pluripotent” means that a cell can give rise to the three primary germ layers, including adult animals, through its progeny; germ cells and all three germ layers, endoderm (inner gastric lining, gastrointestinal tract, lung), mesoderm (muscle, bone, blood, genitourinary), or ectoderm (epithelial tissue and nervous system). The stem cells herein may be, but are not limited to, embryonic stem (ES) cells, tissue stem cells (also referred to as tissue-specific stem cells or somatic stem cells), or induced pluripotent stem cells. Artificially produced cells (e.g., reprogrammed cells) having the above-described capabilities may be stem cells.

The term “embryonic stem (ES) cell” as used herein can refer to a pluripotent cell isolated from an embryo maintained in an in vitro cell culture medium.

Tissue stem cells are divided into categories based on the site from which the cells are derived. For example, the skin system (e.g., epidermal stem cells, hair follicle stem cells), digestive system (e.g., pancreatic stem cells, liver stem cells, etc.), bone marrow (e.g., hematopoietic stem cells, mesenchymal stem cells, etc.) and nervous systems (e.g., neural stem cells, retinal stem cells, etc.).

“Induced pluripotent stem cells”, generally abbreviated as iPS cells or iPSCs, are pluripotent stem cells derived from non-pluripotent cells, typically adult somatic cells, or terminally differentiated cells such as fibroblasts, hematopoietic cells, muscle cells, or nerve cells. This refers to a type of pluripotent stem cell that has been artificially prepared by expressing reprogramming factors from temporarily differentiated cells such as epithelial cells.

The term “sequencing” herein refers to a method for determining the nucleotide sequence of a polynucleotide.

The term “optical phenotype” used herein refers to the use of microscopy to study a cellular phenotype.

When a range of values is provided, it is to be understood that each intervening value between the upper and lower limit of that range, and any other stated or intervening value in that stated range, is encompassed within the scope of the present disclosure. Where the stated range includes upper or lower limits, ranges excluding either of those included limits are also included in the present disclosure.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The disclosures of all publications, patents, and patent applications referred to herein are each hereby incorporated by reference in their entireties. To the extent that any reference incorporated by reference conflicts with the instant disclosure, the instant disclosure shall control.

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference. The section headings used herein are for organizational purposes only and are not to be construed as limiting the subject matter described.

### Methods of the Assay

The methods provided herein combine different techniques into a single, unified/combined workflow, allowing the measurement of transcriptional signatures (such as mRNA) and protein identity and abundance in vitro without disrupting the cells' morphology, and still enabling nucleic acids of the cells to be sequenced downstream to identify genetic perturbations, or natural (e.g., endogenous) genetic variations, associated with the observed readouts and protein/optical morphologies to be observed. In some aspects, the methods incorporate fluorescence in situ hybridization (FISH) of amplified RNA transcripts (e.g., DNA amplicons, meaning DNA polynucleotides generated from templates of mRNA transcripts, referred to herein as RNA FISH) and/or FISH of protein (such as by immunoFISH) in a manner that is compatible with pooled optical screening (POSH) techniques on the same cell, and, in some embodiments, other phenotypic readouts. In some embodiments, the methods incorporate RNA foci FISH of RNA in a manner that is compatible with pooled optical screening (POSH) techniques on the same cell, and, in some embodiments, other phenotypic readouts. Previous methods, such as STARmap (spatially-resolved transcript amplicon readout mapping), as described in Wang X., et al., 2018. Science 361(6400), and POSH, such as described in Feldman D., et al., 2019. Cell 179, 787-799, have been described separately, but have not yet been successfully combined into a single, unified/combined, and compatible workflow as described herein. The methods of FISH (e.g., RNA FISH, immunoFISH, RNA foci FISH) and POSH are described herein, in addition to other assays. Such techniques were not previously compatible within the same unified workflow on the same cell. The present invention is based, at least in part, on the inventors' discovery of means of combining FISH (e.g., RNA FISH, immunoFISH, and/or RNA foci FISH), POSH, and, in some embodiments, additional proteomic and/or morphological analyses, while preserving molecular information crucial for each aforementioned techniques. The RNA FISH described herein involves targeting mRNA transcripts and preserving/stabilizing them by fixation and reverse transcription followed by amplification, such as by rolling circle amplification. Thus, while termed herein “RNA FISH,” the molecule being assessed by FISH is understood to be a DNA amplicon, which corresponds to an mRNA transcript of interest (hence “RNA” FISH). In some embodiments of the methods described herein, the method comprises one or more rounds of SNAIL FISH. See Wang et al., Science (2018) July 27; 361 (6400). The RNA foci FISH described herein targets higher copy count RNA molecules, such as present in RNA foci, without the need for reverse transcription and amplification. The immunoFISH described herein targets one or more proteins of interest using antibodies conjugated to oligonucleotides.

In some embodiments, the method comprises a combination of FISH (e.g., RNA FISH (including SNAIL FISH), immunoFISH, and/or RNA foci FISH), POSH, and, in further embodiments, other analyses of the phenotype of the cell, such as proteomic and/or morphological analysis techniques (including, but not limited to, label-free imaging, immunohistochemistry, CellPaint, and high content quantitative phase contrast imaging). In some embodiments, FISH (e.g., RNA FISH, immunoFISH, and/or RNA foci FISH), POSH, and any combination of additional proteomic and/or morphological techniques are each performed on the same cell in a sequence and manner such that each upstream step preserves the cell and/or molecules to be assessed for downstream steps. Thus, the sequence and manner of these steps allows for downstream steps to be compatible with upstream steps. In some embodiments, FISH (e.g., RNA FISH, immunoFISH, and/or RNA foci FISH), POSH, and phenotype analyses (such as CellPaint staining) are each performed on the same cell. In some embodiments, FISH (e.g., RNA FISH, immunoFISH, and/or RNA foci FISH), POSH, and phenotype analyses (such as CellPaint staining), and cell segmentation (e.g., to identify the boundaries of each cell in a plurality of cells) are each performed on the same cell.

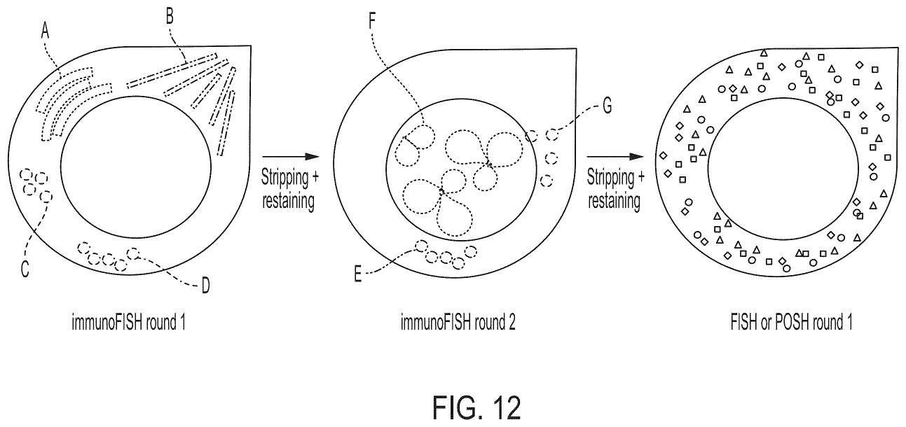

In some embodiments, the single, unified/combined workflow described herein includes one or more rounds of immunoFISH. ImmunoFISH is the combination of immunohistochemistry and FISH allowing for the detection of the presence and location of particular protein(s) of interest in the cell (or any target that can be bound by an antibody). The immunoFISH described herein is designed to be compatible with RNA FISH (e.g., FISH targeting RNA), POSH, and other assays described herein (such as CellPaint) and allows for the simultaneous staining with one or more antibodies specific for one or more target proteins of interest in one or more rounds, where each round may target a different set of biomarkers and/or proteins. As shown in FIG. 12, immunoFISH may be performed in rounds. For example, in the first round of FIG. 12, four different proteins of interest are targeted and imaged in a single cell, allowing both the presence, location, and abundance of the proteins to be assessed (such as proteins arbitrarily designated A, B. C, and D). The fluorophores responsible for the signal in the first round may then be stripped and/or otherwise neutralized, and a second round may be performed, targeting additional proteins of interest (such as proteins arbitrarily designated E, F, and G). The fluorophores from the second round may then be stripped and/or otherwise neutralized and the same cells may then undergo additional downstream analyses, including RNA FISH, POSH, and/or other assays, as otherwise described herein.

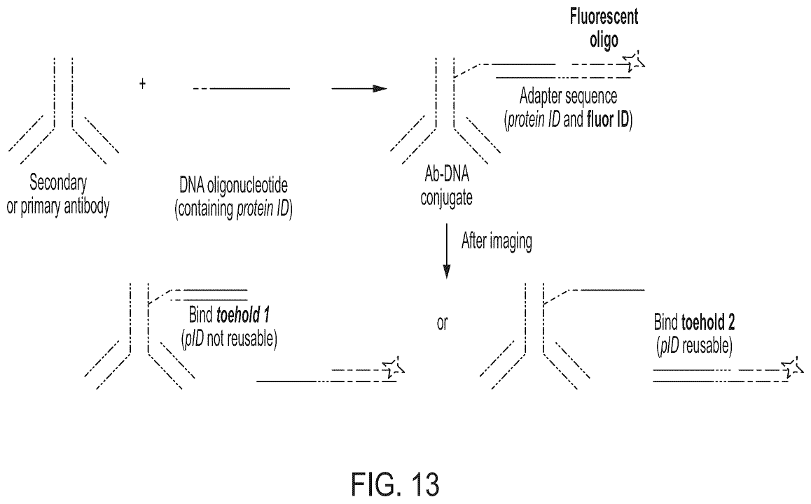

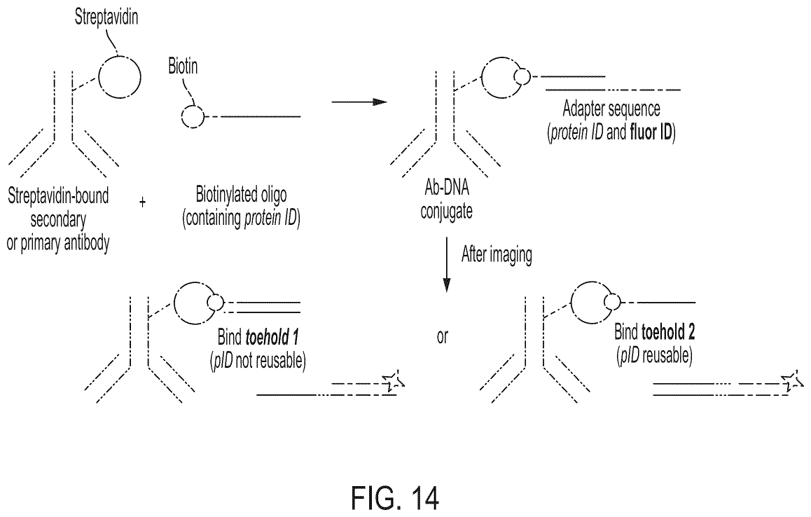

The immunoFISH described herein, in various embodiments, employs an antibody conjugate. In some embodiments, the antibody is conjugated to a DNA oligonucleotide, such as exemplified in FIG. 13 and FIG. 14. In the exemplary embodiment of FIGS. 13 and 14, the DNA oligonucleotide is single-stranded and contains a unique protein identifier/barcode uniquely associated with the protein of interest. The conjugation can be done by a variety of known methods, including, for example, by direct conjugation or by indirect linkage, such as streptavidin-biotin. The DNA oligonucleotide contains a sequence uniquely identifying the RNA biomarker, protein, or antigen (generically termed a protein ID, as shown in FIG. 13) of interest. The antibody conjugate may be a primary or a secondary antibody. If the antibody conjugate is a secondary antibody, then a primary antibody is used to first bind the RNA biomarker or protein of interest. The secondary antibody, which is conjugated to the DNA oligonucleotide, then binds the primary antibody and the DNA oligonucleotide identifies the RNA biomarker, protein, or antigen of interest that the primary antibody is bound to. If the antibody conjugate is a primary antibody, then the DNA oligonucleotide identifies the RNA biomarker or protein of interest that the antibody of the antibody conjugate is specific for. In either case, once the primary antibody conjugate or the primary antibody/secondary antibody-conjugate is bound to the RNA biomarker or protein of interest, an adapter sequence is added. The adapter sequence is complementary to a portion of the DNA oligonucleotide and further comprises an additional hybridization sequence. The additional hybridization sequence is complementary to a portion of the sequence of a fluorescent oligonucleotide. The fluorescent oligonucleotide is added and the antibody conjugate, the adapter sequence, and the fluorescent oligonucleotide form a complex (including the primary antibody if the antibody of the antibody conjugate is a secondary antibody), as shown in FIG. 13. The cells are then imaged, and the fluorescent oligonucleotide identifies the presence, abundance, and location of an RNA biomarker or protein of interest for each antibody conjugate/fluorescent oligo pair. Each immunoFISH round may target multiple targets of interest. Further, each immunoFISH round may target a control protein to control the baseline signal of the assay (for example, a regulatory protein such as GAPDH or any other suitable control protein). At the end of a round of immunoFISH, after imaging, the fluorescent oligonucleotide may be stripped, such as, in some embodiments, by use of toehold-mediated strand displacement, as shown in FIG. 13. New primary or primary and secondary antibodies may then be added for subsequent immunoFISH rounds. The fluorescent oligonucleotide from the previous rounds may be inactivated or washed away, allowing for the reuse of the same microscope channel in different immunoFISH rounds or in subsequent assay rounds (such as RNA FISH, POSH, etc.)

In some embodiments, the antibody conjugate for immunoFISH is indirectly conjugated to the DNA oligonucleotide containing the protein ID, such as by a streptavidin-biotin linker. As above, in various embodiments, the conjugate may be a primary or secondary antibody. In some embodiments, the DNA oligonucleotide containing the protein ID is biotinylated, allowing for the binding of the DNA oligonucleotide to a streptavidin-bound antibody, as shown in FIG. 14, to form the antibody conjugate. An adapter oligonucleotide and fluorescent oligonucleotide may then be added, as above, and the cells may be imaged to determine the presence, identity, and location of one or more RNA biomarkers and/or proteins of interest. After imaging, the fluorescent oligonucleotide and adapter may be removed, such as by toehold-mediated strand displacement and the fluorescent oligonucleotide may be washed away or otherwise inactivated. Subsequent rounds of immunoFISH may then be performed, targeting the same or different RNA biomarkers or proteins of interest. For example, a first round of immunoFISH may target three proteins arbitrarily designated A, B, and C and a control protein designated D (the control protein may be, for example, a regulatory protein to control the baseline signal of the assay). The second round may target three proteins arbitrarily designated E, F, and G and also the control protein designated D. The round of immunoFISH targeting A, B, and C may, in some embodiments, use the same microscope channels as the round targeting E, F, and G, since the fluorescent oligonucleotide may be washed away and/or inactivated in the first round. Likewise, downstream analyses, such as RNA FISH, POSH, or other assays may also use the same microscope channels as any round of immunoFISH. Subsequent rounds of immunoFISH may be performed. In some embodiments, at least two rounds of immunoFISH are performed, such as at least 2, at least 3, at least 4, at least 5, at least 6, or more rounds of immunoFISH. In some embodiments, each round of immunoFISH targets at least one unique protein of interest.

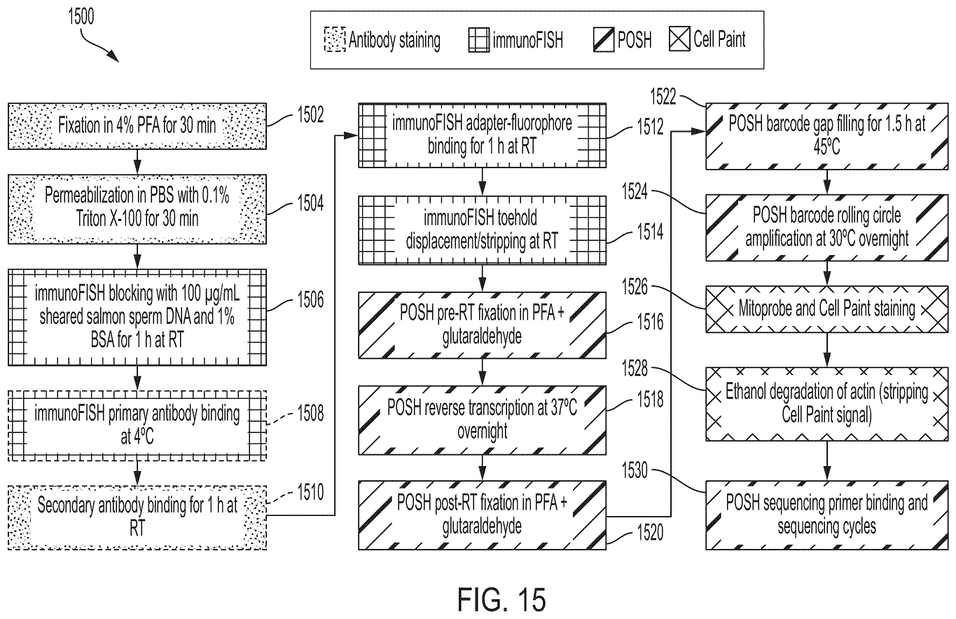

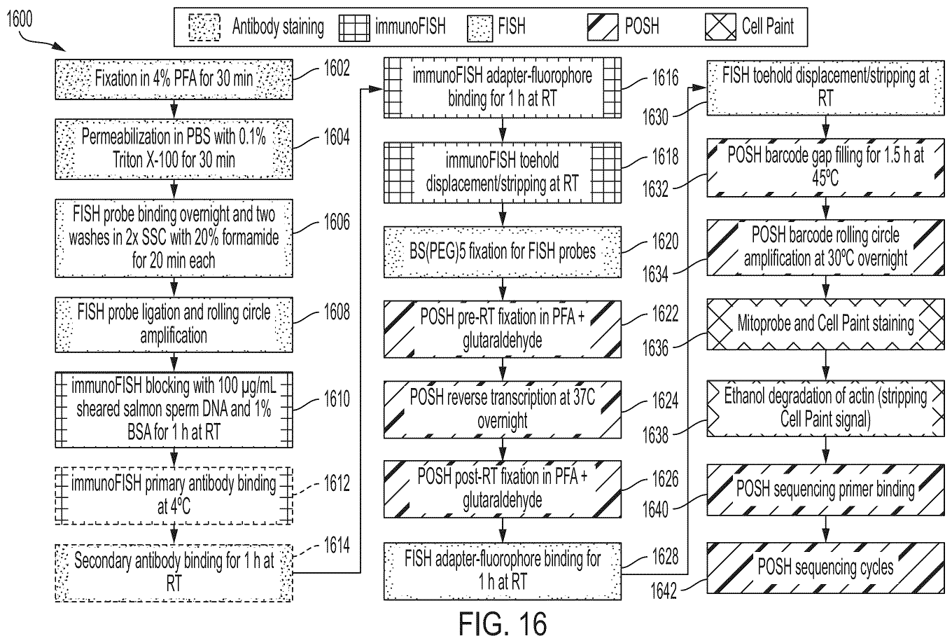

Exemplary unified workflows incorporating immunoFISH are provided in FIG. 15 and FIG. 16.

As shown in the exemplary workflow 1500 of FIG. 15, cells are fixed 1502 and permeabilized at 1504. At 1506, cells are blocked with sheared salmon sperm DNA and 1% bovine serum albumin. The immunoFISH antibody is then added to the cells and incubated at 1508 followed by secondary antibody at 1510. A combination of a primary and secondary antibody may be used. However, in some embodiments, the adapter may be bound to the primary antibody, eliminating the need for a secondary antibody. In this exemplary method, the secondary antibody is conjugated to a DNA oligonucleotide containing a sequence identifying the protein or antigen (i.e., a protein ID as shown in FIG. 13) that the primary antibody is specific for. However, in some embodiments, the primary antibody is conjugated to the DNA oligonucleotide. At 1512 immunoFISH adapters (see FIG. 13 for exemplary antibody conjugated to a DNA oligonucleotide) and a fluorescent oligonucleotide are added (see FIG. 13 for exemplary fluorescent oligo). Between 1512 and 1514, the cells may be imaged by fluorescence in one or more channels. After imaging, at 1514, toehold-mediated strand displacement (TMSD) may be used to remove the adapter sequence and fluorescent oligo from the secondary antibody (which, in some embodiments, is a primary antibody). Additional rounds of immunoFISH may be performed by returning to step 1506 or 1508. In other embodiments, imaging may occur at any time after 1512, so long as the microscope channels for the immunoFISH round are not needed for downstream assays. Once the one or more rounds of immunoFISH are completed, POSH and CellPaint protocols may then be performed at 1516, 1518, 1520, 1522, 1524, 1526, 1528, and 1530, in accordance with the protocols otherwise described herein.

The exemplary method 1500 of FIG. 15 may, in some embodiments, also include an RNA FISH method. For example, as shown in the exemplary method 1600 of FIG. 16, cells are fixed and permeabilized at 1602 and 1604, respectively. FISH probes are bound at 1606 with FISH probe ligation and rolling circle amplification performed at 1608. ImmunoFISH blocking is then performed with sheared salmon sperm DNA and BSA at 1610 followed by immunoFISH primary antibody binding at 1612. Secondary antibody is then added at 1614. In this exemplary method, the secondary antibody is conjugated to a DNA oligonucleotide containing a sequence identifying the protein or antigen (i.e., a protein ID as shown in FIG. 13) that the primary antibody specifically binds. However, in some embodiments, the primary antibody is conjugated to the DNA oligonucleotide. At 1616, immunoFISH oligonucleotide adapters (see FIG. 13 for exemplary antibody conjugated to a DNA oligonucleotide) and a fluorescent oligo are added (see FIG. 13 for exemplary fluorescent oligo). Between 1616 and 1618, the cells may be imaged for the fluorophore. After imaging, at 1618, toehold-mediated strand displacement (TMSD) may be used to remove the adapter sequence and fluorescent oligo from the second antibody (which, in some embodiments, is a primary antibody). The immunoFISH may be repeated by returning to step 1610 or 1612 to assess additional protein(s) of interest. In alternative embodiments, the cells may be imaged at any point after 1616, although preferably only if the microscope channels for the immunoFISH are available. Once the one or more rounds of immunoFISH are complete, RNA FISH, POSH, and CellPaint protocols may then be performed at 1620, 1622, 1624, 1626, 1628, 1630, 1632, 1634, 1636, 1638, 1640, and 1642, in accordance with the protocols described herein.

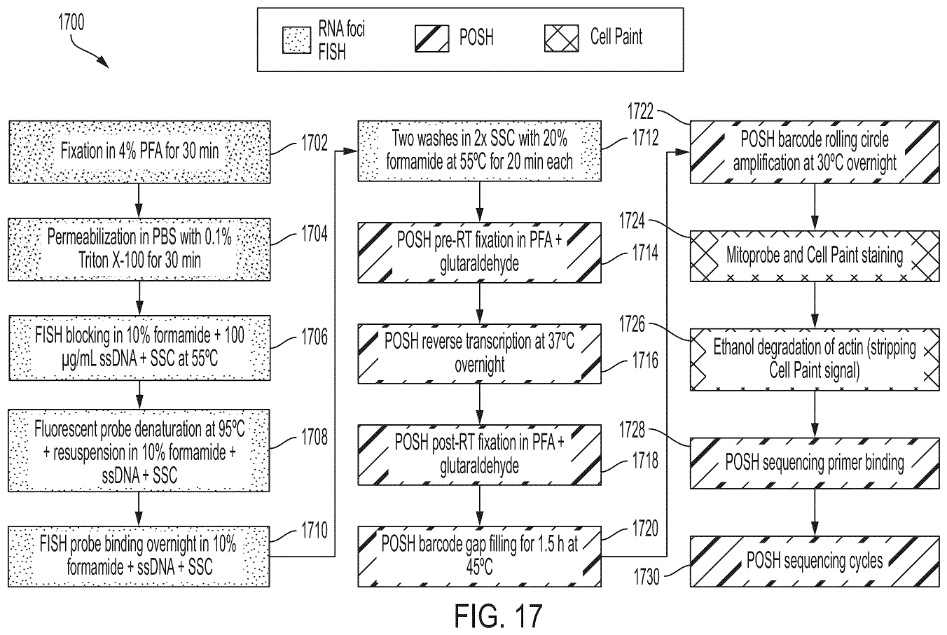

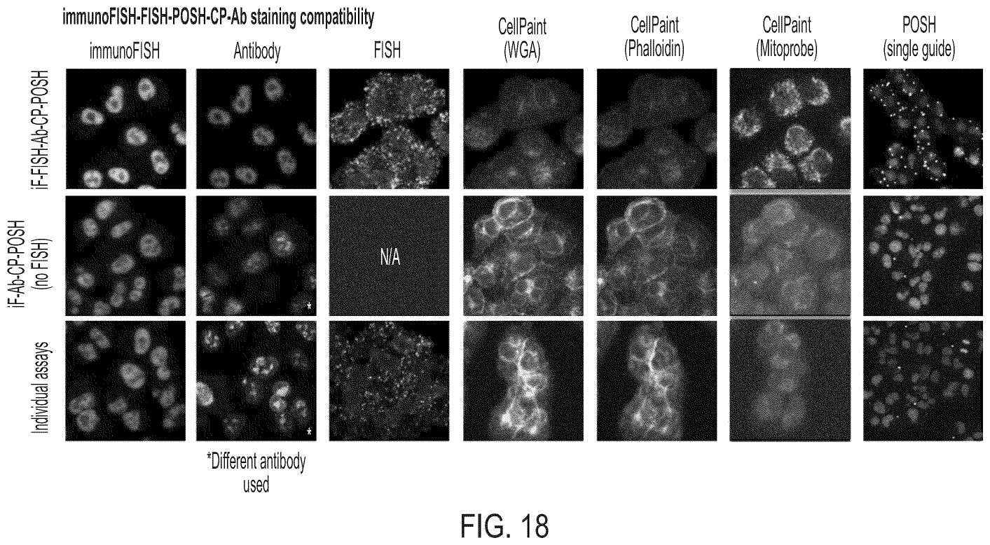

In some embodiments, the single, unified/combined workflow described herein includes one or more rounds of FISH to detect RNA, wherein the FISH is used to assess RNA without the need for reverse transcription or amplification. In some embodiments, such a method is RNA foci FISH. The use of RNA foci FISH to assess RNA in the cells, in contrast with RNA FISH otherwise described herein, allows for the detection of RNA in the cells without the need for reverse transcription or amplification by targeting RNA foci which have a higher local copy number than, for example, mRNA outside of RNA foci. In some embodiments, the probes used in RNA foci FISH are fluorescent probes. For example, in the exemplary workflow 1700 in FIG. 17, cells are fixed at 1702 and permeabilized at 1704. At 1706, the cells are blocked with 10% formamide and 100 μg/mL sheared salmon sperm DNA+SSC buffer at 55° C. Blocking at 55° C. is slightly higher than normal RNA FISH otherwise described herein, which is typically carried out below 50° C. RNA foci FISH, in some embodiments, further includes an additional washing step, but does not include the ligation and amplification steps of RNA FISH otherwise described herein. The buffer is selected to be compatible with downstream POSH. At 1708, fluorescent oligonucleotide probes are denatured at 95° C. and resuspended in 10% formamide+sheared salmon sperm DNA+SSC. These fluorescent oligonucleotides are, in some embodiments, single-stranded probes targeting repeat sequences in RNA (such as mRNA). In some embodiments, the fluorescent oligonucleotides comprise an oligonucleotide directly conjugated to a fluorophore. The FISH probes are added to the cells at 1710 and incubated overnight in 10% formamide+sheared salmon sperm DNA+SSC buffer. Cells may be imaged for FISH signal between 1712 and 1714, or at any suitable point after 1712. RNA foci FISH signal may be inactivated or removed, including, for example, to perform multiple rounds of RNA foci FISH. For example, toehold displacement could be used to remove the fluorescent oligonucleotides or RNase H/DNase digestion may be used to degrade primers. Alternatively or in addition, a different microscope channel may, in some embodiments, be used to perform multiple rounds of RNA foci FISH. The RNA foci FISH probes may then be deactivated and additional rounds of RNA foci FISH may be performed by returning to step 1706, 1708, or 1710. Once the RNA foci FISH round(s) are completed, POSH and/or additional assays may be performed (such as CellPaint) as shown in 1714, 1716, 1718, 1720, 1722, 1724, 1726, 1728, and 1730 of FIG. 17. POSH imagining may be performed in step 1730.

In some embodiments, the method comprises providing a plurality of genetically edited cells, or a plurality of cells comprising natural (e.g., endogenous) genetic variations for screening. For example, the plurality of cells may comprise at least one cell comprising at least one genetic perturbation. In some embodiments, the plurality of cells comprise a plurality of genetic perturbations, wherein a plurality of individual cells each individually comprise at least one genetic perturbation associated with a barcode sequence expressed in the cell. Each genetic perturbation can be associated with a barcode sequence, such that the barcode sequence may be used to identify the genetic perturbation associated with the cell. In some embodiments, the cells are engineered to express a Cas protein, such as Cas9. In some embodiments, the cells transiently express a Cas protein, such as Cas9. In some embodiments, the cells are engineered to constitutively express a Cas protein, such as Cas9. In some embodiments, the cells expressing the Cas protein, such as Cas9, may be genetically edited using guide RNA (gRNAs) with associated barcode sequences, where the barcode sequences may be used to identify the genetic perturbation associated with the cell. In some embodiments, the gRNA is linked to a barcode sequence. In some embodiments, the gRNA itself is a barcode sequence.

In some aspects, at least one round of FISH (such as RNA FISH, such as SNAIL FISH) is performed on a cell (i.e., a cell comprising at least one genetic perturbation associated with a barcode sequence) according to the methods described herein. Various types of FISH may be used, alone or in combination, in the unified workflow described herein, so long as they are performed in a sequence and manner that facilitates any downstream assays, such as POSH. ImmunoFISH, described herein, involves the combination of immunohistochemistry with traditional FISH wherein antibodies conjugated directly or indirectly to oligonucleotides are used to assess RNA biomarkers or proteins. RNA foci FISH, described herein, involves, in some embodiments, the use fluorescent probes to assess RNA foci. Also described herein is FISH of mRNA transcripts (variously referred to as RNA FISH or just FISH in the appropriate context). In various embodiments, RNA FISH involves amplification of the mRNA transcripts, such as by binding FISH probes and performing rolling circle amplification, followed by fixation of the amplification product, binding of adapter oligonucleotides and fluorophore oligonucleotides to the product, and then imaging of the cell by fluorescence (such as by confocal microscopy). To complete a round of RNA FISH, the fluorescent oligonucleotides may be removed, such as by toehold displacement, and new rounds of RNA FISH may be performed. Thus, in some embodiments, the at least one round of RNA FISH is associated with at least one mRNA transcript, comprising an mRNA target sequence, from a cell. Briefly, in some embodiments, a single round of RNA FISH may comprise contacting an mRNA transcript with a FISH probe set (e.g., a 3′ loop probe and a 5′ probe, or a 5′ loop probe and a 3′ probe) capable of hybridizing with a target sequence on the mRNA transcript, connecting the ends of the 3′ loop probe and the 5′ probe, or the 5′ loop probe and the 3′ probe, to form a circular probe, amplifying the target sequence (e.g., by rolling circle amplification (RCA)) to form a DNA amplicon (meaning a DNA molecule generated from an mRNA template), and detecting (e.g., by imaging) the DNA amplicon, thereby generating an RNA FISH image. In some embodiments, the 3′ loop probe and the 5′ probe are hybridized to the mRNA target sequence on the mRNA transcript prior to the connecting of the ends of the 3′ loop probe. In some embodiments, the 3′ loop probe and the 5′ probe are hybridized to the mRNA target sequence on the mRNA transcript prior to the connecting of the ends of the 3′ loop probe and prior to the amplifying of the mRNA target sequence. In some embodiments, the 3′ loop probe and the 5′ probe are hybridized to the mRNA target sequence on the mRNA transcript prior to the connecting of the ends of the 3′ loop probe, the amplifying of the mRNA target sequence, and the detection of the DNA amplicon. In some embodiments, the ends of the 3′ loop probe are connected prior to the amplifying of the mRNA target sequence. In some embodiments, the ends of the 3′ loop probe are connected prior to the amplifying of the mRNA target sequence and prior to the detection of the DNA amplicon. Alternatively, in some embodiments, the 5′ loop probe and the 3′ probe are hybridized to the mRNA target sequence on the mRNA transcript prior to the connecting of the ends of the 5′ loop probe. In some embodiments, the 5′ loop probe and the 3′ probe are hybridized to the mRNA target sequence on the mRNA transcript prior to the connecting of the ends of the 5′ loop probe and prior to the amplifying of the mRNA target sequence. In some embodiments, the 5′ loop probe and the 3′ probe are hybridized to the mRNA target sequence on the mRNA transcript prior to the connecting of the ends of the 5′ loop probe, the amplifying of the mRNA target sequence, and the detection of the DNA amplicon. In some embodiments, the ends of the 5′ loop probe are connected prior to the amplifying of the mRNA target sequence. In some embodiments, the ends of the 5′ loop probe are connected prior to the amplifying of the mRNA target sequence and prior to the detection of the DNA amplicon. In some embodiments, the mRNA target sequence is amplified prior to the detection of the DNA amplicon.

POSH serves to identify the barcode sequence associated with the genetic perturbation in a cell. In some embodiments, the cell is the same cell on which the at least one round of RNA FISH is performed. In some embodiments, the barcode sequence comprises flanking padlock probe hybridization sequences. The flanking padlock probe hybridization sequences facilitate downstream sequencing analyses. The POSH method comprises amplifying the barcode sequence in the cell and sequencing the barcode sequence in situ. More specifically, in some embodiments, POSH comprises reverse transcribing the barcode sequence, hybridizing a POSH probe (e.g., at least one padlock probe) to the reverse transcribe the barcode sequence (e.g., via padlock probe hybridization sequences flanking the reverse transcribed barcode sequence), connecting the ends of the at least one padlock probe to form a circular probe, amplifying the barcode sequence to form a barcode amplicon, and sequencing the barcode sequence in situ. In some embodiments, the sequencing in situ comprises sequencing-by-synthesis. In some embodiments, the sequencing-by-synthesis comprises generating one or more POSH images. In some embodiments, the barcode sequence is reverse transcribed prior to hybridizing the at least one padlock probe to the reverse transcribed barcode sequence. In some embodiments, the barcode sequence is reverse transcribed prior to hybridizing the at least one padlock probe to the reverse transcribed barcode sequence and prior to connecting the ends of the at least one padlock probe. In some embodiments, the barcode sequence is reverse transcribed prior to hybridizing the at least one padlock probe to the reverse transcribed barcode sequence, connecting the ends of the at least one padlock probe, and amplifying the barcode sequence. In some embodiments, the barcode sequence is reverse transcribed prior to hybridizing the at least one padlock probe to the reverse transcribed barcode sequence, connecting the ends of the at least one padlock probe, amplifying the barcode sequence, and sequencing the barcode sequence in situ. In some embodiments, the at least one padlock probe is hybridized to the reverse transcribed barcode sequence prior to connecting the ends of the at least one padlock probe. In some embodiments, the at least one padlock probe is hybridized to the reverse transcribed barcode sequence prior to connecting the ends of the at least one padlock probe and prior to amplifying the barcode sequence. In some embodiments, the at least one padlock probe is hybridized to the reverse transcribed barcode sequence prior to connecting the ends of the at least one padlock probe, amplifying the barcode sequence, and sequencing the barcode sequence in situ. In some embodiments, the ends of the at least one padlock probe are connected prior to amplifying the barcode sequence. In some embodiments, the ends of the at least one padlock probe are connected prior to amplifying the barcode sequence and prior to sequencing the barcode sequence in situ. In some embodiments, the barcode sequence is amplified prior to sequencing the barcode sequence in situ.

In some embodiments, the methods may comprise various other techniques to further analyze the phenotype of a cell. These techniques may comprises any suitable technique for analyzing the proteomic and/or morphological properties of the cell, and may include, but are not limited to, RNA foci/RNA aggregation imaging, antibody-DNA conjugate imaging, label-free imaging, high content imaging, calcium imaging, immunohistochemistry, cell morphology imaging, protein aggregation imaging, cell-cell interaction imaging, live cell imaging, and any other imaging-based assay modality (such as CellPaint). In some embodiments of the methods described herein, the cell is assessed by label-free imagining. Label-free imagining is, in some embodiments, quantitative phase contrast (QPC) imaging, brightfield imaging, autofluorescence imaging (such as under UV light), and/or fluorescent proteins engineered into the cells. The label-free imaging may be performed at any stage of the methods described herein, including before fixation (such as on live cells) or after fixation. In some embodiments, the cell is the same cell on which the at least one round of FISH is performed. In some embodiments, the cell is the same cell on which POSH is performed. In some embodiments, the cell is the same cell on which both FISH (such as RNA FISH, RNA foci FISH, immunoFISH, or a combination thereof) and POSH are performed. In some embodiments, the method comprises RNA foci/RNA aggregation imaging of the cell. In some embodiments, the method comprises antibody-DNA conjugate imaging of the cell. In some embodiments, the method comprises label-free imaging of the cell. In some embodiments, the method comprises high content imaging of the cell. In some embodiments, the method comprises calcium imaging of the cell. In some embodiments, the method comprises immunohistochemistry analysis of the cell. In some embodiments, the method comprises cell morphology imaging of the cell. In some embodiments, the method comprises protein aggregation imaging of the cell. In some embodiments, the method comprises cell-cell interaction imaging of the cell. In some embodiments, the method comprises live cell imaging of the cell. In some embodiments, the method comprises CellPaint analysis of the cell.

The methods may additionally comprise one or more processing steps performed concurrently with, prior to, or in between, the techniques described above (e.g., FISH, immunoFISH, RNA Foci FISH, POSH, and/or proteomic and/or morphological analyses). Processing steps may comprise, but are not limited to, fixation, washing (e.g., high stringency washes) and storage. The inclusion of processing steps may allow otherwise incompatible techniques, such as FISH (e.g., RNA FISH, immunoFISH, and/or RNA Foci FISH) and POSH, to be performed on the same cell without disrupting the cellular environment, that is, stabilizing or preserving the cellular environment for downstream analyses in the unified workflow. In some embodiments, the processing comprises fixation. In some embodiments, the fixation comprises paraformaldehyde (PFA), glutaraldehyde, or bis-N-succinimidyl-(pentaethylene glycol) (bis-MHS-PEG) exposure. In some embodiments, the fixation comprises PFA. In some embodiments, the fixation comprises glutaraldehyde exposure. In some embodiments, the fixation comprises PFA and glutaraldehyde exposure. In some embodiments, the fixation comprises PFA, glutaraldehyde, and bis-MHS-PEG exposure. In some embodiments, the method comprises at least one fixation via bis-MHS-PEG exposure followed by at least one fixation with PFA. In some embodiments, the processing comprises multiple rounds of fixation. In some embodiments, the process comprises sequential rounds of fixation. In some embodiments, the multiple rounds of fixation are separated by a different step in the method (e.g., FISH amplification, FISH imaging, antibody staining, proteomic/morphological analysis, POSH amplification, and/or POSH sequencing).

The methods of the present invention may comprise a particular sequence of the above described techniques, including RNA FISH, immunoFISH, RNA foci FISH, POSH, proteomic and/or morphological analysis (e.g., analyzing the phenotype), and processing. The order of these techniques, in some embodiments, allows for the preservation of molecular and morphological information without disrupting the downstream sequencing of the cell to identify perturbations. For example, in some embodiments, the techniques are integrated with one another such that certain steps of a first technique are performed before and after a second technique. In some embodiments, an RNA FISH image is generated prior to POSH image(s). In some embodiments, an RNA foci FISH image is generated prior to POSH image(s). In some embodiments, an immunoFISH image is generated prior to POSH image(s).

In some embodiments, certain steps of the FISH (such as RNA FISH) technique may be performed prior to certain steps of the POSH technique. In some embodiments, the 5′ loop probe and 3′ probe (each used in the RNA FISH technique), or the 3′ loop probe and the 5′ probe (each used in the RNA FISH technique), are hybridized to a target sequence on a mRNA transcript prior to the reverse transcription of a barcode sequence associated with a genetic perturbation in a cell. In some embodiments, the ends of the loop probe are connected, for example, the ends of the 3′ loop probe or 5′ loop probe may be ligated to one another, prior to the reverse transcription of the barcode sequence. In some embodiments, the mRNA target sequence is amplified (e.g., to generate an DNA amplicon) prior to the reverse transcription of the barcode sequence. In some embodiments, the DNA amplicon is fixed (such as by bis-MHS-PEG exposure) prior to the reverse transcription of the barcode sequence. In some embodiments, the 3′ loop probe and the 5′ probe or the 5′ loop probe and the 3′ probe are hybridized to the mRNA target sequence and the ends of the 3′ loop probe or 5′ loop probe are connected, prior to the reverse transcription of the barcode sequence. In some embodiments, the 3′ loop probe and the 5′ probe or the 5′ loop probe and the 3′ probe are hybridized to the mRNA target sequence on a mRNA transcript, the ends of the 3′ loop probe or 5′ loop probe are connected, and the mRNA target sequence is amplified, prior to the reverse transcription of the barcode sequence. In some embodiments, the 3′ loop probe and the 5′ probe, or the 5′ loop probe and the 3′ probe, are hybridized to the mRNA target sequence, the ends of the 3′ loop probe or the 5′ loop probe are connected, the mRNA target sequence is amplified, and the DNA amplicon is fixed prior to the reverse transcription of the barcode sequence.

In some embodiments, the loop probe and the probe (e.g., a 3′ loop probe and a 5′ probe, or a 5′ loop probe and a 3′ probe; each used in the RNA FISH technique) are hybridized to a target sequence on a mRNA transcript prior to the fixation of the barcode sequence (such as by PFA) associated with a genetic perturbation in a cell. The fixation of the barcode sequence may occur after the barcode sequence has been reverse transcribed. Therefore, in some embodiments, the 3′ loop probe and the 5′ probe or the 5′ loop probe and the 3′ probe are hybridized to the target mRNA sequence prior to the fixation of the reverse transcribed barcode sequence. In some embodiments, the ends of the 3′ loop probe or the 5′ loop probe are connected prior to the reverse transcription of the barcode sequence. In some embodiments, the mRNA target sequence is amplified (e.g., to generate an DNA amplicon) prior to the fixation of the reverse transcribed barcode sequence. In some embodiments, the DNA amplicon is fixed (such as by bis-MHS-PEG exposure) prior to the fixation of the reverse transcribed barcode sequence. In some embodiments, the barcode sequence is reverse transcribed prior to the fixation of the reverse transcribed barcode sequence. In some embodiments, the 3′ loop probe and the 5′ probe or the 5′ loop probe and the 3′ probe are hybridized to the mRNA target sequence and the ends of the 3′ loop probe or the 5′ loop probe are connected, prior to the fixation of the reverse transcribed barcode sequence. In some embodiments, the 3′ loop probe and the 5′ probe or the 5′ loop probe and the 3′ probe are hybridized to the mRNA target sequence, the ends of the 3′ loop probe or the 5′ loop probe are connected, and the mRNA target sequence is amplified, prior to the fixation of the reverse transcribed barcode sequence. In some embodiments, the 3′ loop probe and the 5′ probe or the 5′ loop probe and the 3′ probe are hybridized to the mRNA target sequence, the ends of the 3′ loop probe or the 5′ loop probe are connected, the mRNA target sequence is amplified, and the DNA amplicon is fixed prior to the fixation of the reverse transcribed barcode sequence. In some embodiments, the 3′ loop probe and the 5′ probe or the 5′ loop probe and the 3′ probe are hybridized to the mRNA target sequence, the ends of the 3′ loop probe or the 5′ loop probe are connected, the mRNA target sequence is amplified, the DNA amplicon is fixed, and the barcode sequence is reverse transcribed prior to the fixation of the reverse transcribed barcode sequence.

In some embodiments, the loop probe and the probe (e.g., a 3′ loop probe and a 5′ probe, or a 5′ loop probe and a 3′ probe; each used in the RNA FISH technique) are hybridized to a target sequence on a mRNA transcript prior to the detection of a DNA amplicon generated from the mRNA transcript, such as via imaging of the DNA amplicon (RNA FISH imaging). Therefore, in some embodiments, the 3′ loop probe and the 5′ probe or the 5′ loop probe and the 3′ probe are hybridized to a target sequence on a mRNA transcript prior to RNA FISH imaging. In some embodiments, the ends of the 3′ loop probe or the 5′ loop probe are connected prior to RNA FISH imaging. In some embodiments, the mRNA target sequence is amplified (e.g., to generate an DNA amplicon) prior to RNA FISH imaging. In some embodiments, the DNA amplicon is fixed (e.g., via bis-MHS-PEG exposure) prior to RNA FISH imaging. In some embodiments, the barcode sequence is reverse transcribed prior to RNA FISH imaging. In some embodiments, the reverse transcribed barcode sequence is fixed (e.g., via PFA) prior to RNA FISH imaging. In some embodiments, the 3′ loop probe and the 5′ probe or the 5′ loop probe and the 3′ probe are hybridized to the mRNA target sequence and the ends of the 3′ loop probe or the 5′ loop probe are connected, prior to RNA FISH imaging. In some embodiments, the 3′ loop probe and the 5′ probe or the 5′ loop probe and the 3′ probe are hybridized to the mRNA target sequence, the ends of the 3′ loop probe are connected, and the mRNA target sequence is amplified, prior to RNA FISH imaging. In some embodiments, the 3′ loop probe and the 5′ probe or the 5′ loop probe and the 3′ probe are hybridized to the mRNA target sequence, the ends of the 3′ loop probe or the 5′ loop probe are connected, the mRNA target sequence is amplified, and the DNA amplicon is fixed prior to RNA FISH imaging. In some embodiments, the 3′ loop probe and the 5′ probe or the 5′ loop probe and the 3′ probe are hybridized to the mRNA target sequence, the ends of the 3′ loop probe or the 5′ loop probe are connected, the mRNA target sequence is amplified, the DNA amplicon is fixed, and the barcode sequence is reverse transcribed, prior to RNA FISH imaging. In some embodiments, the 3′ loop probe and the 5′ probe or the 5′ loop probe and the 3′ probe are hybridized to the mRNA target sequence, the ends of the 3′ loop probe or the 5′ loop probe are connected, the mRNA target sequence is amplified, the DNA amplicon is fixed, the barcode sequence is reverse transcribed, and the reverse transcribed barcode sequence is fixed, prior to RNA FISH imaging.

In some embodiments, the loop probe and the probe (e.g., a 3′ loop probe and a 5′ probe, or a 5′ loop probe and a 3′ probe; each used in the RNA FISH technique) are hybridized to a target sequence on a mRNA transcript prior to the hybridization of the at least one padlock probe (used in the POSH technique) to the padlock probe hybridization sequences flanking the barcode sequence, connection of the ends of the at least one padlock probe (such as by gap filling), and amplification of the barcode sequence (e.g., via RCA, thereby generating barcode amplicons). In some embodiments, the ends of the 3′ loop probe or the 5′ loop probe are connected prior to the hybridization of the at least one padlock probe, connection of the ends of the at least one padlock probe, and amplification of the barcode sequence. In some embodiments, the mRNA target sequence is amplified (e.g., to generate an DNA amplicon) prior to the hybridization of the at least one padlock probe, connection of the ends of the at least one padlock probe, and amplification of the barcode sequence. In some embodiments, the DNA amplicon is fixed (e.g., via bis-MHS-PEG exposure) prior to the hybridization of the at least one padlock probe, connection of the ends of the at least one padlock probe, and amplification of the barcode sequence. In some embodiments, the barcode sequence is reverse transcribed prior to the hybridization of the at least one padlock probe, connection of the ends of the at least one padlock probe, and amplification of the barcode sequence. In some embodiments, the reverse transcribed barcode sequence is fixed (e.g., via PFA) prior to the hybridization of the at least one padlock probe, connection of the ends of the at least one padlock probe, and amplification of the barcode sequence. In some embodiments, the DNA amplicon is detected (e.g., the RNA FISH image is generated) prior to the hybridization of the at least one padlock probe, connection of the ends of the at least one padlock probe, and amplification of the barcode sequence. In some embodiments, the 3′ loop probe and the 5′ probe or the 5′ loop probe and the 3′ probe are hybridized to the mRNA target sequence and the ends of the 3′ loop probe or the 5′ loop probe are connected, prior to the hybridization of the at least one padlock probe, connection of the ends of the at least one padlock probe, and amplification of the barcode sequence. In some embodiments, the 3′ loop probe and the 5′ probe or the 5′ loop probe and the 3′ probe are hybridized to the mRNA target sequence, the ends of the 3′ loop probe or the 5′ loop probe are connected, and the mRNA target sequence is amplified, prior to the hybridization of the at least one padlock probe, connection of the ends of the at least one padlock probe, and amplification of the barcode sequence. In some embodiments, the 3′ loop probe and the 5′ probe or the 5′ loop probe and the 3′ probe are hybridized to the mRNA target sequence, the ends of the 3′ loop probe or the 5′ loop probe are connected, the mRNA target sequence is amplified, and the DNA amplicon is fixed prior to the hybridization of the at least one padlock probe, connection of the ends of the at least one padlock probe, and amplification of the barcode sequence. In some embodiments, the 3′ loop probe and the 5′ probe or the 5′ loop probe and the 3′ probe are hybridized to the mRNA target sequence, the ends of the 3′ loop probe or the 5′ loop probe are connected, the mRNA target sequence is amplified, the DNA amplicon is fixed, and the barcode sequence is reverse transcribed, prior to the hybridization of the at least one padlock probe, connection of the ends of the at least one padlock probe, and amplification of the barcode sequence. In some embodiments, the 3′ loop probe and the 5′ probe or the 5′ loop probe and the 3′ probe are hybridized to the mRNA target sequence, the ends of the 3′ loop probe or the 5′ loop probe are connected, the mRNA target sequence is amplified, the DNA amplicon is fixed, the barcode sequence is reverse transcribed, and the reverse transcribed barcode sequence is fixed, prior to the hybridization of the at least one padlock probe, connection of the ends of the at least one padlock probe, and amplification of the barcode sequence. In some embodiments, the 3′ loop probe and the 5′ probe or the 5′ loop probe and the 3′ probe are hybridized to the mRNA target sequence, the ends of the 3′ loop probe or the 5′ loop probe are connected, the mRNA target sequence is amplified, the DNA amplicon is fixed, the barcode sequence is reverse transcribed, the reverse transcribed barcode sequence is fixed, and the RNA FISH image is generated, prior to the hybridization of the at least one padlock probe, connection of the ends of the at least one padlock probe, and amplification of the barcode sequence.

In some embodiments, the 3′ loop probe and the 5′ probe or the 5′ loop probe and the 3′ probe are hybridized to a target sequence on a mRNA transcript prior to analyzing the phenotype of the cell, such as by RNA foci/RNA aggregation imaging, antibody-DNA conjugate imaging, label-free imaging, high content imaging, calcium imaging, immunohistochemistry, cell morphology imaging, protein aggregation imaging, cell-cell interaction imaging, live cell imaging, or any other imaging-based assay modality (e.g., CellPaint). In some embodiments, the ends of the 3′ loop probe or the 5′ loop probe are connected prior to analyzing the phenotype. In some embodiments, the mRNA target sequence is amplified (e.g., to generate an DNA amplicon) prior to analyzing the phenotype. In some embodiments, the DNA amplicon is fixed (e.g., via bis-MHS-PEG exposure) prior to analyzing the phenotype. In some embodiments, the barcode sequence is reverse transcribed prior to analyzing the phenotype. In some embodiments, the reverse transcribed barcode sequence is fixed (e.g., via PFA) prior to analyzing the phenotype. In some embodiments, the DNA amplicon is detected (e.g., the RNA FISH image is generated) prior to analyzing the phenotype. In some embodiments, the at least one padlock probe is hybridized to the padlock probe hybridization sequences flanking the barcode sequence, the ends of the at least one padlock probe are connected (e.g., via gap filling), and barcode sequence is amplified (e.g., via RCA, thereby generating barcode amplicons prior to analyzing the phenotype. In some embodiments, the 3′ loop probe and the 5′ probe or the 5′ loop probe and the 3′ probe are hybridized to the mRNA target sequence and the ends of the 3′ loop probe or the 5′ loop probe are connected, prior to analyzing the phenotype. In some embodiments, the 3′ loop probe and the 5′ probe or the 5′ loop probe and the 3′ probe are hybridized to the mRNA target sequence, the ends of the 3′ loop probe or the 5′ loop probe are connected, and the mRNA target sequence is amplified, prior to analyzing the phenotype. In some embodiments, the 3′ loop probe and the 5′ probe or the 5′ loop probe and the 3′ probe are hybridized to the mRNA target sequence, the ends of the 3′ loop probe are connected, the mRNA target sequence is amplified, and the DNA amplicon is fixed, prior to analyzing the phenotype. In some embodiments, the 3′ loop probe and the 5′ probe or the 5′ loop probe and the 3′ probe are hybridized to the mRNA target sequence, the ends of the 3′ loop probe or the 5′ loop probe are connected, the mRNA target sequence is amplified, the DNA amplicon is fixed, and the barcode sequence is reversed transcribed, prior to analyzing the phenotype. In some embodiments, the 3′ loop probe and the 5′ probe or the 5′ loop probe and the 3′ probe are hybridized to the mRNA target sequence, the ends of the 3′ loop probe or the 5′ loop probe are connected, the mRNA target sequence is amplified, the DNA amplicon is fixed, the barcode sequence is reversed transcribed, prior to analyzing the phenotype. In some embodiments, the 3′ loop probe and the 5′ probe or the 5′ loop probe and the 3′ probe are hybridized to the mRNA target sequence, the ends of the 3′ loop probe are connected, the mRNA target sequence is amplified, the DNA amplicon is fixed, the barcode sequence is reversed transcribed, and the reverse transcribed barcode sequence is fixed, prior to analyzing the phenotype. In some embodiments, the 3′ loop probe and the 5′ probe or the 5′ loop probe and the 3′ probe are hybridized to the mRNA target sequence, the ends of the 3′ loop probe or the 5′ loop probe are connected, the mRNA target sequence is amplified, the DNA amplicon is fixed, the barcode sequence is reversed transcribed, the reverse transcribed barcode sequence is fixed, and the RNA FISH image is generated, prior to analyzing the phenotype. In some embodiments, the 3′ loop probe and the 5′ probe or the 5′ loop probe and the 3′ probe are hybridized to the mRNA target sequence, the ends of the 3′ loop probe or the 5′ loop probe are connected, the mRNA target sequence is amplified, the DNA amplicon is fixed, the barcode sequence is reversed transcribed, the reverse transcribed barcode sequence is fixed, the RNA FISH image is generated, the at least one padlock probe is hybridized to the padlock probe hybridization sequences flanking the barcode sequence, the ends of the at least one padlock probe are connected, and the barcode sequence is amplified, prior to analyzing the phenotype.

In some embodiments, the 3′ loop probe and the 5′ probe or the 5′ loop probe and the 3′ probe are hybridized to a target sequence on a mRNA transcript prior to sequencing a barcode sequence, associated with a genetic perturbation in a cell, in situ. The in situ sequencing may comprise sequencing-by-synthesis, thereby generating a series of images corresponding to the sequence of the barcode (e.g., POSH image(s)). In some embodiments, the ends of the 3′ loop probe are connected prior to sequencing the barcode sequence in situ. In some embodiments, the mRNA target sequence is amplified (e.g., to generate an DNA amplicon) prior sequencing the barcode sequence in situ. In some embodiments, the DNA amplicon is fixed (such as by bis-MHS-PEG exposure) prior to sequencing the barcode sequence in situ. In some embodiments, the barcode sequence is reverse transcribed prior to sequencing the barcode sequence in situ. In some embodiments, the reverse transcribed barcode sequence is fixed (such as by PFA) prior to sequencing the barcode sequence in situ. In some embodiments, the DNA amplicon is detected (i.e., the RNA FISH image is generated) prior to sequencing the barcode sequence in situ. In some embodiments, the at least one padlock probe is hybridized to the padlock probe hybridization sequences flanking the barcode sequence, the ends of the at least one padlock probe are connected (such as by gap filling), and barcode sequence is amplified (such as by RCA), thereby generating barcode amplicons, prior to sequencing the barcode sequence in situ. In some embodiments, the phenotype of the cell is analyzed, such as by RNA foci/RNA aggregation imaging, antibody-DNA conjugate imaging, label-free imaging, high content imaging, calcium imaging, immunohistochemistry, cell morphology imaging, protein aggregation imaging, cell-cell interaction imaging, live cell imaging, or any other imaging-based assay modality (e.g., CellPaint), prior to sequencing the barcode sequence in situ. In some embodiments, the 3′ loop probe and the 5′ probe or the 5′ loop probe and the 3′ probe are hybridized to the mRNA target sequence and the ends of the 3′ loop probe or the 5′ loop probe are connected, sequencing the barcode sequence in situ. In some embodiments, the 3′ loop probe and the 5′ probe or the 5′ loop probe and the 3′ probe are hybridized to the mRNA target sequence, the ends of the 3′ loop probe are connected, and the mRNA target sequence is amplified, prior to sequencing the barcode sequence in situ. In some embodiments, the 3′ loop probe and the 5′ probe or the 5′ loop probe and the 3′ probe are hybridized to the mRNA target sequence, the ends of the 3′ loop probe or the 5′ loop probe are connected, the mRNA target sequence is amplified, and the DNA amplicon is fixed, prior to sequencing the barcode sequence in situ. In some embodiments, the 3′ loop probe and the 5′ probe or the 5′ loop probe and the 3′ probe are hybridized to the mRNA target sequence, the ends of the 3′ loop probe or the 5′ loop probe are connected, the mRNA target sequence is amplified, the DNA amplicon is fixed, and the barcode sequence is reversed transcribed, prior to sequencing the barcode sequence in situ. In some embodiments, the 3′ loop probe and the 5′ probe or the 5′ loop probe and the 3′ probe are hybridized to the mRNA target sequence, the ends of the 3′ loop probe are connected, the mRNA target sequence is amplified, the DNA amplicon is fixed, the barcode sequence is reversed transcribed, and the reverse transcribed barcode sequence is fixed, prior to sequencing the barcode sequence in situ. In some embodiments, the 3′ loop probe and the 5′ probe or the 5′ loop probe and the 3′ probe are hybridized to the mRNA target sequence, the ends of the 3′ loop probe or the 5′ loop probe are connected, the mRNA target sequence is amplified, the DNA amplicon is fixed, the barcode sequence is reversed transcribed, the reverse transcribed barcode sequence is fixed, and the RNA FISH image is generated, prior to sequencing the barcode sequence in situ. In some embodiments, the 3′ loop probe and the 5′ probe or the 5′ loop probe and the 3′ probe are hybridized to the mRNA target sequence, the ends of the 3′ loop probe or the 5′ loop probe are connected, the mRNA target sequence is amplified, the DNA amplicon is fixed, the barcode sequence is reversed transcribed, the reverse transcribed barcode sequence is fixed, the RNA FISH image is generated, and the at least one padlock probe is hybridized to the padlock probe hybridization sequences flanking the barcode sequence, the ends of the at least one padlock probe are connected, and barcode sequence is amplified, prior to sequencing the barcode sequence in situ. In some embodiments, the 3′ loop probe and the 5′ probe or the 5′ loop probe and the 3′ probe are hybridized to the mRNA target sequence, the ends of the 3′ loop probe or the 5′ loop probe are connected, the mRNA target sequence is amplified, the DNA amplicon is fixed, the barcode sequence is reversed transcribed, the reverse transcribed barcode sequence is fixed, the RNA FISH image is generated, the at least one padlock probe is hybridized to the padlock probe hybridization sequences flanking the barcode sequence, the ends of the at least one padlock probe are connected, the barcode sequence is amplified, and the phenotype of the cell is analyzed, prior to sequencing the barcode sequence in situ.

In some embodiments, the unified workflow method comprises the following steps:
(1) hybridizing RNA FISH probes to a target sequence of an mRNA transcript in a cell comprising a genetic perturbation and a barcode identifying the genetic perturbation;
(2) connecting the ends of the RNA FISH probes (such as by ligation) to form a circular probe;
(3) amplifying the mRNA target sequence (such as by RCA), thereby generating an DNA amplicon;
(4) fixing the DNA amplicon in place, such as by bis-MHS-PEG exposure;
(5) reverse transcribing a barcode sequence that corresponds to a genetic perturbation in the cell;
(6) fixing the reverse transcribed barcode sequence, optionally via a PFA, glutaraldehyde, and/or PEG treatment;
(7) detecting (e.g., imaging) the DNA amplicon, thereby generating an RNA FISH image;
(8) hybridizing at least one padlock probe to padlock probe hybridization sequences flanking the reverse transcribed barcode sequence, connecting the ends of the at least one padlock probe (e.g., via gap filling), and amplifying the reverse transcribed barcode sequence, thereby generating barcode sequence amplicons; and,
(9) sequencing the barcode sequence in situ (e.g., via sequencing-by-synthesis), thereby generating POSH image(s).

In some embodiments, the method is performed in numerical order of steps 1-9. In some embodiments, the method is performed out of numerical order of steps 1-9. In some embodiments, the method comprises steps in addition to steps 1-9. In some embodiments, the method comprises omitting one or more of steps 1-9.

In some embodiments, the method comprises the following steps:
(1) hybridizing a 3′ loop probe and a 5′ probe or a 5′ loop probe and a 3′ probe (i.e., RNA FISH probes; such as SNAIL probes) to a target sequence of an mRNA transcript in a cell;
(2) connecting the ends of the 3′ loop probe or the 5′ loop probe (such as by ligation) to form a circular probe;
(3) amplifying the mRNA target sequence (such as by RCA), thereby generating an DNA amplicon;
(4) fixing the DNA amplicon in place, such as by bis-MHS-PEG exposure;
(5) reverse transcribing a barcode sequence that corresponds to a genetic perturbation in the cell;
(6) fixing the reverse transcribed barcode sequence, optionally via a PFA, glutaraldehyde, and/or PEG treatment;
(7) detecting (e.g., imaging) the DNA amplicon, thereby generating an RNA FISH image;
(8) hybridizing at least one padlock probe to padlock probe hybridization sequences flanking the reverse transcribed barcode sequence, connecting the ends of the at least one padlock probe (e.g., via gap filling), and amplifying the reverse transcribed barcode sequence, thereby generating barcode sequence amplicons; and,
(9) sequencing the barcode sequence in situ (e.g., via sequencing-by-synthesis), thereby generating POSH image(s).

In some embodiments, the method is performed in numerical order of steps 1-9. In some embodiments, the method is performed out of numerical order of steps 1-9. In some embodiments, the method comprises steps in addition to steps 1-9. In some embodiments, the method comprises omitting one or more of steps 1-9.

In some embodiments, the method comprises the following steps:
(1) hybridizing a 3′ loop probe and a 5′ probe or a 5′ loop probe and a 3′ probe (i.e., RNA FISH probes; such as SNAIL probes) to a target sequence of an mRNA transcript in a cell;
(2) connecting the ends of the 3′ loop probe or the 5′ loop probe (such as by ligation) to form a circular probe;
(3) amplifying the mRNA target sequence (such as by RCA), thereby generating an DNA amplicon;
(4) fixing the DNA amplicon in place, such as by bis-MHS-PEG exposure;
(5) reverse transcribing a barcode sequence that corresponds to a genetic perturbation in the cell;
(6) fixing the reverse transcribed barcode sequence, optionally via a PFA, glutaraldehyde, and/or PEG treatment;
(7) detecting (e.g., imaging) the DNA amplicon, thereby generating an RNA FISH image;
(8) hybridizing at least one padlock probe to padlock probe hybridization sequences flanking the reverse transcribed barcode sequence, connecting the ends of the at least one padlock probe (e.g., via gap filling), and amplifying the reverse transcribed barcode sequence, thereby generating barcode sequence amplicons;
(9) analyzing the phenotype of the cell (e.g., RNA foci/RNA aggregation imaging, antibody-DNA conjugate imaging, label-free imaging, high content imaging, calcium imaging, immunohistochemistry, cell morphology imaging, protein aggregation imaging, cell-cell interaction imaging, live cell imaging, or any other imaging-based assay modality (e.g., CellPaint)); and,
(10) sequencing the barcode sequence in situ (e.g., via sequencing-by-synthesis), thereby generating POSH image(s).

In some embodiments, the method is performed in numerical order of steps 1-10. In some embodiments, the method is performed out of numerical order of steps 1-10. In some embodiments, the method comprises steps in addition to steps 1-10. In some embodiments, the method comprises omitting one or more of steps 1-10.

In some embodiments, the method comprises the following steps:
(1) hybridizing a 3′ loop probe and a 5′ probe or a 5′ loop probe and a 3′ probe (i.e., RNA FISH probes; such as SNAIL probes) to a target sequence of an mRNA transcript in a cell;
(2) connecting the ends of the 3′ loop probe or the 5′ loop probe (such as by ligation) to form a circular probe;
(3) amplifying the mRNA target sequence (such as by RCA), thereby generating an DNA amplicon;
(4) fixing the DNA amplicon in place, such as by bis-MHS-PEG exposure;
(5) reverse transcribing a barcode sequence that corresponds to a genetic perturbation in the cell;
(6) fixing the reverse transcribed barcode sequence, optionally via a PFA, glutaraldehyde, and/or PEG treatment;
(7) detecting (e.g., imaging) the DNA amplicon, thereby generating a n RNAFISH image;
(8) hybridizing at least one padlock probe to padlock probe hybridization sequences flanking the reverse transcribed barcode sequence, connecting the ends of the at least one padlock probe (e.g., via gap filling), and amplifying the reverse transcribed barcode sequence, thereby generating barcode sequence amplicons;
(9) analyzing the phenotype of the cell (e.g., RNA foci/RNA aggregation imaging, antibody-DNA conjugate imaging, label-free imaging, high content imaging, calcium imaging, immunohistochemistry, cell morphology imaging, protein aggregation imaging, cell-cell interaction imaging, live cell imaging, or any other imaging-based assay modality (e.g., CellPaint));
(10) sequencing the barcode sequence in situ (e.g., via sequencing-by-synthesis), thereby generating POSH image(s); and,
(11) segmenting a morphological image of the cell to detect the plurality of cells and identify the boundaries of the plurality of cells in the morphological image.

In some embodiments, the method is performed in numerical order of steps 1-11. In some embodiments, the method is performed out of numerical order of steps 1-11. In some embodiments, the method comprises steps in addition to steps 1-11. In some embodiments, the method comprises omitting one or more of steps 1-11.

In some embodiments, the method comprises the following steps:
(1) hybridizing a 3′ loop probe and a 5′ probe or a 5′ loop probe and a 3′ probe (i.e., RNA FISH probes; such as SNAIL probes) to a target sequence of an mRNA transcript in a cell;
(2) connecting the ends of the 3′ loop probe or the 5′ loop probe (such as by ligation) to form a circular probe;
(3) amplifying the mRNA target sequence (such as by RCA), thereby generating an DNA amplicon;
(4) fixing the DNA amplicon in place, such as by bis-MHS-PEG exposure;
(5) reverse transcribing a barcode sequence that corresponds to a genetic perturbation in the cell;
(6) fixing the reverse transcribed barcode sequence, optionally via a PFA and/or glutaraldehyde treatment;
(7) detecting (e.g., imaging) the DNA amplicon, thereby generating an RNA FISH image;
(8) hybridizing at least one padlock probe to padlock probe hybridization sequences flanking the reverse transcribed barcode sequence, connecting the ends of the at least one padlock probe (e.g., via gap filling), and amplifying the reverse transcribed barcode sequence, thereby generating barcode sequence amplicons;
(9) analyzing the phenotype of the cell (e.g., RNA foci/RNA aggregation imaging, antibody-DNA conjugate imaging, label-free imaging, high content imaging, calcium imaging, immunohistochemistry, cell morphology imaging, protein aggregation imaging, cell-cell interaction imaging, live cell imaging, or any other imaging-based assay modality (e.g., CellPaint));
(10) sequencing the barcode sequence in situ (e.g., via sequencing-by-synthesis), thereby generating POSH image(s);
(11) segmenting a morphological image of the cell to detect the plurality of cells and identify the boundaries of the plurality of cells in the morphological image; and,
(12) processing and/or transforming the images (e.g., the RNA FISH image(s), the POSH image(s), and/or the segmentation image(s)) using computational analysis to obtain images of the same cell with different readouts.

In some embodiments, the method is performed in numerical order of steps 1-12. In some embodiments, the method is performed out of numerical order of steps 1-12. In some embodiments, the method comprises steps in addition to steps 1-12. In some embodiments, the method comprises omitting one or more of steps 1-12.

In some embodiments, the method comprises the following steps:
(1) contacting at least one cell with a conjugate comprising an antibody that specifically binds at least one protein of interest (i.e., an immunoFISH primary antibody) and a DNA oligonucleotide comprising a protein identification sequence uniquely associated with the protein of interest;
(2) contacting the conjugate with an adapter oligonucleotide (i.e., an immunoFISH adapter oligonucleotide), wherein the adapter oligonucleotide comprises a first adapter sequence complementary to a portion of the DNA oligonucleotide;
(3) contacting the adapter oligonucleotide with a fluorescent oligonucleotide (i.e., an immunoFISH fluorescent oligonucleotide) capable of hybridizing with a second adapter sequence of the adapter oligonucleotide;
(4) imaging the at least one cell to detect the at least one fluorescent oligonucleotide, thereby generating immunoFISH image(s);
(5) reverse transcribing a barcode sequence that corresponds to a genetic perturbation in the cell;
(6) fixing the reverse transcribed barcode sequence, optionally via a PFA and/or glutaraldehyde treatment;
(7) hybridizing at least one padlock probe to padlock probe hybridization sequences flanking the reverse transcribed barcode sequence, connecting the ends of the at least one padlock probe (e.g., via gap filling), and amplifying the reverse transcribed barcode sequence, thereby generating barcode sequence amplicons;
(8) analyzing the phenotype of the cell (e.g., RNA foci/RNA aggregation imaging, antibody-DNA conjugate imaging, label-free imaging, high content imaging, calcium imaging, immunohistochemistry, cell morphology imaging, protein aggregation imaging, cell-cell interaction imaging, live cell imaging, or any other imaging-based assay modality (e.g., CellPaint));
(9) sequencing the barcode sequence in situ (e.g., via sequencing-by-synthesis), thereby generating POSH image(s);
(10) segmenting a morphological image of the cell to detect the plurality of cells and identify the boundaries of the plurality of cells in the morphological image; and,
(11) processing and/or transforming the images (e.g., the immunoFISH image(s), the POSH image(s), and/or the segmentation image(s)) using computational analysis to obtain images of the same cell with different readouts.

In some embodiments, the method is performed in numerical order of steps 1-11. In some embodiments, the method is performed out of numerical order of steps 1-11. In some embodiments, the method comprises steps in addition to steps 1-11. In some embodiments, the method comprises omitting one or more of steps 1-11.

In some embodiments, the method comprises the following steps:
(1) contacting at least one cell with a first antibody which specifically binds at least one protein of interest (i.e., an immunoFISH primary antibody) and a DNA oligonucleotide comprising a protein identification sequence uniquely associated with the protein of interest;
(2) contacting the antibody with a conjugate comprising an antibody which specifically binds the first antibody and a DNA oligonucleotide comprising a protein identification sequence uniquely associated with the protein of interest;
(3) contacting the conjugate with an adapter oligonucleotide (i.e., an immunoFISH primary antibody), wherein the adapter oligonucleotide comprises a first adapter sequence complementary to a portion of the DNA oligonucleotide;
(4) contacting the adapter oligonucleotide with a fluorescent oligonucleotide (i.e., an immunoFISH fluorescent oligonucleotide) capable of hybridizing with a second adapter sequence of the adapter oligonucleotide;
(5) imaging the at least one cell to detect the at least one fluorescent oligonucleotide, thereby generating immunoFISH image(s);
(6) reverse transcribing a barcode sequence that corresponds to a genetic perturbation in the cell;
(7) fixing the reverse transcribed barcode sequence, optionally via a PFA and/or glutaraldehyde treatment;
(8) hybridizing at least one padlock probe to padlock probe hybridization sequences flanking the reverse transcribed barcode sequence, connecting the ends of the at least one padlock probe (e.g., via gap filling), and amplifying the reverse transcribed barcode sequence, thereby generating barcode sequence amplicons;
(9) analyzing the phenotype of the cell (e.g., RNA foci/RNA aggregation imaging, antibody-DNA conjugate imaging, label-free imaging, high content imaging, calcium imaging, immunohistochemistry, cell morphology imaging, protein aggregation imaging, cell-cell interaction imaging, live cell imaging, or any other imaging-based assay modality (e.g., CellPaint));
(10) sequencing the barcode sequence in situ (e.g., via sequencing-by-synthesis), thereby generating POSH image(s);
(11) segmenting a morphological image of the cell to detect the plurality of cells and identify the boundaries of the plurality of cells in the morphological image; and,
(12) processing and/or transforming the images (e.g., the immunoFISH image(s), the POSH image(s), and/or the segmentation image(s)) using computational analysis to obtain images of the same cell with different readouts.

In some embodiments, the method is performed in numerical order of steps 1-12. In some embodiments, the method is performed out of numerical order of steps 1-12. In some embodiments, the method comprises steps in addition to steps 1-12. In some embodiments, the method comprises omitting one or more of steps 1-11.

In some embodiments, the method comprises the following steps:
(1) hybridizing a 3′ loop probe and a 5′ probe or a 5′ loop probe and a 3′ probe (i.e., RNA FISH probes; such as SNAIL probes) to a target sequence of an mRNA transcript in a cell;
(2) connecting the ends of the 3′ loop probe or the 5′ loop probe (such as by ligation) to form a circular probe;
(3) amplifying the mRNA target sequence (such as by RCA), thereby generating an DNA amplicon;
(4) contacting at least one cell with a conjugate comprising an antibody that specifically binds at least one protein of interest (i.e., an immunoFISH primary antibody) and a DNA oligonucleotide comprising a protein identification sequence uniquely associated with the protein of interest;
(5) contacting the conjugate with an adapter oligonucleotide (i.e., an immunoFISH adapter oligonucleotide), wherein the adapter oligonucleotide comprises a first adapter sequence complementary to a portion of the DNA oligonucleotide;
(6) contacting the adapter oligonucleotide with a fluorescent oligonucleotide (i.e., an immunoFISH fluorescent oligonucleotide) capable of hybridizing with a second adapter sequence of the adapter oligonucleotide;
(7) imaging the at least one cell to detect the at least one fluorescent oligonucleotide, thereby generating immunoFISH image(s);
(8) fixing the DNA amplicon in place, such as by bis-MHS-PEG exposure;
(9) reverse transcribing a barcode sequence that corresponds to a genetic perturbation in the cell;
(10) fixing the reverse transcribed barcode sequence, optionally via a PFA and/or glutaraldehyde treatment;
(11) detecting (e.g., imaging) the DNA amplicon, thereby generating an RNA FISH image;
(12) hybridizing at least one padlock probe to padlock probe hybridization sequences flanking the reverse transcribed barcode sequence, connecting the ends of the at least one padlock probe (e.g., via gap filling), and amplifying the reverse transcribed barcode sequence, thereby generating barcode sequence amplicons;
(13) analyzing the phenotype of the cell (e.g., RNA foci/RNA aggregation imaging, antibody-DNA conjugate imaging, label-free imaging, high content imaging, calcium imaging, immunohistochemistry, cell morphology imaging, protein aggregation imaging, cell-cell interaction imaging, live cell imaging, or any other imaging-based assay modality (e.g., CellPaint));
(14) sequencing the barcode sequence in situ (e.g., via sequencing-by-synthesis), thereby generating POSH image(s);
(15) segmenting a morphological image of the cell to detect the plurality of cells and identify the boundaries of the plurality of cells in the morphological image; and,
(16) processing and/or transforming the images (e.g., the RNA FISH image(s), the immunoFISH image(s), the POSH image(s), and/or the segmentation image(s)) using computational analysis to obtain images of the same cell with different readouts.

In some embodiments, the method is performed in numerical order of steps 1-16. In some embodiments, the method is performed out of numerical order of steps 1-16. In some embodiments, the method comprises steps in addition to steps 1-16. In some embodiments, the method comprises omitting one or more of steps 1-16.

In some embodiments, the method comprises the following steps:
(1) hybridizing a 3′ loop probe and a 5′ probe or a 5′ loop probe and a 3′ probe (i.e., RNA FISH probes; such as SNAIL probes) to a target sequence of an mRNA transcript in a cell;
(2) connecting the ends of the 3′ loop probe or the 5′ loop probe (such as by ligation) to form a circular probe;
(3) amplifying the mRNA target sequence (such as by RCA), thereby generating an DNA amplicon;
(4) contacting at least one cell with a first antibody which specifically binds at least one protein of interest (i.e., an immunoFISH primary antibody) and a DNA oligonucleotide comprising a protein identification sequence uniquely associated with the protein of interest;
(5) contacting the antibody with a conjugate comprising an antibody which specifically binds the first antibody and a DNA oligonucleotide comprising a protein identification sequence uniquely associated with the protein of interest;
(6) contacting the conjugate with an adapter oligonucleotide (i.e., an immunoFISH primary antibody), wherein the adapter oligonucleotide comprises a first adapter sequence complementary to a portion of the DNA oligonucleotide;
(7) contacting the adapter oligonucleotide with a fluorescent oligonucleotide (i.e., an immunoFISH fluorescent oligonucleotide) capable of hybridizing with a second adapter sequence of the adapter oligonucleotide;
(8) imaging the at least one cell to detect the at least one fluorescent oligonucleotide, thereby generating immunoFISH image(s);
(9) fixing the DNA amplicon in place, such as by bis-MHS-PEG exposure;
(10) reverse transcribing a barcode sequence that corresponds to a genetic perturbation in the cell;
(11) fixing the reverse transcribed barcode sequence, optionally via a PFA and/or glutaraldehyde treatment;
(12) detecting (e.g., imaging) the DNA amplicon, thereby generating an RNA FISH image;
(13) hybridizing at least one padlock probe to padlock probe hybridization sequences flanking the reverse transcribed barcode sequence, connecting the ends of the at least one padlock probe (e.g., via gap filling), and amplifying the reverse transcribed barcode sequence, thereby generating barcode sequence amplicons;
(14) analyzing the phenotype of the cell (e.g., RNA foci/RNA aggregation imaging, antibody-DNA conjugate imaging, label-free imaging, high content imaging, calcium imaging, immunohistochemistry, cell morphology imaging, protein aggregation imaging, cell-cell interaction imaging, live cell imaging, or any other imaging-based assay modality (e.g., CellPaint));
(15) sequencing the barcode sequence in situ (e.g., via sequencing-by-synthesis), thereby generating POSH image(s);
(16) segmenting a morphological image of the cell to detect the plurality of cells and identify the boundaries of the plurality of cells in the morphological image; and,
(17) processing and/or transforming the images (e.g., the RNA FISH image(s), the immunoFISH image(s), the POSH image(s), and/or the segmentation image(s)) using computational analysis to obtain images of the same cell with different readouts.

In some embodiments, the method is performed in numerical order of steps 1-17. In some embodiments, the method is performed out of numerical order of steps 1-17. In some embodiments, the method comprises steps in addition to steps 1-17. In some embodiments, the method comprises omitting one or more of steps 1-17.

In some embodiments, the method comprises the following steps:
(1) contacting RNA foci with a fluorescent oligonucleotide probe (i.e., an RNA foci FISH probe), wherein each fluorescent oligonucleotide probe comprises a target hybridization sequence complementary to a portion of a sequence in the RNA foci; and
(2) detecting the fluorescent oligonucleotide probe by imaging, thereby generating RNA foci FISH image(s);
(3) reverse transcribing a barcode sequence that corresponds to a genetic perturbation in the cell;
(4) fixing the reverse transcribed barcode sequence, optionally via a PFA and/or glutaraldehyde treatment;
(5) hybridizing at least one padlock probe to padlock probe hybridization sequences flanking the reverse transcribed barcode sequence, connecting the ends of the at least one padlock probe (e.g., via gap filling), and amplifying the reverse transcribed barcode sequence, thereby generating barcode sequence amplicons;
(6) analyzing the phenotype of the cell (e.g., RNA foci/RNA aggregation imaging, antibody-DNA conjugate imaging, label-free imaging, high content imaging, calcium imaging, immunohistochemistry, cell morphology imaging, protein aggregation imaging, cell-cell interaction imaging, live cell imaging, or any other imaging-based assay modality (e.g., CellPaint));
(7) sequencing the barcode sequence in situ (e.g., via sequencing-by-synthesis), thereby generating POSH image(s);
(8) segmenting a morphological image of the cell to detect the plurality of cells and identify the boundaries of the plurality of cells in the morphological image; and,
(9) processing and/or transforming the images (e.g., the RNA foci FISH image(s), the POSH image(s), and/or the segmentation image(s)) using computational analysis to obtain images of the same cell with different readouts.
   In some embodiments, the method is performed in numerical order of steps 1-9. In some embodiments, the method is performed out of numerical order of steps 1-9. In some embodiments, the method comprises steps in addition to steps 1-9. In some embodiments, the method comprises omitting one or more of steps 1-9.

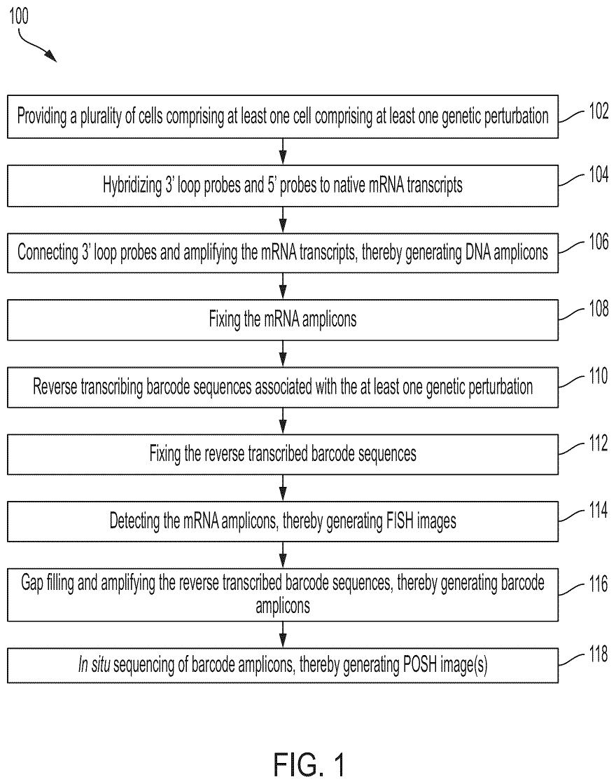

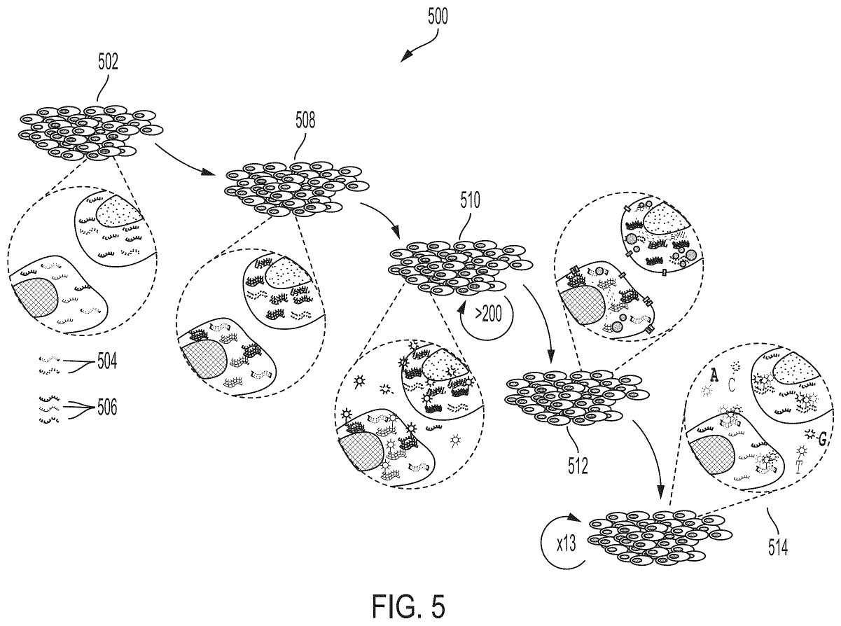

FIG. 1 illustrates an exemplary workflow process 100 for certain methods described herein. This unified workflow allows for at least RNA FISH and POSH assays to be performed on the same cell. In various embodiments of process 100, some blocks are combined. In some embodiments, the order of some blocks is changed. In some embodiments, some blocks are omitted. In some embodiments, the process 100 is performed in the order depicted in FIG. 1. In some examples, additional steps may be performed in the process 100. Accordingly, the operations as illustrated (and described in greater detail below) are exemplary by nature and, as such, should not be viewed as limiting. The process 100 is described below and is additionally illustrated in FIG. 5, which illustrates an exemplary schematic for certain methods described herein. At block 102 of FIG. 1, a plurality of cells comprising at least one cell comprising at least one genetic perturbation is provided. The genetic perturbation may be identified during downstream analysis by sequencing (such as by in situ sequencing-by-synthesis) a corresponding barcode sequence. At 104, 3′ loop probes and 5′ probes (i.e., FISH probes for RNA FISH) are hybridized to native mRNA transcripts in the cell comprising the genetic perturbation. In some embodiments, the mRNA transcripts comprise mRNA transcripts associated with a control or standard gene, such as GAPDH. In some embodiments, the mRNA transcripts comprise mRNA transcripts associated with a disease phenotype, which can be used to assess the effect of the genetic perturbation on the disease phenotype. At 106, the ends of the 3′ loop probes are connected (e.g., ligated) to form circular probes. The circular probes are amplified, such as by RCA, thereby generating DNA amplicons of the mRNA transcripts.

At 108, the DNA amplicons are fixed at their location, such as by bis-MHS-PEG exposure. At 110, the barcode sequence corresponding to the genetic perturbation is reverse transcribed, thereby generating a reverse transcribed barcode sequence. Following reverse transcription, at 112, the reverse transcribed barcode sequences are fixed at their location, such as by PFA. At 114, the DNA amplicons are detected (such as by multiple rounds of FISH imaging), thereby generating one or more FISH images. Padlock probes (e.g., at least one padlock probe) are then hybridized to padlock probe hybridization sequences flanking the reverse transcribed barcode sequence, followed by gap filling, to allow for amplification of the barcode sequence using RCA, thereby generating barcode amplicons, at 116. Finally, at 118, the barcode sequences (barcode amplicons) are sequenced in situ. The in situ sequencing optionally comprises sequencing-by-synthesis. POSH images may be generated during in situ sequencing. In some embodiments, the process includes a further immunoFISH method, in addition to POSH. In some embodiments, the process includes a further immunoFISH method, in addition to RNA FISH. In some embodiments, the process includes a further RNA foci FISH method, in addition to RNA FISH. In summary, the method allows for the matching of genetic perturbations to optical phenotypes, with simultaneous transcriptional measurements.

In order to obtain the images of the same cell with different readouts across multiple rounds of imaging (e.g., matching the images), the images can be processed such that a system can associate each cell with a cell identifier, a portion of each FISH image, and the corresponding barcode sequence associate with a genetic perturbation. The methods provided herein may comprise computational and machine learning methods for transforming and overlaying images with different readouts across multiple rounds of imaging, which are further described herein.

### Exemplary Preparation Process

An exemplary method of preparing samples for a screen according to the unified workflow is provided:
(1) Genes of interest are selected. A CRISPR library and corresponding gRNAs are designed from the selected genes.
(2) The gRNA library is synthesized and cloned into a lentiviral backbone containing padlock sequences flanks.
(3) Genes are selected to be measured in the transcriptional-readout portion of the experiment.
(4) Dual probes are made (3′ and 5′) for the target RNA site, which, in some embodiments, are designed for SNAIL to allow for rolling circle amplification of double-bound target sequences.
(5) Adapters are designed matching a gene ID to that located on the 3′ probes described in step (4), a toehold bridging sequence, and a fluorescent oligo-hybridization sequence.
(6) Fluorescent protein-binding oligonucleotides that can hybridize with the adapter described in (5) are designed.
(7) Toehold oligonucleotides that match the gene ID and toehold sequences described in step (4) are designed.
(8) The probes designed above are reviewed and confirmed to allow for multiple target RNAs to be simultaneously imaged (each matching a unique fluorophore) across multiple FISH cycles.
(9) Cas9-expressing cell cultures (such as IPSCs, cancer lines, primary cells, or the like) are infected with the lentiviral gRNA pool. Alternatively, in some embodiments, Cas9 and gRNA are provided using an all-in-one CRISPR/Cas system.
(10) The cellular experiments are performed, including live imaging, chemical treatment, exposures, and the like, to a target timepoint.
(11) The cell cultures are collected for processing according to the methods described herein.

In the exemplary method of preparing samples, steps (1), (2) and (9) involve preparation of the plurality of cells comprising at least one cell comprising at least one genetic perturbation. In this example, lentiviral vectors encoding gRNAs are used to infect Cas9-expressing cell cultures to provide the genetic perturbation (for example, a gene knockout/knockdown or novel or increased expression of a gene). The genetic perturbation preferably provides a disease model phenotype, which can be assessed in step (10), such as by induction of the disease phenotype, treatment of the disease phenotype, exacerbation of the disease phenotype, or the like. In some embodiments, the cells infected in step (9) are not Cas9-expressing cells. Rather, Cas9 can be provided by the lentiviral vector or by another vector. In step (3), genes are selected for assessment in the RNA FISH portion of the unified workflow described herein. Probes, adapters, and toehold oligonucleotides are designed/selected in steps (4)-(7). At step (8), which may be optional in some embodiments, the probes for RNA FISH are assessed for compatibility with the unified workflow, including to allow for multiple target RNAs to be simultaneously imaged across multiple RNA FISH cycles. This assessment can be experimental (such as by using the probes in an RNA FISH experiment in cells expressing the target mRNA transcripts across one or more cycles of RNA FISH) or computationally. At step (11), the cell cultures are collected for processing and assessment by the unified workflow described herein, including by RNA FISH, POSH, and other assessments including, in some embodiments, immunoFISH and/or RNA foci FISH.

Exemplary immunoFISH/RNA FISH/POSH Method (I)

An exemplary method of processing the cells described in the immediately preceding section in the unified workflow is provided:
(1) Samples are fixed via paraformaldehyde and optionally by preservation of protein and RNA by 70% ethanol at −20° C.
(2) Samples are permeabilized by ethanol and ethanol is then gradually removed by serial dilutions.
(2.5) If the unified workflow includes RNA foci FISH, then nonspecific DNA binding is blocked by addition of SNAIL probe binding buffer (with formamide and sheared salmon sperm DNA) for 1 hour at 55° C.
(3) If using RNA FISH, 3′ and 5′ SNAIL probes are hybridized to target RNA by overnight incubation at, for example, 40° C. with 300 rpm agitation (or, in some embodiments, 55° C. for if using RNA foci FISH).
(4) If using RNA FISH, thorough washing is performed followed by ligation by T4 DNA ligase to close the 3′ probe loop for 2 hours at room temperature under agitation. Thorough mixing after ligation.
(5) Rolling circle amplification of the RNA-labeling probe loops is performed.
(6) A blocking solution containing 1% nuclease-, protease-, and fatty acid-free BSA, 1 PBS buffer, 0.01% sodium azide, 0.1% Triton X 100, and 100 μg/ml sheared salmon sperm DNA (i.e., AbDil with salmon sperm DNA) is added and incubated for at least 1 hour at room temperature. Ribolock/RNAse inhibitor can be added optionally.
(7) If using streptavidin-biotin version of immunoFISH, a biotinylated DNA barcode is conjugated to a streptavidin-antibody conjugate in 1×PBS for at least 30 minutes at room temperature.
(8) If using immunoFISH, primary antibody-DNA conjugates or unconjugated primary antibodies are added to cells in AbDil with salmon sperm DNA overnight at 4° C. Ribolock/RNAse inhibitor can be added optionally during this step.
(9) If using immunoFISH, and if unconjugated primary antibody was used in (8), then fluorescently-tagged secondary antibodies are added in AbDil with salmon sperm DNA for at least 1 hour at room temperature.
(10) If using immunoFISH, multiple rounds of immunoFISH are performed targeting RNA biomarkers and/or proteins of interest are performed, with each round, in some embodiments, as follows: (a) fluorescent oligonucleotides and adapter oligonucleotides are added and allowed to anneal, (b) fluorescently-tagged adapters are hybridized to immunoFISH antibody-conjugated DNA barcodes, (c) the cells are imaged via fluorescence, and (d) toehold displacement of hybridized labels is performed.
(11) SNAIL amplicons are fixed by bis-MHS-PEG exposure at room temperature for 30 minutes.
(12) gRNA padlock reverse transcription (RT) primers are hybridized followed by fixation with glutaraldehyde and PFA fixing for 30 minutes at room temperature.
(13) Reverse transcription is performed with addition of reverse transcriptase and RT primers in appropriate buffer overnight at 37° C.
(14) Paraformaldehyde/glutaraldehyde fixation or other suitable fixation of samples for 30 minutes at room temperature is performed.
(15) Multiple rounds of RNA FISH are performed, with each round, in some embodiments, as follows: (a) fluorescent oligonucleotides and adapter oligonucleotides are added and allowed to anneal, (b) fluorescently-tagged adapters are hybridized to RCA-amplified target RNA sites, (c) cells are imaged by fluorescence (preferably by confocal microscopy), (d) toehold displacement is used to displace hybridized labels.
(16) Once the rounds of FISH (targeting RNA) are completed, samples undergo gap fill, such as by TaqIt and Ampligase.
(17) Padlocked sequences are amplified by rolling circle amplification at 30° C. overnight.
(18) Samples are then imaged using cellular dyes, brightfield microscopy, quantitative phase contrast, and/or antibody staining to derive morphological phenotypes.
(19) Samples are then primed for gRNA sequencing by synthesis for 15 minutes at 37° C.
(20) Samples then undergo several steps of in situ sequencing, such as, in some embodiments: (a) a wash, (b) addition of pooled fluorescently-tagged base pairs with polymerase in appropriate buffer, with 60° C. incubation for 3 minutes with agitation, (c) multiple rounds of washing (such as 4 rounds) followed by 60° C. incubation for 3 minutes with agitation, (d) step (c) is repeated, (e) DNA stain is added, such as low concentration Hoechst (such as 1:200,000 dilution), (f) samples are imaged, such as by widefield fluorescence, to capture the base pair of the gRNA at cycle X, (g) samples are washed, (h) stripping reagent is added for 6 minutes at 60° C., (i) step (h) is repeated, (k) one more wash at large volume, followed by incubation at 60° C. for 5 minutes.

In the above exemplary protocol describing a unified workflow of immunoFISH, FISH, and POSH, the order and manner of steps was found to facilitate the unified workflow by focusing on amplifying, RTing, and/or fixing the most rapidly-degrading components as quickly as possible. FISH readouts measured in step (15), the morphological/protein measurements in steps (10) and (18), and the multiple steps of sequencing in step (20) can be combined to form simultaneous transcriptional, proteomic, and perturbation (gRNA) information for all cells within the pool. Steps (3) to (11) amplify the native mRNA and RT/fix the barcoded gRNAs, ensuring that the information provided by these sensitive molecules is preserved and stabilized in the cells for downstream analysis and processing. This enables the cyclic imaging and other downstream steps to be conducted without risk of degradation of signal. Steps (6)-(10) of the exemplary process use AbDil with 1% BSA to block nonspecific protein binding. In some embodiments, other blocking solutions that come with detergents are used, so long as they include an agent to block nonspecific DNA binding, such as sheared salmon sperm DNA. The timing of steps (6), (8), and (9) may be, in some embodiments, adjusted (such as from 1 hour to overnight) and the temperature may be, in some embodiments, adjusted (such as from 4° C. to room temperature), such as to optimize the protocol based on the particular antibody being used. Whether step (7) is performed depends upon the type of conjugation chemistry that was used to link the immunoFISH DNA barcode with its antibody. If the DNA barcode was directly conjugated to the antibody already, there is no need for the extra conjugation step of step (7). Whether step (9) is performed depends upon whether immunoFISH or regular antibody staining is being used. If any non-fluorescent/non-immunoFISH antibodies are used, they cannot be read out with fluorescent oligonucleotides in step (10), so binding a fluorescent secondary antibody would be necessary. Secondary binding can also be performed at various points in the procedure (such as after step (11), after step (14), after step (15), and/or after step (17) with lower antibody signal. The probe binding and washing step for RNA foci FISH is very similar to the probe binding and washing step for FISH (step (3)) but typically at a higher temperature (55° C.) with an additional blocking step (step (2.5)) and without the ligation and amplification steps (steps (4)-(5)) used in SNAIL. The blocking procedure for RNA foci FISH is performed in the same buffer as probe binding, which is compatible with POSH. Step (11), in which SNAIL amplicons are stabilized by treatment with bis-MHS-PEG, was found to allow the amplicons to remain stationary during the various following steps that fix/amplify the gRNA probes. Drifting or loss of SNAIL amplicons reduces data quality significantly. Step (12) was found to improve POSH outcome. Step (12) provides hybridization of reverse transcription primers and an additional fixation, such as with glutaraldehyde and formaldehyde for 30 minutes at room temperature. It was found that both of these parts of step (12) improved the POSH outcome of the method (by increasing the number of POSH dots per cell as well as the intensity of those dots, which corresponds to number of RCA repeats; see Example 2 herein). Hybridization is performed in step (12) followed by the fixation of step (12). After fixation, step (13) involves replacing the fixation reagents with RT enzyme mix for reverse transcription at 37° C. for a period of time (such as overnight). The RT enzyme mix contains additional RT primers. Step (20b) involves the addition of fluorescently tagged base pairs and polymerase in suitable buffer followed by incubation, which allows for those base pairs to be incorporated into the sequencing primer bound to the POSH guide sequence. The fluorescently tagged base pairs include, for example, reversible terminators, so that only 1 base can be incorporated into the sequencing primer per POSH sequencing cycle. The base is imaged after incorporation and stripping mix is added in step (20h), which includes reagents to cleave both the fluorescent tag and the base terminator such that a new base can be added by polymerase in the next POSH sequencing cycle without signal from previous cycles.

### Cells and Genetic Perturbations

A variety of cell types are compatible with the provided methods. The cells may be genetically edited or otherwise modified or may comprise a natural genetic variation, particularly and preferably one associated with a disease model phenotype.

In some embodiments, the cells comprise a natural (e.g., endogenous) genetic variation. For example, the cells used in the methods provided herein may comprise a mixture of various cell lines such that there is a natural genetic variation between the cells.

The cells may be genetically edited in order to perform a genetic screen. In some embodiments, the cells are genetically edited (e.g., comprise a genetic perturbation). For example, the cells may be engineered to comprise one or more genetic perturbations. In some embodiments, the cells comprise a CRISPR system, such as a CRISPR interference (CRISPRi) or CRISPR activation (CRISPRa) system. In some embodiments, the cells are engineered to constitutively express a Cas protein, such as Cas9, enabling them to be used for genetic screening experiments. In some embodiments, a plurality of cells are engineered to comprise a plurality of genetic perturbations, such that certain individual cells within the plurality of cells comprise at least one genetic perturbation. The plurality of cells may then be assessed by the various methods described herein to link the genetic perturbation with the phenotype of the corresponding cell.

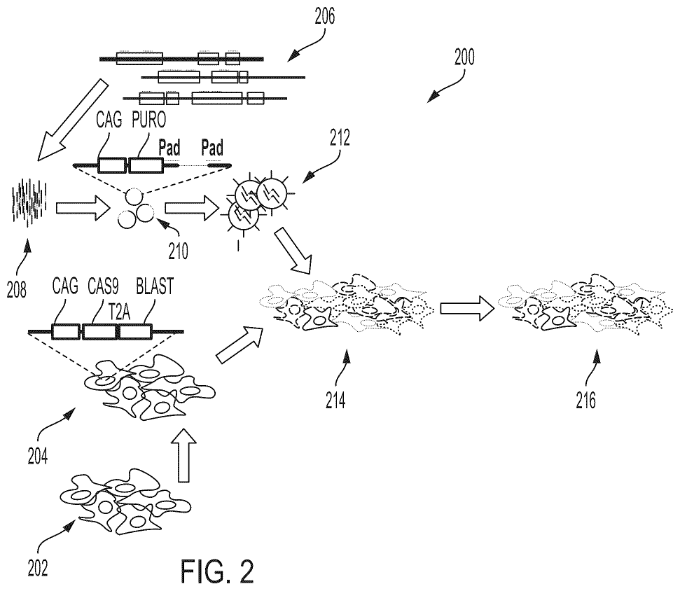

FIG. 2 illustrates an exemplary process 200 for generating a plurality of genetically edited cells, in accordance with some embodiments. A plurality of cells 202 are engineered to express Cas9 at 204. The plurality of cells may comprise any suitable cell type, including, but not limited to, induced pluripotent stem cells (iPSCs), differentiated iPSCs, tumor cells, or primary cells. At 206, genes are selected for targeting (i.e., as targets for the genetic perturbation) and guide RNAs (gRNAs) targeting said genes are designed from the given genes. The gRNA library is synthesized at 208 and cloned into vectors (such as lentiviral vectors) comprising padlock probe hybridization sequences (“Pad” in FIG. 2) flanking the inserted gRNA 210. During downstream processing, the padlock probe hybridization sequences are used to amplify and subsequently identify the gRNAs they flank, such as during pooled optical genetic screening (POSH) in situ sequencing. Expression of the gRNA may be controlled by a promoter, such as the CAG promoter (“CAG” in FIG. 2). The lentiviral vector may additionally comprise an antibiotic resistance gene, such as one for puromycin (“PURO” in FIG. 2), to allow for the selection of cells that have been successfully transfected with the lentiviral vector comprising the gRNA.

Once the lentiviral vectors are engineered and express the gRNA at 212, the vectors are transfected into the previously described plurality of cells expressing Cas9 204, at 214, to generate a genetically engineered plurality of cells. The delivered gRNA associates with the Cas9, producing double-stranded breaks at the locations specified by the gRNA, leading to a plurality of genetically engineered cells (i.e., cells comprising at least one genetic perturbation). At least one cell of the plurality of cells has had a gene knocked out while expressing the padlock probe hybridization sequence flanked gRNAs that act as a ‘barcode sequence’ identifying the edited gene.

Finally, at 216 the cells may undergo downstream analysis that may include, but is not limited to, live cell imaging (e.g., quantitative phase imaging), fluorescence in situ hybridization (FISH; such as RNA FISH, immunoFISH, and/or RNA foci FISH), calcium imaging, and/or determination of protein aggregation and transfer, lysosomal accumulation, RNA trafficking, phagocytosis, cell-cell interactions, and cell migration, or any combination thereof. POSH may be used to identify the barcode which identifies the genetic perturbation of each cell.

### Cells and Cell Culture

In some embodiments, the method comprises providing a plurality of cells. In some embodiments, each cell of the plurality of cells are each of the same type. In some embodiments, the plurality of cells comprises different types of cell populations within the plurality of cells. For example, cells may be primary cells, stem cells, iPSCs, iPSC-derived stellate cells (iStels), cancer cells (e.g., immortalized cancer cells), primary tissue biopsies, or any combination thereof.

In some embodiments, the cells are stem cells. In some embodiments, the stem cells are pluripotent stem cells. In some embodiments, the cells are iPSCs. In some embodiments, iPSCs are generated from somatic cells by introducing one or more known reprogramming factors. In some embodiments, the iPSCs are differentiated prior to performing a method described herein. Stem cells are characterized by their ability to renew themselves through division of mitotic cells and differentiation into a diverse range of specialized cell types. There are two major types of mammalian stem cells: embryonic stem cells found in blastocysts and adult stem cells found in adult tissues. In developing embryos, stem cells can differentiate into any specialized embryonic tissue. In adults, stem and progenitor cells function as a repair system for the body, not only recruiting specialized cells, but also maintaining normal turnover of regenerative organs such as blood, skin, or intestinal tissue. Human embryonic stem cells (hES) can be defined by the presence of several transcription factors and cell surface proteins. Transcription factors Oct4, Nanog, and Sox2 form a core regulatory network that reliably represses genes that lead to the maintenance of differentiation and pluripotency. The cell surface antigens most often used to identify hES cells include glycolipids SSEA3 and SSEA4 and keratan sulfate antigens Tra-1-60 and Tra-1-81.

The generation of iPSCs, as is known in the art, depends on the gene or genes used for induction. Factors such as Oct3/4, KLF4, Sox2 and/or c-myc or combinations thereof can be used. Nucleic acids encoding these reprogramming factors can be included in monocistronic or multicistronic expression cassettes. Similarly, the nucleic acid encoding the monocistronic or multicistronic expression cassette can be included in one reprogramming vector or multiple reprogramming vectors.

iPSCs are typically generated by transfecting specific stem cell-related genes into non-pluripotent cells such as adult fibroblasts or cord blood cells. Transfection can be accomplished with integrated viral vectors such as retroviruses (e.g., lentiviruses) or non-integrated viral vectors such as Sendai virus. Reprogramming may also be done using virus-free methods such as episomal reprogramming or mRNA reprogramming. After the critical period, a small number of transfected cells begin to resemble morphologically and biochemically to pluripotent stem cells can be separated based on morphological selection, doubling time, reporter gene expression, and/or antibiotic resistance.

Pluripotent cells can be cultured and maintained in an undifferentiated state using various methods. In some embodiments, matrix components may be included in a given medium to culture and maintain pluripotent cells in a substantially or essentially undifferentiated state. Various matrix components can be used to culture and maintain pluripotent cells such as hESCs or iPSCs. For example, collagen IV, fibronectin, laminin, and vitronectin may be used in combination to provide a solid support for embryonic cell culture and maintenance.

A matrix material may be used to provide a substrate for cell culture and maintenance of pluripotent cells. In some embodiments, the substrate is Matrigel™ (a gelatinous protein mixture secreted by mouse tumor cells and is commercially available from BD Biosciences (New Jersey, USA)). The mixture resembles the complex extracellular environment found in many tissues and is used by cell biologists as a substrate for cell culture. It will be appreciated that additional methods of culturing and maintaining iPSCs are well known to those of skill in the art and may be used with embodiments of the present invention.

In some embodiments, the cells comprise cells derived from primary cells obtained or isolated from one or more individual subjects or donors. In some embodiments, the cells are obtained from a patient's biopsy tissue.

In some embodiments, the method further comprises culturing the plurality of cells prior to analyzing the phenotype. In some embodiments, the culture medium used to culture the cells contains serum. In some embodiments, the culture medium used to culture the cells is serum-free. A serum-free medium refers to a medium that does not contain untreated or unpurified serum, and thus can include a medium having purified blood-derived components or animal tissue-derived components (e.g., growth factors). From the viewpoint of preventing contamination with different animal-derived components, the serum may be derived from the same animal as the cells. The culture medium may or may not contain a serum replacement. Serum substitutes include albumin (albumin substitutes such as lipid-rich albumin, recombinant albumin, plant starch, dextran and protein hydrolysates), transferrin (or other iron transporters), fatty acids, insulin, collagen precursors, trace elements, 2-mercaptoethanol, or 3′-thiolgiicerol, or an equivalent thereof.

In some embodiments, the plurality of cells is on a substrate or in a three dimensional culture. In some embodiments, the plurality of cells is on a substrate. In some embodiments, the substrate is any standard tissue culture container such as a tissue culture plate or flask. In some embodiments, the substrate is a cell culture dish. In some embodiments, the substrate is a tissue culture plate. In some embodiments, the substrate is a petri dish. In some embodiments, the substrate is a tissue culture flask. In some embodiments, the substrate is a well of a standard microwell plate, such as a 6-well, 12-well, 24-well, 96-well, 384-well, or 1,536-well plate. In some embodiments, the substrate is a 6-well plate. In some embodiments, the substrate is a 12-well plate. In some embodiments, the substrate is a 24-well plate. In some embodiments, the substrate is a 96-well plate. In some embodiments, the substrate is a 384-well plate. In some embodiments, the substrate is a 1,536-well plate. The substrate may be made of any material for imaging using the imaging modalities described herein. In certain embodiments, the plate may be plastic-bottom plates suitable for imaging using the imaging modalities described herein. In certain embodiments, the plate may be glass-bottom plates suitable for imaging using the imaging modalities described herein. In certain embodiments, the substrate may be a culture chamber in an array of culture chambers defined on a microfluidic device, or droplet generated on a microfluidic device.

In certain example embodiments, a plurality of cells may be cultured on individual microscopic slides in culture medium. In some embodiments, the plurality of cells is in three-dimensional culture. In some embodiments, the three-dimensional culture comprises a scaffold. In some embodiments, the scaffold comprises a three-dimensional matrix. In some embodiments, the three-dimensional matrix comprises a material selected from the group consisting of BD Matrigel™ basement membrane matrix (BD Sciences), Cultrex® basement membrane extract (BME; Trevigen), hyaluronic acid, polyethylene glycol (PEG), polyvinyl alcohol (PVA), polylactide-co-glycolide (PLG), and polycaprolactone (PLA). In some embodiments, the three-dimensional matrix comprises BD Matrigel™ basement membrane matrix (BD Sciences). In some embodiments, the three-dimensional matrix comprises Cultrex® basement membrane extract (BME; Trevigen). In some embodiments, the three-dimensional matrix comprises hyaluronic acid. In some embodiments, the three-dimensional matrix comprises polyethylene glycol (PEG). In some embodiments, the three-dimensional matrix comprises polyvinyl alcohol (PVA). In some embodiments, the three-dimensional matrix comprises polylactide-co-glycolide (PLG). In some embodiments, the three-dimensional matrix comprises polycaprolactone (PLA). In some embodiments, the three-dimensional culture is scaffold-frec.

In some embodiments, at least one cell of the plurality of cells comprises a CRISPR system. In some embodiments, the CRISPR system is a CRISPR interference (CRISPRi) system). In some embodiments, the CRISPR system is a CRISPR activation (CRISPRa) system. In some embodiments, at least one cell of the plurality of cells expresses or comprises a Cas protein. In some embodiments, a Cas protein comprises a core Cas protein. Exemplary Cas core proteins include, but are not limited to Cas1, Cas2, Cas3, Cas4, Cas5, Cas6, Cas7, Cas8 and Cas9. In some embodiments, the Cas protein is Cas12a. In some embodiments, the Cas protein is Cas13. In some embodiments, the Cas protein is Cas9. In some embodiments, a Cas protein comprises one or more amino acid substitutions or modifications. In some embodiments, the substitutions and/or modifications can prevent or reduce proteolytic degradation and/or extend the half-life of the polypeptide in a cell.

### Perturbations and Genetic Barcodes

In some embodiments, the methods provided herein comprise providing a plurality of cells comprising at least one cell comprising at least one genetic perturbation. In some embodiments, the methods further comprise generating the plurality of cells comprising at least one cell comprising the at least one genetic perturbation. The genetic perturbation is identified by a barcode sequence associated with the genetic perturbation. For example, when a gRNA is used with a CRISPR-Cas system to introduce the genetic perturbation into the cell, the gRNA comprises a barcode sequence thereby identifying the genetic perturbation. In some embodiments, the method further comprises introducing a genetic perturbation and its corresponding barcode sequence into at least one cell of a plurality of cells.

In some embodiments, the genetic perturbation of a cell occurs using a CRISPR/Cas system. Any CRISPR/Cas system that is capable of altering a target polynucleotide sequence in a cell can be used. These CRISPR-Cas systems can employ a variety of Cas proteins (Haft et al. PLOS Comput Biol. 2005; 1 (6)e60), which are described herein. The machinery of the Cas protein that enables the CRISPR/Cas system to alter target polynucleotide sequences in cells includes RNA binding proteins, endo- and exo-nucleases, helicases, and polymerases. In some embodiments, the CRISPR/Cas system is a CRISPR type I system. In some embodiments, the CRISPR/Cas system is a CRISPR type II system. In some embodiments, the CRISPR/Cas system is a CRISPR type V system. In some embodiments, the CRISPR/Cas system is a CRISPRi system. In some embodiments, the CRISPR/Cas system is a CRISPRa system.

In some embodiments, the method comprises stably integrating the genetic perturbation and barcode sequence corresponding thereto into the genome of at least one cell. In some embodiments, the genetic perturbation and corresponding barcode sequence are delivered into the cell using a viral vector. In some embodiments, the viral vector is a retroviral vector. In some embodiments, the retrovirus is or is derived from a Moloney murine leukemia virus (MMULV), feline immunodeficiency virus (FIV), Harvey murine sarcoma virus (HaMuSV), murine mammary tumor virus (MuMTV), gibbon ape leukemia virus (GaLV), human immunodeficiency virus (HIV), Rous Sarcoma Virus (RSV), or lentivirus. In some embodiments, the virus is a lentivirus. In some embodiments, the virus is derived from a lentivirus. In some embodiments, the U3 sequence from the lentiviral 5′ LTR may be replaced with a promoter sequence in the viral construct. This may increase the titer of virus recovered from the packaging cell line. An enhancer sequence may also be included. In some embodiments, the virus encodes a selectable marker. In some embodiments, the selectable marker is an antibiotic resistance gene. In some embodiments, the antibiotic resistance gene confers resistance to an antibiotic selected from the group consisting of puromycin, hygromycin, bleomycin, neomycin, actinomycin D, and mitomycin C. In some embodiments, the antibiotic resistance gene confers resistance to puromycin. In some embodiments, the antibiotic resistance gene confers resistance to hygromycin. In some embodiments, the antibiotic resistance gene confers resistance to bleomycin. In some embodiments, the antibiotic resistance gene confers resistance to neomycin. In some embodiments, the antibiotic resistance gene confers resistance to actinomycin D. In some embodiments, the antibiotic resistance gene confers resistance to mitomycin C. In some embodiments, the virus encodes a fluorescent protein. In some embodiments, the fluorescent protein is selected from the group consisting of a green fluorescent protein, a red fluorescent protein, a blue fluorescent protein, a cyan fluorescent protein, a yellow fluorescent protein, and an orange fluorescent protein.

In some embodiments, the barcode sequence is a sequence of nucleotides (for example, DNA or RNA) that is used as an identifier for the genetic perturbation. A barcode may also refer to any nucleic acid sequence that may be used to identify the originating source of a nucleic acid fragment. The barcode sequence, in some embodiments, can be part of a contiguous polynucleotide sequence comprising the gRNA. In some embodiments, the gRNA itself is or contains the barcode sequence. In some embodiments, the gRNA itself, or a portion of the gRNA sequence, is part of the barcode sequence.

Target molecules can be optionally labeled with multiple barcode sequences in combinatorial fashion (for example, using multiple barcode sequences bound to one or more specific binding agents that specifically recognizing the target molecule), thus greatly expanding the number of unique identifiers possible within a particular barcode sequence pool. In certain embodiments, barcode sequences are added to a growing barcode concatemer attached to a target molecule, for example, one at a time. In other embodiments, multiple barcode sequences are assembled prior to attachment to a target molecule.

In some embodiments, each barcode sequence is at 3 to about 18 base pairs in length, such as about 3 to any of about 5, about 6, about 7, about 8, about 9, about 10, about 11, about 12, about 13, about 14, about 15, about 16, about 17, and about 18 base pairs in length. In some embodiments, each barcode sequence is 3 to about 12 base pairs in length. In some embodiments, each barcode sequence is 3 to about 10 base pairs in length. In some embodiments, each barcode sequence is 3 to about 8 base pairs in length. In some embodiments, each barcode sequence is 5 to about 12 base pairs in length. In some embodiments, each barcode sequence is 5 to about 10 base pairs in length. In some embodiments, each barcode sequence is 5 to about 8 base pairs in length. In some embodiments, each barcode sequence is 8 to about 12 base pairs in length. In some embodiments, each barcode sequence is 3 base pairs in length. In some embodiments, each barcode sequence is 4 base pairs in length. In some embodiments, each barcode sequence is 5 base pairs in length. In some embodiments, each barcode sequence is 6 base pairs in length. In some embodiments, each barcode sequence is 7 base pairs in length. In some embodiments, each barcode sequence is 8 base pairs in length. In some embodiments, each barcode sequence is 9 base pairs in length. In some embodiments, each barcode sequence is 10 base pairs in length. In some embodiments, each barcode sequence is 11 base pairs in length. In some embodiments, each barcode sequence is 12 base pairs in length. In some embodiments, each barcode sequence is 13 base pairs in length. In some embodiments, each barcode sequence is 14 base pairs in length. In some embodiments, each barcode sequence is 15 base pairs in length. In some embodiments, each barcode sequence is 16 base pairs in length. In some embodiments, each barcode sequence is 17 base pairs in length. In some embodiments, each barcode sequence is 18 base pairs in length.

In certain embodiments, the barcode sequence may be detected directly using an in situ sequencing method, as described herein. Because the barcode sequence is associated with the genetic perturbation of a particular cell, identifying (i.e., sequencing) the barcode sequence allows for the correlation of a particular genetic perturbation with a particular cell, thereby associating a genetic perturbation with the cell's transcriptome and/or morphology.

In some embodiments, the plurality cells are engineered using a guide RNA (gRNA) to generate the at least one genetic perturbation. In some embodiments, the method comprises contacting the plurality of cells with a gRNA library comprising a plurality of gRNAs to generate a plurality of genetic perturbations. Preferably, the gRNA library comprises a plurality of unique of gRNAs targeting a plurality of genomic targets. The gRNA library may then be used to engineer a plurality of cells to comprise a plurality of genetic perturbations. In a particular embodiment, the plurality of cells are engineered such that the individual cells within the plurality of cells comprise a median of one genetic perturbations each corresponding to a single gRNA. In some embodiments, the method comprises synthesizing the gRNA library. In some embodiments, the method comprises engineering the gRNA into a viral vector or the gRNA library into a library of viral vectors.

As described above, the gRNA library may be delivered to a plurality of cells via a viral vector. The plurality of cells may be engineered to transiently or constitutively express a Cas protein (such as Cas9) such that the introduction of a gRNA produces genetic perturbations across the plurality of cells. In some embodiments, the viral vectors comprise padlock probe hybridization sequences (e.g., a first padlock probe hybridization sequence and a second padlock probe hybridization sequence) in the vector. In some embodiments, the padlock probe hybridization sequences flank the gRNA in the vector. For example, the first padlock probe hybridization sequence may be immediately adjacent and upstream of the gRNA sequence in the vector and the second padlock probe hybridization sequence may be immediately adjacent and downstream of the gRNA sequence in the vector, or vice versa. In some embodiments, the padlock probe hybridization sequences are not immediately adjacent to the gRNA sequence, and are instead separated from the gRNA sequence by intervening nucleotides, such as 1, 2, 3, 4, or 5 intervening nucleotides. The padlock probe hybridization sequences enable the detection (e.g., sequencing) of the gRNA during downstream analysis. In some embodiments, the delivery of the padlock probe hybridization sequence flanked gRNA library to the plurality of cells generates cells with genetic perturbations (e.g., a gene knocked out) while expressing the padlock probe hybridization sequence flanked gRNAs (e.g., barcode sequences).

### Fluorescence In Situ Hybridization (FISH)

In situ hybridization methods are useful for analyzing a target nucleic acid in a cell or tissue sample. The methods provided herein comprise performing at least one round of fluorescence in situ hybridization (FISH, such as RNA FISH (such as SNAIL FISH), immunoFISH, and/or RNA foci FISH) on the plurality of cells. Different FISH methods are compatible with each other in the unified workflow described herein, including, for example, the combination of RNA FISH and immunoFISH, RNA FISH and RNA foci FISH, or all of RNA FISH, immunoFISH, and RNA foci FISH in the same unified workflow.

In some embodiments, the at least one round of RNA FISH comprises hybridizing a plurality of probes and/or probe sets (e.g., RNA FISH probes) to a plurality of mRNA transcripts, connecting the ends of the probes and/or probe sets to form a plurality of circular probes, amplifying a plurality of mRNA target sequences using the circular probes as templates to form a plurality of DNA amplicons, and detecting the DNA amplicons. The DNA amplicons are rolling circle amplicons, generated from mRNA target sequences using RNA FISH probes. In some embodiments, mRNA transcripts comprising mRNA target sequences may be identified and detected using any suitable methods or techniques, including those described herein, such as sequential fluorescent in situ hybridization (seqFISH), single-molecule fluorescent in situ hybridization (smFISH), multiplexed error-robust fluorescence in situ hybridization (MERFISH), in situ sequencing, targeted in situ sequencing, fluorescent in situ sequencing (FISSEQ), or spatially-resolved transcript amplicon readout mapping (STARmap). Sec, e.g., Enget et al. (2019) Nature 568, 235-239; Raj et al. (2008) Nat Methods. 5(10):877-9; Xia et al. (2019) PNAS 116.39: 19490-19499; Lee et al. (2015) Nat Protoc 10, 442-458; and. Wang, et al. Science (New York. N.Y.) vol. 361,6400 (2018): eaat5691, which are hereby incorporated by reference in their entirety.

A Splint Nucleotide Assisted Intramolecular Ligation (“SNAIL”) probe set (e.g., a 3′ loop probe and a 5′ probe or a 5′ loop probe and a 3′ probe) may be used to form a circular probe on an RNA molecule (i.e., an mRNA transcript). The circular probe is then amplified (such as by rolling circle amplification (RCA)) for in situ analysis and detection by imaging. See, e.g., U.S. Pat. Pub. 20190055594 and WO2019199579A1, which are hereby incorporated by reference in their entirety.

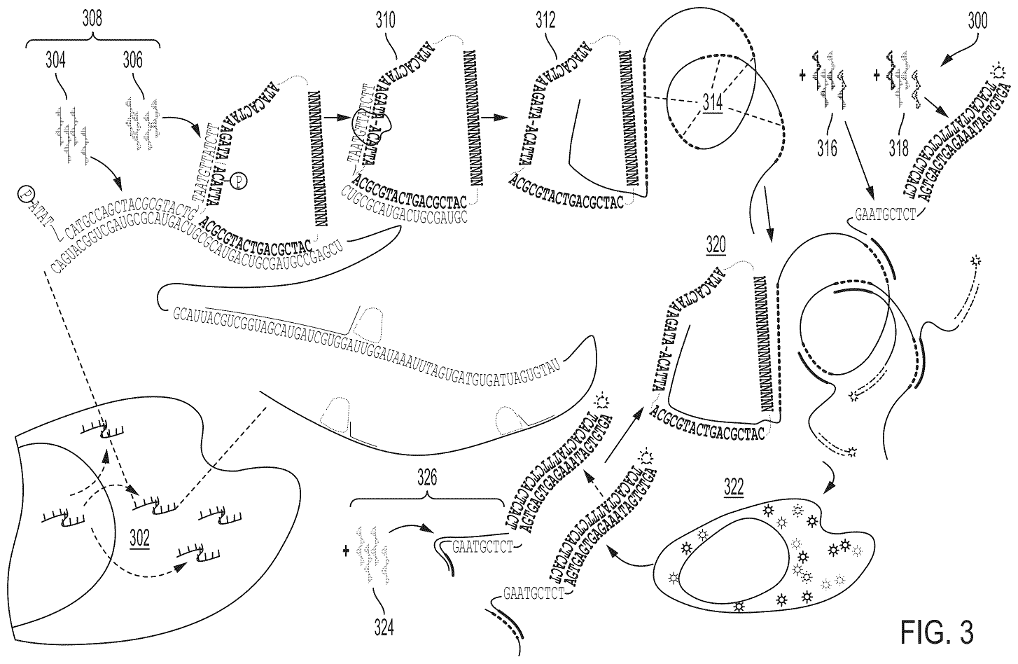

As illustrated in the exemplary process 300 of FIG. 3, a plurality of mRNA transcripts 302 from a plurality of cells are contacted with a plurality of 5′ probes 304 and a plurality of 3′ loop probes 306. The mRNA transcripts may have been previously identified as possible biomarkers of disease. The 5′ probes 304 and 3′ loop probes 306 hybridize with the mRNA transcripts 302 at 308, and the cells are subsequently washed to remove unhybridized probes. Following hybridization 308, the 3′ loop probes 306 are ligated using a T4 DNA ligase to close the loop, thereby generating circular probes 310. The circular probes are then amplified via RCA 312. Amplification of the circular probes generates DNA amplicons comprising multiple copies of a gene identifier (GID) sequence 314. The GID acts as a unique sequence which is amplified only at regions where both the 5′ and 3′ probes (i.e., the 5′ loop probe and 3′ probe, or the 3′ loop probe and 5′ probe) have bound to native mRNA to allow for amplification. Further, the DNA amplicons comprising the GIDs may be fixed at their location (such as by bis-MHS-PEG exposure). The position of the fixed DNA amplicons may then be recorded.

FISH imaging is then performed in cycles comprising steps 320, 322, and 326 according to FIG. 3. In each cycle, the DNA amplicons are contacted with adapter oligonucleotides 316 and detection probes 318 (e.g., fluorescent oligonucleotides), which are designed such that each DNA amplicon to be imaged has a single GID sequence 314 and single adapter oligonucleotide 316/detection probe 318 pair that binds with said GID sequence 314, enabling multiple colors (e.g., mRNA transcripts) to be imaged simultaneously. The adapter oligonucleotides 316 specifically hybridize to the detection probes 318, and additionally specifically hybridize with the GID sequences 314 of the DNA amplicons at 320. Samples are then imaged using a confocal microscope 322. To complete a FISH imaging cycle, the DNA amplicons are contacted with toehold displacement oligonucleotides 324 to release hybridized adapter oligonucleotide 316/detection probe 318 pairs from GID sequences 314 on the DNA amplicons 326. Steps 320, 322, and 326 may be, and generally would be, repeated (e.g., at least 2 times, such as anywhere between 2 and 100 times, and values and ranges there between) until the mRNA transcripts of interest (e.g., genes of interest) are imaged. In some embodiments, at least one unique mRNA transcript is imaged in each round. In some embodiments, a control mRNA transcript is imaged in one or more, or all, of the rounds.

The methods provided herein comprise performing at least one round of FISH (such as RNA FISH, immunoFISH, and/or RNA foci FISH). In some embodiments, the method comprises performing more than one round of FISH, such as any of 2, 3, 4, 5, 6, 7, 8, 9, 10, or more, rounds of FISH. For example, a sufficient number of RNA FISH rounds may be performed in order to detect the desired mRNA transcripts (e.g., genes of interest) within a plurality of cells. In each round of RNA FISH, more than one mRNA target may be assessed. In addition, a control mRNA may be assessed in each round. For example, in an exemplary embodiment, a first round of FISH may target four unique transcript products (i.e., four unique mRNA transcripts comprising mRNA target sequences) and a cell control mRNA (such as a housekeeping gene product, such as GAPDH), for a total of five unique mRNA targets in the first cycle. After assessing these five targets, a subsequent round of FISH may then assess four other unique transcript products and the cell control again.

### Probe Hybridization, Probe Connection, and Amplification

The at least one round of FISH, when using RNA FISH, comprises use of probes or a set of probes, for instance the use of two or more oligonucleotide probes (e.g., a 3′ loop probe and a 5′ probe or a 5′ loop probe and a 3′ probe). For example, the two or more oligonucleotide probes may be a SNAIL probe set. In some embodiments, the two or more probes comprise two different oligonucleotide sequences. The two or more probes may be used to analyze a target nucleic acid, e.g., a mRNA target sequence on an mRNA transcript in at least one cell of the plurality of cells, when they contact the cells. In some embodiments, the two or more probes may be provided to the cells at the same time, or each of the probes may be provided sequentially to the cells. The two or more probes are designed to facilitate the connection of the ends of one probe (e.g., a 3′ loop probe or a 5′ loop probe), to form a circular probe which may be amplified. In some embodiments, the probes are used to analyze, e.g., detect/image, an mRNA transcript or an DNA amplicon thereof.

The methods provided herein may comprise use of a probe that is a 3′ loop probe or a 5′ loop probe. In some embodiments, the 3′ loop probe or the 5′ loop probe comprises a first target hybridization sequence complementary to a first portion of an mRNA transcript of a plurality of mRNA transcripts. In some embodiments, the 3′ loop probe or the 5′ loop probe is between about 5 and 40 nucleotides in length, such as between about any of 5 and 15, 10 and 25, 15 and 30, 20 and 35, and 25 and 40 nucleotides in length. In some embodiments, the 3′ loop probe or the 5′ loop probe is greater than about 5 nucleotides in length, such as greater than about any of 10, 15, 20, 25, 30, 35, 40, or more, nucleotides in length. In some embodiments, the 3′ loop probe or the 5′ loop probe is less than about 40 nucleotides in length, such as less than about any of 35, 30, 25, 20, 15, 10, 5, or fewer, nucleotides in length. In some embodiments, the first target hybridization sequence of the 3′ loop probe is between about 5 and 40 nucleotides in length, such as between about any of 5 and 15, 10 and 25, 15 and 30, 20 and 35, and 25 and 40 nucleotides in length. In some embodiments, the first target hybridization sequence of the 3′ loop probe or the 5′ loop probe is greater than about 5 nucleotides in length, such as greater than about any of 10, 15, 20, 25, 30, 35, 40, or more, nucleotides in length. In some embodiments, the first target hybridization sequence of the 3′ loop probe or the 5′ loop probe is less than about 40 nucleotides in length, such as less than about any of 35, 30, 25, 20, 15, 10, 5, or fewer, nucleotides in length.

In some aspects, the 3′ loop probe or the 5′ loop probe contains a gene identifier (GID) sequence. The GID sequence may be used to identify the mRNA transcript to which the 3′ loop probe and 5′ probe or the 5′ loop probe and 3′ probe is hybridized. In some embodiments, the GID sequence is immediately adjacent to the first target hybridization sequence of the 3′ loop probe or the 5′ loop probe. In some embodiments, the GID sequence is not immediately adjacent to the first target hybridization sequence one another and instead are separated by a gap, for instance a gap of 1, 2, 3, 4, or 5 nucleotides. In some embodiments, the GID sequence is between about 5 and 20 nucleotides in length, such as between about any of 5 and 10, 8 and 15, 10 and 18, 15 and 20 nucleotides in length. In some embodiments, the GID sequence is greater than about 5 nucleotides in length, such as greater than about any of 10, 15, 20, or more, nucleotides in length. In some embodiments, the GID sequence is less than about 20 nucleotides in length, such as less than about any of 15, 10, 5, or fewer, nucleotides in length.

The methods provided herein may comprise use of a probe that is a 5′ probe or a 3′ probe. In some embodiments, the 5′ probe or the 3′ probe comprises a second target hybridization sequence complementary to a second portion of the mRNA transcript of the plurality of mRNA transcripts. In some embodiments, the 5′ probe or the 3′ probe is between about 5 and 40 nucleotides in length, such as between about any of 5 and 15, 10 and 25, 15 and 30, 20 and 35, and 25 and 40 nucleotides in length. In some embodiments, the 5′ probe or the 3′ probe is greater than about 5 nucleotides in length, such as greater than about any of 10, 15, 20, 25, 30, 35, 40, or more, nucleotides in length. In some embodiments, the 5′ probe or the 3′ probe is less than about 40 nucleotides in length, such as less than about any of 35, 30, 25, 20, 15, 10, 5, or fewer, nucleotides in length. In some embodiments, the second target hybridization sequence of the 5′ probe or the 3′ probe between about 5 and 40 nucleotides in length, such as between about any of 5 and 15, 10 and 25, 15 and 30, 20 and 35, and 25 and 40 nucleotides in length. In some embodiments, the second target hybridization sequence of the 5′ probe or the 3′ probe is greater than about 5 nucleotides in length, such as greater than about any of 10, 15, 20, 25, 30, 35, 40, or more, nucleotides in length. In some embodiments, the second target hybridization sequence of the 5′ probe or the 3′ probe is less than about 40 nucleotides in length, such as less than about any of 35, 30, 25, 20, 15, 10, 5, or fewer, nucleotides in length.

The 3′ loop probe and the 5′ probe, or the 5′ loop probe and the 3′ probe, may be oligonucleotide probes (e.g., DNA molecules). In some embodiments, the probes are RNA molecules or comprise ribonucleotides. In some embodiments, the probe comprises a target hybridization sequence (e.g., a first target hybridization sequence or a second target hybridization sequence) that is a DNA binding region. In some embodiments, the probe comprises a target hybridization sequence that is an RNA binding region. In some embodiments, the probes are modified nucleic acid molecules or contain modified nucleotides or modified nucleosides, such as methylated nucleotides and nucleotide analogs, uracyl, other sugars, and linking groups such as fluororibose and thioate, and nucleotide branches. In some embodiments, the probes may comprise non-nucleotide components. In some embodiments, the probes comprise N3′-P5′ (NP) phosphoramidate, morpholino phosphorociamidate (MF), locked nucleic acid (LNA), 2′-O-methoxyethyl (MOE), or 2′-fluoro, arabino-nucleic acid (FANA).

The 5′ probe or 3′ probe is capable of specifically hybridizing with a portion of the 3′ loop probe or 5′ loop probe, respectively. In some embodiments, the 5′ probe or 3′ probe specifically hybridizes with a first sequence and a second sequence of the 3′ loop probe or the 5′ loop probe, respectively. For example, as shown in FIG. 3 at 308, the 5′ probe comprises a portion that is complementary to the mRNA target sequence, a portion that is complementary to a first sequence at an end of the 3′ loop probe, and a second portion that is complementary to a second sequence at the other end of the 3′ loop probe. Alternatively, the 3′ probe may comprise a portion that is complementary to the mRNA target sequence, a portion that is complementary to a first sequence at an end of the 5′ loop probe, and a second portion that is complementary to a second sequence at the other end of the 5′ loop probe. By specifically hybridizing to both ends of the 3′ loop probe or the 5′ loop probe, the ends of the 3′ loop probe or the 5′ loop probe are brought in proximity to each other, facilitating connecting of the ends of the 3′ loop probe or the 5′ loop probe. In some embodiments, the first sequence and the second sequence of the 3′ loop probe or the 5′ loop probe are each complementary to a portion of the 5′ probe or the 3′ probe, respectively. In some embodiments, the first sequence and the second sequence do not overlap. In some embodiments, the first sequence and the second sequence are located at or near the 3′ terminus and the 5′ terminus of the 3′ loop probe or the 5′ loop probe. In some embodiments, the GID sequence is located between the first sequence and the second sequence of the 3′ loop probe or the 5′ loop probe. In some embodiments, the first sequence and the second sequence are each between about 5 and 40 nucleotides in length, such as between about any of 5 and 15, 10 and 25, 15 and 30, 20 and 35, and 25 and 40 nucleotides in length. In some embodiments, the first sequence and the second sequence are each greater than about 5 nucleotides in length, such as greater than about any of 10, 15, 20, 25, 30, 35, 40, or more, nucleotides in length. In some embodiments, the first sequence and the second sequence are each less than about 40 nucleotides in length, such as less than about any of 35, 30, 25, 20, 15, 10, 5, or fewer, nucleotides in length. In some embodiments, the first sequence and the second sequence are different. In some embodiments, the first sequence and the second sequence are the same.

In some embodiments, the 5′ probe or the 3′ probe hybridizes with the first sequence and the second sequence of the 3′ loop probe or the 5′ loop probe, respectively, via a third and fourth sequence on the 5′ probe or the 3′ probe, respectively. In some embodiments, the first sequence of the 3′ loop probe or the 5′ loop probe hybridizes with the third sequence of the 5′ probe or 3′ probe. In some embodiments, the second sequence of the 3′ loop probe or the 5′ loop probe hybridizes with the fourth sequence of the 5′ probe or the 3′ probe. In some embodiments, the third and fourth sequence of the 5′ probe or the 3′ probe are immediately adjacent to one another. In some embodiments, the third and fourth sequence are not immediately adjacent to one another and instead are separated by a gap, for instance a gap of 1, 2, 3, 4, or 5 nucleotides.

Upon hybridization of the 5′ probe with a corresponding 3′ loop probe or hybridization of the 3′ probe with a corresponding 5′ loop probe (e.g., a first sequence and a second sequence of a 3′ loop probe or a first sequence and a second sequence of a 5′ loop probe), a loop is formed in the 3′ loop probe or the 5′ loop probe. The formation of the loop via hybridization of the 5′ probe brings the ends (e.g., the 3′ and 5′ termini) of the 3′ loop probe into proximity with one another. Alternatively, the formation of the loop via hybridization of the 3′ probe brings the ends (e.g., the 3′ and 5′ termini) of the 5′ loop probe into proximity with one another. The formation of the loop in the 3′ loop probe or in the 5′ loop probe may allow for connecting the ends of the loop in the 3′ loop probe or the loop in the 5′ loop probe to form a circular probe. Thus, in some embodiments, the method further comprises connecting the ends of the loop in one or more oligonucleotide probes (e.g., connecting the ends of the 3′ loop probe or the ends of the 5′ loop probe) to form a circular probe.

The methods provided herein may involve connecting the 5′ and 3′ ends of a 3′ loop probe or a 5′ loop probe. In some embodiments, the 5′ and 3′ ends of the 3′ loop probe or the 5′ loop probe are ligated in order to form a circular probe. In some aspects, the ligation of the ends of the 3′ loop probe or the 5′ loop probe is mediated by the hybridization of one or more sequences of the 3′ loop probe or the 5′ loop probe (e.g., a first sequence and a second sequence) to a 5′ probe or a 3′ probe (e.g., a third sequence and a fourth sequence, respectively) and hybridization of another sequence of the 3′ loop probe or the 5′ loop probe (e.g., a first target hybridization sequence) to a first portion of an mRNA transcript, wherein this mRNA transcript is also hybridized to the 5′ probe or the 3′ probe via a second target hybridization sequence.

In some embodiments, the ligation is an enzymatic ligation. In some embodiments, the enzymatic ligation involves use of a ligase. An RNA ligase, a DNA ligase, or another variety of ligase can be used for the ligation of the ends of the 3′ loop probe or the 5′ loop probe to form a circular probe. In some embodiments, the ligase is a ligase that has a DNA-splinted DNA ligase activity. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a splintR ligase. In some embodiments, the ligase is a T4 DNA ligase.

In some embodiments, the 5′ and 3′ ends of the 3′ loop probe or the 5′ loop probe may be ligated directly or indirectly. “Direct ligation” means that the ends of the 3′ loop probe or the 5′ loop probe hybridize immediately adjacently to one another to form a substrate for a ligase enzyme resulting in their ligation to each other (intramolecular ligation). Alternatively, “indirect ligations” means that the ends of the 3′ loop probe or the 5′ loop probe hybridize non-adjacently to one another, i.e., separated by one or more intervening nucleotides. In some embodiments, the ligation of the 5′ and 3′ ends of the 3′ loop probe of the 5′ loop probe does not require gap filling (e.g., between one or more intervening nucleotides). In other embodiments, the ligation of the 5′ and 3′ ends of the 3′ loop probe or the 5′ loop probe is preceded by gap filling. In some embodiments, gap filling comprises extending the 3′ end of the 3′ loop probe or extending the 5′ end of the 5′ loop probe to fill the gap corresponding to intervening nucleotides.

In some embodiments, the method further comprises amplifying the circular probe formed upon connection (e.g., ligation) of the ends of the 3′ loop probe or the 5′ loop probe, thereby generating an DNA amplicon. In some embodiments, the amplifying comprises performing rolling circle amplification (RCA). In some embodiments, amplification is performed using the circular probe as template and the 5′ probe or the 3′ probe as a primer. In some embodiments, a removing step is performed to remove molecules that are not specifically hybridized to the mRNA transcript and/or the circular probe. Unhybridized molecules may comprise, for example, unhybridized 3′ loop probes and/or unhybridized 5′ probes or unhybridized 5′ loop probes and/or unhybridized 3′ probes. In some embodiments, the removing may comprise a wash, e.g., a stringency wash, to remove unhybridized molecules.

The amplification of the circular probe may comprise isothermal amplification or non-isothermal amplification. In some embodiments, the amplification is RCA. RCA may comprise a linear RCA, a branched RCA, a dendritic RCA, or any combination thereof. Techniques for RCA are known in the art (See, e.g., Baner et al, Nucleic Acids Research, 26:5073-5078, 1998; Lizardi et al, Nature Genetics 19:226, 1998; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801). In some embodiments, the polymerase is phi29 DNA polymerase.

Modified nucleotides may be added during the amplification reaction to incorporate the modified nucleotides in the amplification product (e.g., the DNA amplicon). In some embodiments, the DNA amplicon comprises a modified nucleotide, such as an amine-modified nucleotide. In some embodiments, the amine-modified nucleotide comprises an acrylic acid N-hydroxysuccinimide moiety modification, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

Amplification of the circular probe may produce an DNA amplicon. In some embodiments, the DNA amplicon comprises multiple copies of a target mRNA sequence. In some embodiments, the DNA amplicon is detected and/or sequenced in situ.

In some embodiments, the DNA amplicon is fixed in situ. Fixing the DNA amplicon prevents diffusion of the amplicon within the cell. In some embodiments, the fixing comprises fixing the DNA amplicon to a surface. In some embodiments, the fixing comprises treatment with bis-N-succinimidyl-(pentaethylene glycol) (bis-MHS-PEG). In some embodiments, the fixing occurs at room temperature, e.g., between about 15° C. and about 30° C. The fixing of the DNA amplicon may be for between about 10 minutes and about 1 hour, such as between any of about 10 minutes to 30 minutes, 20 minutes to 40 minutes, 30 minutes to 50 minutes, or 40 minutes to 1 hour. In some embodiments, the DNA amplicon fixing comprises greater than about 10 minutes, such as greater than any of about 20 minutes, 30 minutes, 40 minutes, 50 minutes, 1 hour, or more. In some embodiments, the DNA amplicon fixing comprises less than about 1 hour, such as less than about any of 50 minutes, 40 minutes, 30 minutes, 20 minutes, 10 minutes, or fewer.

### Detection

In some aspects, the provided methods comprise detecting DNA amplicons during the at least one round of RNA FISH. In some embodiments, the provided methods comprise detecting RNA biomarkers or proteins of interest during at least one round of immunoFISH. In some embodiments, the provided methods comprise detecting one or more RNA foci during at least one round of RNA foci FISH. In some embodiments, the detecting comprises imaging a DNA amplicon (which is typically the product of a reverse transcription of an mRNA transcript followed by amplification to form the DNA amplicon during the at least one round of RNA FISH), for example, via binding of a probe or probe set to the DNA amplicon, or a portion thereof. In some embodiments, at least one probe of probe set comprises a detectable label that may be detected (e.g., via imaging). The terms “label” and “detectable label” refer to a detectable moiety that is associated with (e.g., conjugated to) a molecule to be detected, e.g., a probe associated with an DNA amplicon, either directly or indirectly. In some embodiments, the detectable label may be, but is not limited to, a fluorophore, radioactive isotope, fluorescer, chemiluminescer, enzyme, enzyme substrate, enzyme cofactor, enzyme inhibitor, chromophore, dye, metal ions metal sol, ligand (e.g., biotin or haptens), or any combination thereof.

Examples of detectable labels include but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers include, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and the attachment of nucleotides thereto are well known in the art. Exemplary fluorescent labels include those described in, for example, Hoagland, Handbook of Fluorescent Probes and Research Chemicals, Ninth Edition (Molecular Probes, Inc., Eugene, 2002); Keller and Manak, DNA Probes, 2^{nd} Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). Techniques applicable to the provided embodiments include those described in, for example, U.S. Pat. Nos. 4,757,141, 5,151,507 and 5,091,519. In some embodiments, one or more fluorescent dyes are used as labels for labeled target sequences, for example, those described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labelling may also be carried out with quantum dots, such as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264. As used herein, the term “fluorescent label” comprises a signaling moiety that conveys information through the fluorescent absorption and/or emission properties of one or more molecules. Exemplary fluorescent properties comprise fluorescence intensity, fluorescence lifetime, emission spectrum characteristics, and energy transfer.

Examples of commercially available fluorescent nucleotide analogues that may be incorporated into oligonucleotide sequences 77 include, but are not limited to, Cy3-dCTP, Cy3-dUTP, Cy5-dCTP, Cy5-dUTP (Amersham Biosciences, Piscataway, N.J.), fluorescein-!2-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods for custom synthesis of nucleotides having other fluorophores are also known in the art (See, Henegariu et al. (2000) Nature Biotechnol. 18:345).

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some embodiments, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on an oligonucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. As used herein, the term antibody refers to an antibody molecule of any class, or any sub-fragment thereof, such as an Fab.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, an oligonucleotide sequence can be indirectly labeled, especially with a hapten that is then bound by a capture agent, e.g., as described in U.S. Pat. Nos. 5,344,757, 5,702,888, 5,354,657, 5,198,537 and 4,849,336, and PCT publication WO 91/17160. Many different hapten-capture agent pairs are available for use. Exemplary haptens include, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some embodiments, the detecting of the DNA amplicon involves using detection methods such as flow cytometry, sequencing, probe binding and electrochemical detection, pH alteration, catalysis induced by enzymes bound to DNA tags, and/or scanning electron microscopy. In some embodiments, the detecting comprises performing microscopy, scanning mass spectrometry or other imaging techniques described herein. In some embodiments, the detecting comprises determining and/or quantifying a signal, e.g., a fluorescent signal.

In some embodiments, the imaging is carried out using any of various types of microscopy, such as but not limited to, fluorescence microscopy, confocal microscopy, two-photon microscopy, light-field microscopy, intact tissue expansion microscopy, and/or CLARITY™-optimized light sheet microscopy (COLM).

Fluorescence microscopy may be used for detection and imaging. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

Confocal microscopy uses point illumination and a pinhole in an optically conjugate plane in front of the detector to eliminate out-of-focus signals. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution may be at the cost of decreased signal intensity—so long exposures are often required. As only one point in the sample is illuminated at a time, 2D or 3D imaging requires scanning over a regular raster (i.e., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that may be applicable to the present invention include, but are not limited to, bright field microscopy, confocal microscopy, CLARITY™-optimized light sheet microscopy (COLM), oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECS™), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, the detection of the DNA amplicon, and associated mRNA transcript (e.g., mRNA target sequence on the mRNA transcript), occurs during an RNA FISH imaging cycle. Multiple RNA FISH imaging cycles may be performed in order to detect (e.g., image) each of the mRNA transcripts of interest. In each FISH imaging cycle, the plurality of cells comprising DNA amplicons may be contacted with adapter oligonucleotides and detection probes. In some embodiments, the adapter oligonucleotides and detection probes are designed such that each DNA amplicon to be imaged has a single GID sequence and single adapter oligonucleotide/detection probe pair that binds with said GID sequence. Thus enabling multiple colors (e.g., mRNA transcripts) to be imaged simultaneously.

The adapter oligonucleotide is capable of specifically hybridizing with the DNA amplicon. In some embodiments, the adapter oligonucleotide, or a portion thereof, specifically hybridizes with the DNA amplicon, or a portion thereof. In some embodiments, the adapter oligonucleotide specifically hybridizes with a single GID sequence, or a portion thereof, within the DNA amplicon. The adapter oligonucleotide is additionally capable of specifically hybridizing with a detection probe. In some embodiments, the adapter oligonucleotide, or a portion thereof, specifically hybridizes with the detection probe, or a portion thereof. Therefore, the adapter oligonucleotide has dual hybridizing capability with the DNA amplicon and the detection probe, and may serve as an “adapter” between the DNA amplicon and the detection probe. In some embodiments, the adapter oligonucleotide comprises a sequence (e.g., a toehold sequence) that does not hybridize with either the DNA amplicon or the detection probe. In some embodiments, the toehold sequence is between the sequence that specifically hybridizes with the DNA amplicon and the sequence that specifically hybridizes with the detection probe. In some embodiments, the adapter oligonucleotide is between about 5 and 40 nucleotides in length, such as between about any of 5 and 15, 10 and 25, 15 and 30, 20 and 35, and 25 and 40 nucleotides in length. In some embodiments, the adapter oligonucleotide is greater than about 5 nucleotides in length, such as greater than about any of 10, 15, 20, 25, 30, 35, 40, or more, nucleotides in length. In some embodiments, the adapter oligonucleotide is less than about 40 nucleotides in length, such as less than about any of 35, 30, 25, 20, 15, 10, 5, or fewer, nucleotides in length.

The detection probe is capable of specifically hybridizing with the adapter oligonucleotide. In some embodiments, the detection probe, or a portion thereof, specifically hybridizes with the adapter probe, or a portion thereof. The detection probe comprises a detectable label, such as any of the detectable labels described above. In some embodiments, the detection probe comprises a fluorophore. In some embodiments, each detection probe of a plurality of detection probes comprises a unique fluorophore. In some embodiments, various groups of detection probe of a plurality of detection probes each comprises a unique fluorophore, such that there are different groups of detection probes and each individual group comprises a fluorophore that is unique compared to the fluorophores of the other group(s). In some embodiments, the detection probe is between about 5 and 40 nucleotides in length, such as between about any of 5 and 15, 10 and 25, 15 and 30, 20 and 35, and 25 and 40 nucleotides in length. In some embodiments, the detection probe is greater than about 5 nucleotides in length, such as greater than about any of 10, 15, 20, 25, 30, 35, 40, or more, nucleotides in length. In some embodiments, the detection probe is less than about 40 nucleotides in length, such as less than about any of 35, 30, 25, 20, 15, 10, 5, or fewer, nucleotides in length.

In some embodiments, the mRNA transcript is contacted with the adapter oligonucleotides and the detection probes simultaneously. In some embodiments, the mRNA transcript is contacted with the adapter oligonucleotides and the detection probes sequentially, e.g., the mRNA transcript is contacted with the adapter oligonucleotides prior to contacting with the detection probes, or vice versa. In some embodiments, an adapter oligonucleotide hybridizes with the DNA amplicon prior to hybridizing with a detection probe. In some embodiments, an adapter oligonucleotide hybridizes with a detection probe prior to hybridizing with the DNA amplicon. In some embodiments, the detection probes are pre-hybridized with the adapter oligonucleotides prior to contacting the DNA amplicon.

The detection probe hybridized, via an adapter oligonucleotide, with the DNA amplicon may be detected (e.g., imaged) with a microscope (e.g., a confocal microscope). In some embodiments, multiple DNA amplicons (e.g., genes of interest) are imaged in a single imaging cycle. For example, if four unique adapter oligonucleotide/detection probe pairs are used, each imaging cycle detects four genes of interest. Following imaging, toehold displacement oligonucleotides may be used to release the hybridized adapter oligonucleotide/detection probe pair from the DNA amplicon (e.g., the GID sequence of the DNA amplicon). Each toehold displacement oligonucleotide functions to mediate strand displacement of the adapter oligonucleotide/detection probe pair from the DNA amplicon. In some embodiments, the toehold displacement oligonucleotide specifically hybridizes with the toehold sequence and the DNA amplicon hybridization sequence of the adapter oligonucleotide. In this manner, the toehold displacement oligonucleotide first hybridizes with the toehold sequence and initiates the displacement of the adapter oligonucleotide from the DNA amplicon. In some embodiments, the toehold displacement oligonucleotide is between about 5 and 40 nucleotides in length, such as between about any of 5 and 15, 10 and 25, 15 and 30, 20 and 35, and 25 and 40 nucleotides in length. In some embodiments, the toehold displacement oligonucleotide is greater than about 5 nucleotides in length, such as greater than about any of 10, 15, 20, 25, 30, 35, 40, or more, nucleotides in length. In some embodiments, the toehold displacement oligonucleotide is less than about 40 nucleotides in length, such as less than about any of 35, 30, 25, 20, 15, 10, 5, or fewer, nucleotides in length.

In some embodiments, the analysis and/or sequence determination involves washing to remove unbound oligonucleotides (e.g., displaced adapter oligonucleotides, detection probes, and toehold displacement oligonucleotides), thereafter revealing a fluorescent product for imaging. Additional rounds of imaging may be performed to image each gene of interest once the adapter oligonucleotide/detection probe pair is displaced from the DNA amplicon.

### Generation of Proteomic and Morphological Data

Optionally, the method may comprise analyzing the phenotype of a cell or a plurality of cells. For example, analyzing the phenotype of the cell may comprise generating proteomic and/or morphological data for the cell. In some embodiments, analyzing the phenotype of the cell or plurality of cells comprises an assay selected from the group comprising RNA foci/RNA aggregation imaging, antibody-DNA conjugate imaging, label-free imaging, high content imaging, calcium imaging, immunohistochemistry, cell morphology imaging, protein aggregation imaging, cell-cell interaction imaging, live cell imaging, and any other imaging-based assay modality. The cells can additionally be imaged using cellular dyes, bright-field imaging, quantitative phase contrast imaging, or antibody staining to derive morphological phenotypes. In some embodiments, analyzing the phenotype comprises an assay selected from the group comprising RNA foci/RNA aggregation imaging, antibody-DNA conjugate imaging, label-free imaging, high content imaging, calcium imaging, voltage imaging, immunohistochemistry, cell morphology imaging, protein aggregation imaging, cell-cell interaction imaging, live cell imaging, neurite outgrowth assays, phagocytosis assays, immune cell migration assays, immune cell invasion assays, various reporter assays, or a combination thereof.

High content imaging may be used to analyze the phenotype of a cell or a plurality of cells. High content imaging applies automated microscopy, fluorescent detection, and cell-level quantification/advanced statistic algorithms to visualize and quantify complex biological in cell populations. The techniques allow for analysis of how a target (e.g., a cell) interacts with a therapeutic and/or a genetic perturbation. In some embodiments, the method comprises high content imaging of the cell or the plurality of cells.

Calcium imaging may be used to analyze the phenotype of a cell or a plurality of cells. Calcium imaging optically measures the calcium (Ca²⁺) status of a cell or a plurality of cells using calcium indicators. Calcium indicators (e.g., chemical indicators and genetically encoded calcium indicators (GECI)) are fluorescent molecules that respond to the binding of Ca²⁺ ions by fluorescence. Calcium imaging can be used to monitor the electrical activity of neurons in cell culture. In some embodiments, the method comprises calcium imaging of the cell or the plurality of cells.

Voltage imaging may be used to analyze the phenotype of a cell or a plurality of cells. Voltage imaging visualizes a direct indicator of cellular activity by measuring changes in voltage, similar to electrophysiology. Voltage imaging can be used to monitor the electrical activity of neurons in cell culture. In some embodiments, the method comprises voltage imaging of the cell or the plurality of cells.

Immunohistochemistry may be used to analyze the phenotype of a cell or a plurality of cells. This technique involves immunostaining a cell or a plurality of cells with antibodies that specifically bind to an antigen (e.g., protein) of interest in those cells. Therefore, immunohistochemistry allows for the visualization of an antibody-antigen interaction in a cell to identify and quantify the antigen. Immunohistochemistry may comprise chromogenic immunohistochemistry (e.g., an antibody is conjugated to an enzyme that can catalyze a color-producing reaction) and/or immunofluorescence (e.g., an antibody is conjugated to a fluorophore). In some embodiments, immunohistochemistry comprises antibody staining. In some embodiments, the method comprises immunohistochemistry of the cell or the plurality of cells.

Cell morphology imaging may be used to analyze the phenotype of a cell or a plurality of cells. Cell morphology imaging comprises high-resolution imaging to visualize cellular morphology, and may include, but is not limited to, bright-field imaging, quantitative phase contrast imaging, fluorescence imaging, three-dimensional (3D) surface topography imaging, and X-ray imaging. Bright-field light microscopy uses white light to illuminate a sample, and contrast in the sample is caused by attenuation of the transmitted light in dense areas of the sample. Quantitative phase contrast imaging involves quantifying the phase shift that occurs when light waves pass through an optically dense sample. Fluorescence imaging captures images of fluorescent dyes and fluorescent proteins to determine cellular structures and substructures. 3D surface topography imaging comprises imaging techniques that provide quantitative analysis of surface features of cells in 3D space. Finally, X-ray microscopy uses electromagnetic radiation to produce magnified images of objects. In some embodiments, the method comprises cell morphology imaging. In some embodiments, the method comprises bright-field imaging. In some embodiments, the method comprises quantitative phase contrast imaging. In some embodiments, the method comprises fluorescence imaging. In some embodiments, the method comprises 3D surface topography imaging. In some embodiments, the method comprises X-ray imaging.

Protein aggregation imaging may be used to analyze the phenotype of a cell or a plurality of cells. This technique comprises the study of in situ protein aggregation using advanced imaging techniques, in order to capture protein aggregate structures (e.g., motion and distribution of aggregates, and structural changes of aggregates) and aggregate assembly dynamics (e.g., aggregate seeding and expansion). In some embodiments, the method comprises protein aggregation imaging.

Cell-cell interaction imaging may be used to analyze the phenotype of a cell or a plurality of cells. Cell-cell interaction imaging comprises any suitable imaging technique that is capable of capturing the interactions and spatial distributions between cells. In some embodiments, the method comprises cell-cell interaction imaging.

Live cell imaging may be used to analyze the phenotype of a cell or a plurality of cells. Live cell imaging is the visualization of living cells using time-lapse microscopy, such as using bright-field and/or fluorescence live cell imaging. In some embodiments, the method comprises live cell imaging.

Neurite outgrowth assays may be used to analyze the phenotype of a cell or a plurality of cells. Neurite outgrowth assays comprise an in vitro method to evaluate the potential effects of exogenous compounds (e.g., therapeutic compounds) on neuronal activity and development. These assays may be used to determine whether a compound either enhances or inhibits neuritogenesis. In some embodiments, the method comprises performing neurite outgrowth assays.

Phagocytosis assays may be used to analyze the phenotype of a cell or a plurality of cells. Phagocytosis assays measure the engulfment of a substrate in vitro, such as a target cell, by phagocytic cells. Phagocytic cells are a type of cell that has the ability to ingest, and sometimes digest, foreign particles and may include, but are not limited to, monocytes and macrophages, granulocytes, and dendritic cells. In some embodiments, the method comprises performing phagocytosis assays.

Immune cell migration and invasion assays may be used to analyze the phenotype of a cell or a plurality of cells. Immune cell migration and invasion assays can involve quantitating the degree to which invasive cells penetrate a barrier. These assays evaluate the motility and invasiveness of a cell toward a chemo-attractant gradient. In some embodiments, the method comprises performing immune cell migration assays and/or immune cell invasion assays.

Various reporter assays may be used to analyze the phenotype of a cell or a plurality of cells. Reporter assays are typically used to measure the regulatory ability of an unknown DNA-sequence by linking the unknown promoter sequence to a detectable reporter gene, such as luciferase, green fluorescent protein (GFP), etc., whose product can be detected and quantifiably measured. In some embodiments, the method comprises performing reporter assays.

Cellular dyes may be used to analyze the phenotype of a cell or a plurality of cells. Cellular dyes can stain cells in order to enhance visualization of the cell or specific cellular substructures during images. Antibodies (e.g., immunostaining) may be used in combination with cellular dyes. In some embodiments, the method comprises the staining of cells with cellular dyes. In some embodiments, the cells may be stained with CellPaint (e.g., a combination of cellular dyes and antibodies). For example, if the cells are iStels, the cells may be stained with StellatePaint. In some embodiments, the cellular dye and antibody stains are used to segment individual cells (e.g., define the boundaries of individual cells).

### Pooled Optical Screening in Human Cells (POSH) of Genetically Barcoded Cells

Pooled screening approaches enable the study of the impact of background genetics on cell behavior, phenotypes, and disease states. Pooled optical screening (POSH) of genetic perturbations may comprise barcoding cells within a population to correlate a particular perturbation with a phenotype. Each perturbation may be associated with a barcode sequence that can be identified via in situ sequencing. Pooling genetic perturbations offers several advantages, making it practical to conduct comprehensive, genome-scale screens to find relevant components in a biological process. Pooled libraries can be constructed, introduced into cells, and read out as single samples, dramatically reducing the cost, effort, and time needed to perform large-scale screens. Moreover, combining all perturbations into a single sample reduces batch effects across perturbed and control cells, improving statistical power because all cells experience the same conditions.

The methods provided herein involve POSH of a plurality of genetically barcoded cells. In some embodiments, the methods implement pooled optical barcoding and CRISPR-screening strategies. In some embodiments, unique padlock-flanked or otherwise sequenceable barcodes are integrated into a plurality of cells containing a variant of Cas9. In some embodiments, guide RNAs (gRNAs) are identified via in situ sequencing. Exemplary methods of pooled optical screening are known in the art. See, e.g., Feldman et al. (2019) Cell 179(3):787-799.e17, Lawson et al. (2021) Nat Methods 18(4):358-365, and Funk et al. (2021) bioRxiv DOI: 10.1101/2021.11.28.470116. In some embodiments, the gRNAs are directly sequenced. In some embodiments, the gRNAs are indirectly sequenced.

In some embodiments, performing in situ sequencing comprises using fluorescent in situ RNA sequencing (FISSEQ) (Lee et al., Nature Protocols 2015, 10(3):442-58). In this method, a barcode sequence is reverse transcribed in situ using aminoallyl dUTP and adapter sequence-tagged random hexamers. The resulting reverse transcribed barcodes (e.g., cDNA fragments) are fixed to the cellular protein matrix and circularized to form a circular probe. The circular probes are amplified by rolling circle amplification (RCA) followed by sequencing (e.g., sequencing-by-synthesis) and imaging. This method allows for simultaneous detection of tissue-specific gene expression, RNA splicing, post-transcriptional modifications, and preservation of their spatial information. It is a relatively unbiased method and can achieve transcriptome-wide sampling.

In some embodiments, performing in situ sequencing comprises using a padlock in situ sequencing method (Ke et al. Nature Methods 2013, 10(9) 857-60). In this method, a barcode sequence is reverse transcribed into a reverse transcribed barcode (e.g., cDNA). A padlock probe (e.g., at least one padlock probe) then binds to the reverse transcribed barcode with a gap between the probe ends over the bases targeted for sequencing. The gap is filled by DNA polymerization and ligated to form a circular probe. The circular probes are amplified by RCA followed by sequencing (e.g., sequencing-by-synthesis) and imaging. Similar to FISSEQ, padlock in situ sequencing allows for preservation of spatial information of analyzed RNA sequences.

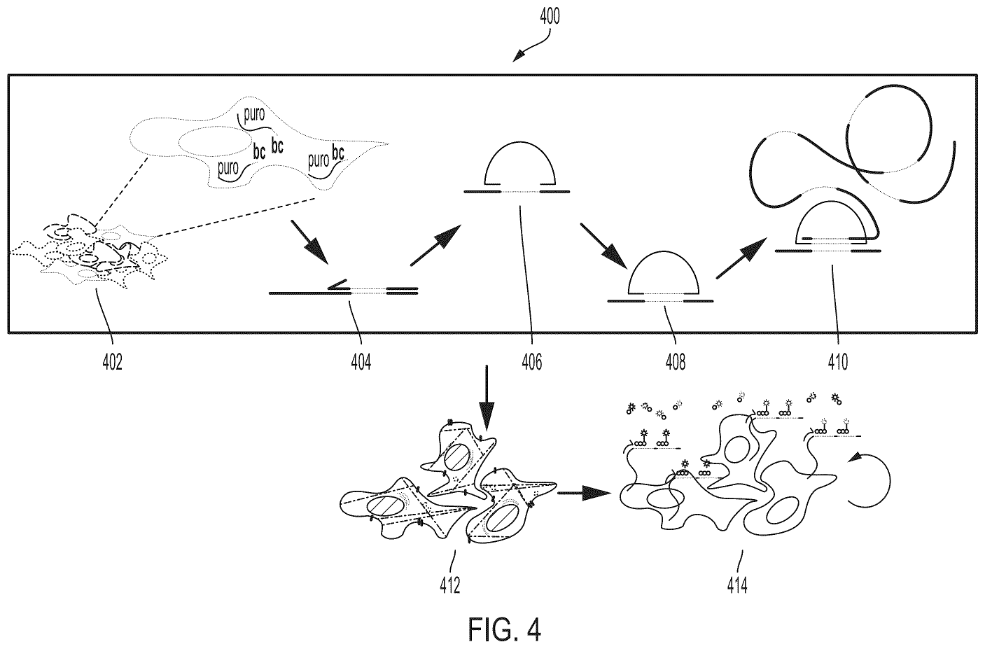

As illustrated in the exemplary process 400 of FIG. 4, a plurality of cells comprising at least one genetic perturbation are fixed at their location, such as by bis-MHS-PEG exposure at 402. At least one cell comprising the at least one genetic perturbation comprises a barcode sequence associated with and identifying the genetic perturbation. The cells are contacted with a reverse transcription primer and an additional fixing, such as by paraformaldehyde (PFA) treatment and/or glutaraldehyde treatment. Cells then undergo reverse transcription, followed by a final PFA fix, generating a reverse transcribed barcode sequence 404. A padlock probe (e.g., at least one padlock probe) is then hybridized to the reverse transcribed barcode sequence 406. The ends of the padlock probe are connected (such as by gap filling) to form a circular probe 408, and a barcode amplicon is formed using the circular probe as a template 410. The barcode amplicon comprises a plurality of copies of the barcode sequence. Optionally, the cells may be stained with a combination of cellular dyes and antibodies to label the phenotypic differences (e.g., StellatePaint imaging), and to segment individual cells to determine the boundaries of each cell of the plurality of cells 412. The barcode amplicon is sequenced (e.g., via in situ sequencing-by-synthesis) in a cyclic manner 414, to identify the genetic perturbation associated with the barcode sequence. Sequencing is continued for multiple cycles, which enables the readout of the bases for each barcode sequence that is expressed in the plurality of cells. In some embodiments, the barcode is ‘oversequenced’, e.g., sequenced for more cycles than needed, allowing for single- or multi-nucleotide corrections to be conducted. Such oversequencing may correct for one or more nucleotides in a barcode that was incorrectly identified. This process accurately predicts which barcode sequence is present in each cell, allowing a readout of which genetic perturbation that cell received.

Following POSH sequencing of the genetic perturbations, each cell of the plurality of cells can be assigned a transcriptome (e.g., based on FISH imaging cycles such as RNA FISH and/or RNA foci FISH), a morphology/proteome (e.g., based on CellPaint imaging and/or immunoFISH), and a perturbation readout (e.g., based on POSH sequencing results).

### Reverse Transcription

The method comprises reverse transcribing a barcode sequence associated with a genetic perturbation in at least one cell of a plurality of cells. A cell may be fixed (e.g., via treatment with bis-MHS-PEG exposure) in its location prior to reverse transcription. Reverse transcription of the barcode sequence allows for the generation of cDNA fragments (e.g., reverse transcribed barcode sequences), which may be subsequently amplified and detected by sequencing.

In some embodiments, the cell is contacted with a reverse transcription primer. In some embodiments, the cell is contacted with a reverse transcription primer simultaneously with fixing the cell. In some embodiments, the cell is fixed via treatment with PFA or methanol. In some embodiments, the reverse transcription primer hybridizes with the barcode sequence, or a portion thereof. In some embodiments, the barcode sequence is reverse transcribed to form a reverse transcribed barcode sequence.

Modified nucleotides (such as amine-modified nucleotides or locked nucleic acids) may be added to the reverse transcription primer (for example, to produce primers with locked nucleic acids at their 5′ end), for incorporation of the modified nucleotides in the reverse transcribed barcode sequence. In some embodiments, the reverse transcribed barcode sequence comprises a modified nucleotide, such as an amine-modified nucleotide. In some embodiments, the amine-modified nucleotide comprises an acrylic acid N-hydroxysuccinimide moiety modification, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

### Probe Hybridization, Probe Connection, and Amplification

The present invention comprises amplifying a barcode sequence associated with a genetic perturbation in at least one cell of a plurality of cells. Amplification may involve hybridization of a padlock probe to a reverse transcribed barcode sequence, connecting the ends of the padlock probe to form a circular probe, and forming a barcode amplicon using the circular probe as a template. The barcode amplicon comprises multiple copies of the barcode sequence, which may be sequenced to identify a genetic perturbation associated with a particular cell.

In some aspects, a padlock probe may be hybridized to the reverse transcribed barcode sequence. In some embodiments, the padlock probe comprises a modification. In some embodiments, the padlock probe is phosphorylated, such as 5′ phosphorylation. In some embodiments, the padlock probe comprises a first barcode hybridization sequence and a second barcode hybridization sequence. In some embodiments, the first barcode hybridization sequence and the second barcode hybridization sequence are not immediately adjacent to each other, e.g., the first and second barcode hybridization sequences are separated by 10 or more intervening nucleotides. In some embodiments, the first barcode hybridization sequence and the second barcode hybridization sequence comprise different sequences. In some embodiments, the first barcode hybridization sequence and the second barcode hybridization sequence comprise the same sequences. In some embodiments, the reverse transcribed barcode sequence comprises a first padlock probe hybridization sequence and a second padlock probe hybridization sequence flanking a target sequence. In some embodiments, the first padlock probe hybridization sequence and the second padlock probe hybridization sequence are not immediately adjacent to each other, e.g., the first and second barcode hybridization sequences are separated by 10 or more intervening nucleotides, such as the target sequence. In some embodiments, the first padlock probe hybridization sequence and the second padlock probe hybridization sequence comprise different sequences. In some embodiments, the first padlock probe hybridization sequence and the second padlock probe hybridization sequence comprise the same sequences. In some embodiments, the first barcode hybridization sequence hybridizes with a first padlock probe hybridization sequence and the second barcode hybridization sequence hybridizes with a second padlock probe hybridization sequence.

Upon hybridization of the padlock probe (e.g., the first barcode hybridization sequence and the second barcode hybridization sequence of the padlock probe) with the reverse transcribed barcode sequence (e.g., the first padlock probe hybridization sequence and the second padlock probe hybridization sequence of the reverse transcribed barcode sequence), the ends (e.g., the 3′ and 5′ termini) of the padlock probe are brought into proximity with one another.

The methods provided herein may involve connecting the 5′ and 3′ ends of a padlock probe. In some embodiments, the 5′ and 3′ ends of the padlock probe are ligated in order to form a circular probe. In some embodiments, the ligation of the 5′ and 3′ ends of the padlock probe is preceded by gap filling. In some embodiments, gap filling comprises extending the 3′ end of the padlock probe to fill the gap corresponding to intervening nucleotides. In some embodiments, a DNA polymerase is used for gap filling. In some embodiments, the DNA polymerase may be TaqIT.

In some embodiments, the 5′ and 3′ ends of the padlock probe may be ligated directly or indirectly. In some embodiments, the ligation is an enzymatic ligation. In some embodiments, the enzymatic ligation involves use of a ligase. An RNA ligase, a DNA ligase, or another variety of ligase can be used for the ligation of the ends of the 3′ loop probe to form a circular probe. In some embodiments, the ligase is a ligase that has a DNA-splinted DNA ligase activity. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is Ampligase.

In some embodiments, the gap filling followed by ligation comprises contacting the cells with TaqIT and Ampligase.

In some embodiments, the method further comprises amplifying the circular probe formed upon connection (e.g., ligation preceded by gap filling) of the ends of the padlock probe, thereby generating a barcode amplicon. In some embodiments, the amplifying comprises performing rolling circle amplification (RCA). In some embodiments, amplification is performed using the circular probe as a template. In some embodiments, a removing step is performed to remove molecules that are not specifically hybridized to the reverse transcribed barcode sequence and/or the circular probe. Unhybridized molecules may comprise, for example, unhybridized padlock probe. In some embodiments, the removing may comprise a wash, such as a stringency wash, to remove unhybridized molecules.

RCA is performed as described herein, generating a barcode amplicon comprising a plurality of copies of the barcode sequence. The amplification of the circular probe may comprise isothermal amplification or non-isothermal amplification. In some embodiments, the amplification is RCA. RCA may comprise a linear RCA, a branched RCA, a dendritic RCA, or any combination thereof. Techniques for RCA are known in the art (See, e.g., Baner et al, Nucleic Acids Research, 26:5073-5078, 1998; Lizardi et al, Nature Genetics 19:226, 1998; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801). In some embodiments, the polymerase is phi29 DNA polymerase.

Amplification of the circular probe may produce a barcode amplicon. In some embodiments, the barcode amplicon comprises multiple copies of the barcode sequence, corresponding to a genetic perturbation. In some embodiments, the barcode amplicon is detected and/or sequenced in situ.

### Sequencing

In some aspects, the provided methods involve analyzing, such as, detecting, one or more sequences present in the barcode amplicon. In some embodiments, the analysis comprises determining the sequence of all or a portion of the barcode amplicon. In some embodiments, the sequence of all or a portion of the barcode amplicon is indicative of the identity of a barcode sequence associated with a genetic perturbation.

In some embodiments, the sequencing involves sequencing by hybridization, sequencing by ligation, sequencing by synthesis, sequencing by binding, and/or fluorescent in situ sequencing (FISSEQ), padlock in situ sequencing, hybridization-based in situ sequencing and/or wherein the in situ hybridization comprises sequential fluorescent in situ hybridization. In some embodiments, the sequencing comprises detecting a polymer generated by a hybridization chain reaction (HCR) reaction, see e.g., US2017/0009278, which is incorporated herein by reference, for exemplary probes and HCR reaction components. In some embodiments, the detection (e.g., sequencing) comprises hybridizing to the barcode amplicon a detection oligonucleotide labeled with a fluorophore, an isotope, a mass tag, or a combination thereof. In some embodiments, the detection comprises imaging the barcode amplicon, wherein the barcode amplicon comprises multiple copies of the barcode sequence associated with a genetic perturbation.

In some embodiments, sequencing of the barcode amplicon can be performed in situ. In situ sequencing typically involves incorporation of a labeled nucleotide (e.g., fluorescently labeled mononucleotides or dinucleotides) in a sequential, template-dependent manner, or hybridization of a labeled primer (e.g., a labeled random hexamer) to a nucleic acid template such that the identities (i.e., nucleotide sequence) of the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343(6177), 1360-1363. In addition, examples of methods and systems for performing in situ sequencing are described in US 2016/0024555, US 2019/0194709, and in U.S. Pat. Nos. 10,138,509, 10,494,662 and 10,179,932.

In some embodiments, sequencing can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, US 2011/0059865, US 2005/0100900, U.S. Pat. No. 9,217,178, US 2009/0118128, US 2012/0270305, US 2013/0260372, and US 2013/0079232.

Additional techniques for in situ sequencing comprise, but are not limited to, STARmap (described for example in Wang et al., (2018) Science, 361(6499) 5691), MERFISH (described for example in Moffitt, (2016) Methods in Enzymology, 572, 1-49), hybridization-based in situ sequencing (HybISS) (described for example in Gyllborg et al., Nucleic Acids Res (2020) 48(19):e112, and FISSEQ (described for example in US 2019/0032121).

In some aspects, the sequencing is carried out at room temperature for best preservation of tissue morphology with low background noise and error reduction. In some embodiments, the sequencing comprises eliminating error accumulation as sequencing proceeds. In some embodiments, the sequencing involves washing to remove unbound oligonucleotides, thereafter revealing a fluorescent product for imaging. In some embodiments, imaging is performed as described herein.

### Computational Analysis

The methods of the invention, in some embodiments, comprise processing images (such as FISH images, segmentation images, and/or POSH images). In order to obtain the images of the same cell with different readouts across multiple rounds of imaging, the images may be processed and/or transformed. Processing and/or transforming allows for the same cell to be identified and aligned, and various phenotypic readouts to be assigned to a particular cell comprising a particular genetic perturbation (as identified by the associated barcode sequence). In some embodiments, the processing and/or transformation involves computational analysis.

Each round of FISH processing followed by imaging produces a set of multi-channel images that represent a state of the cell as measured by a set of markers. At the end of N rounds of FISH, the system obtains a set of at least ‘N’ different sets of images of the same cell samples with different measurements. The cells are further processed and imaged using the in-situ sequencing (POSH) technique to obtain the identities of the cells using a barcoding scheme. Each of the rounds of FISH images and in situ sequencing cycles (f POSH cycles) can be imaged using the same or different microscopes and involves manual/automated transfer and handling of plates that results in each round of images having its own coordinate system that is not directly related to the other rounds of imaging.

In order to obtain the images of the same cell with different readouts across multiple rounds of imaging, the images need to be processed such that the system can associate each cell with a cell identifier, a portion of each FISH image, and the corresponding barcode sequence (e.g., POSH image).

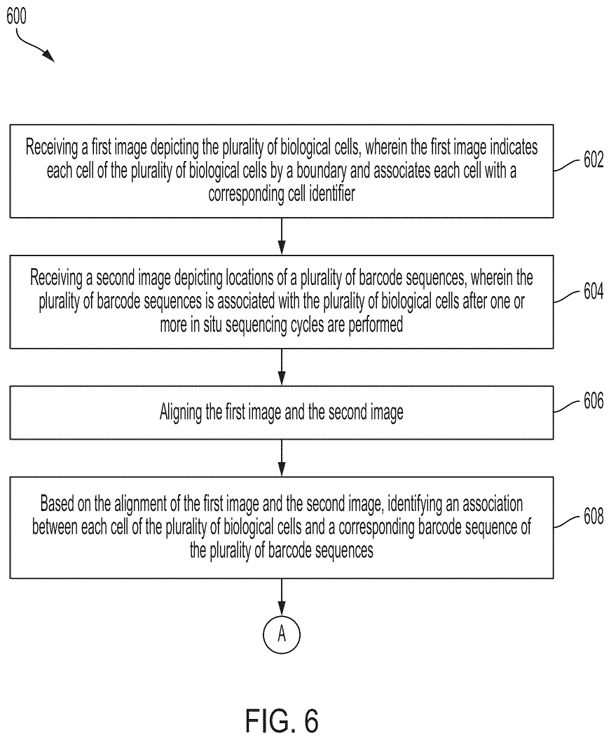

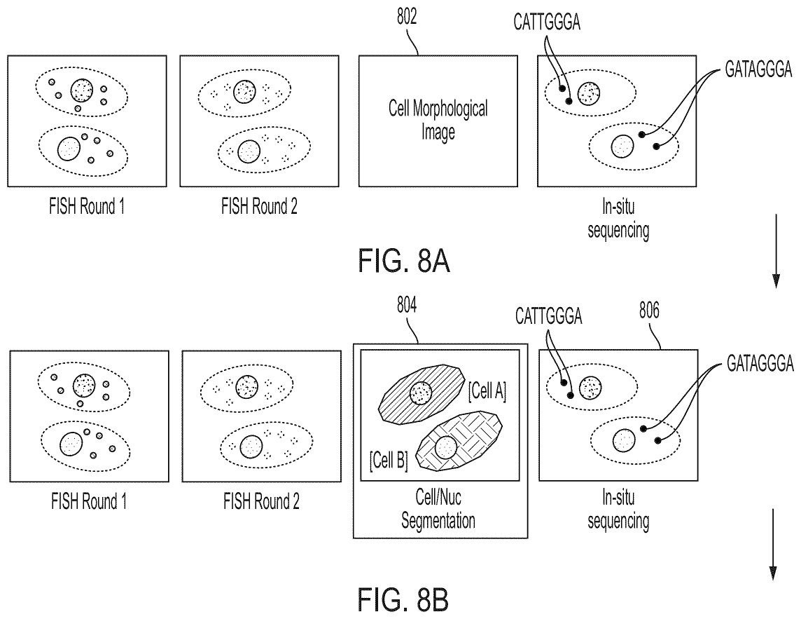

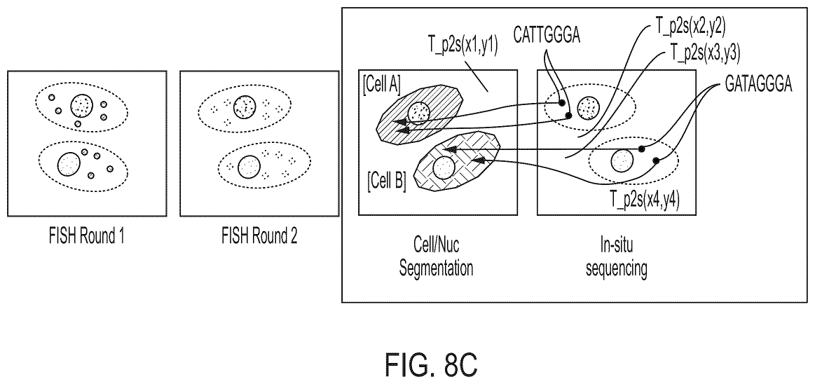

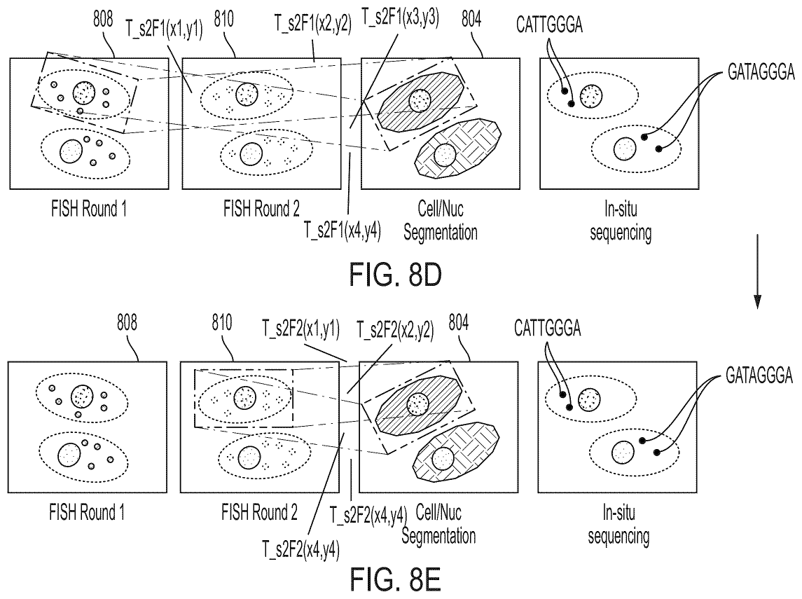

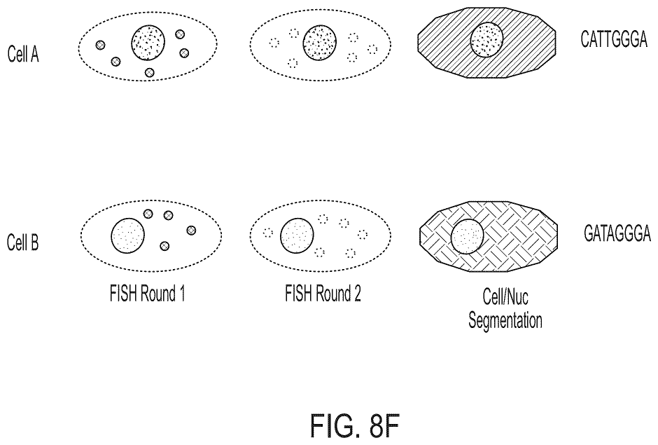

FIG. 6 illustrates an exemplary process 600 for analyzing known phenotypes or identifying new phenotypes of a plurality of cells. Process 600 is at least partially performed, for example, using one or more electronic devices implementing a software platform. In some examples, process 600 is performed using a client-server system, and the blocks of process 600 are divided up in any manner between the server and one or more client devices. In other examples, process 600 is performed using only one or more client devices. In process 600, some blocks are, optionally, combined, the order of some blocks is, optionally, changed, and some blocks are, optionally, omitted. In some examples, additional steps may be performed in combination with the process 600. Accordingly, the operations as illustrated (and described in greater detail below) are exemplary by nature and, as such, should not be viewed as limiting. The process 600 is described below with reference to FIGS. 8A-8F, which illustrate an exemplary process for processing exemplary images to analyze known phenotypes or identify new phenotypes of two cells.

At block 602, an exemplary system (e.g., one or more electronic devices) receives a first image depicting the plurality of cells. The first image indicates each cell of the plurality of cells by a boundary and associates each cell of the plurality of cells with a corresponding cell identifier.

In some embodiments, the first image is generated based on an image depicting morphological characteristics of the plurality of cells. The system can perform segmentation on the morphological image to detect the plurality of cells and identify the boundaries of the plurality of cells in the morphological image. For example, the system can compute cell and nuclei segmentation masks based on the morphological image. Based on the segmentation, the system can generate the first image to represent each cell of the plurality of cells by a boundary and associates each cell of the plurality of cells with a corresponding cell identifier.

For example, in the depicted example in FIGS. 8A-8B, the system obtains an image 802 depicting morphological characteristics of the plurality of cells. The system performs segmentation on the image 802 to detect two cells depicted in the image 802 and identify the spanning regions of the two cells. Accordingly, as shown in FIG. 8B, the system obtains image 804 (i.e., the first image in block 602), which indicates the spanning regions of the two cells and associates the two cells with corresponding cell identifiers or labels, e.g., Cell A and Cell B.

In some embodiments, the morphological image 802 can be a morphology stained image generated using techniques such as CellPaint, StellatePaint, etc. In some embodiments, the morphological image 802 is a phase image such as a quantitative phase contrast (QPC) image depicting the positional and morphological characteristics in particular cellular substructures. In some embodiments, the phase image can be obtained using bright-field imaging and other low resource, non-destructive imaging techniques to recreate high content images with sufficient richness and depth for downstream processing. For example, one or more machine-learning models can be configured to transform images of a first modality (e.g., bright-field images) into images of a second modality (e.g., phase images). Accordingly, phase images can be generated at scale and in a low-cost and non-destructive manner. In some embodiments, the machine-learning model is a generative adversarial network model comprising a discriminator and a generator, and can be trained using ground truth images of the first modality and images of the second modality. Additional information of the image transformation models can be found in U.S. application Ser. No. 17/480,047 titled “BIOLOGICAL IMAGE TRANSFORMATION USING MACHINE-LEARNING MODELS,” which is incorporated herein by reference in its entirety. The imaging stage may generate phase images.

At block 604, the system receives a second image depicting locations of a plurality of barcode sequences. The plurality of barcode sequences are associated with the plurality of cells after one or more in situ sequencing cycles are performed. In some embodiments, the in situ sequencing comprises sequencing a barcode sequence associated with a genetic perturbation. In some embodiments, the in situ sequencing of the barcode sequence comprises sequencing-by-synthesis. In some embodiments, the in situ sequencing generates one or more pooled optical screening (POSH) images. The POSH techniques may be performed according to any of the POSH methods described herein (e.g., in the “Pooled optical screening (POSH) of genetically barcoded cells” section above). The second image depicts locations of a plurality of barcode sequences. The plurality of barcode sequences are associated with the plurality of cells after one or more in situ sequencing cycles are performed. For example, in the depicted example in FIG. 8B, the image 806 depicts locations of two barcode sequences, which are associated with the two cells, respectively.

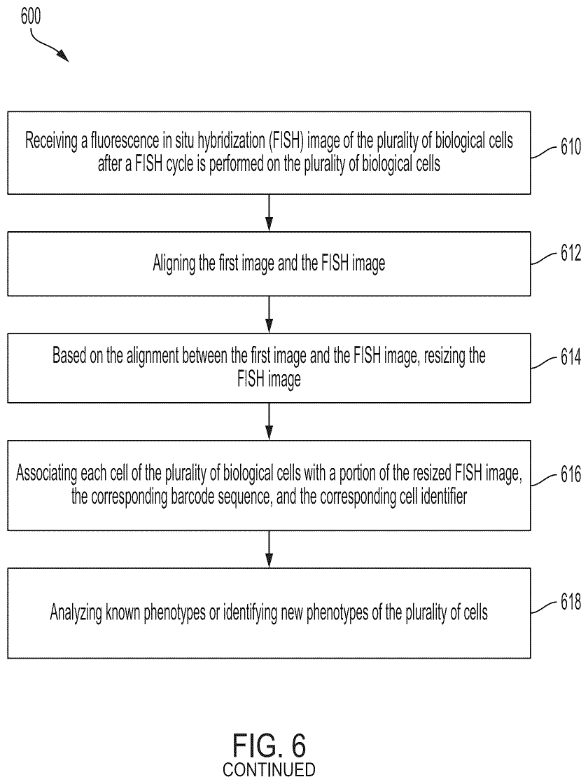

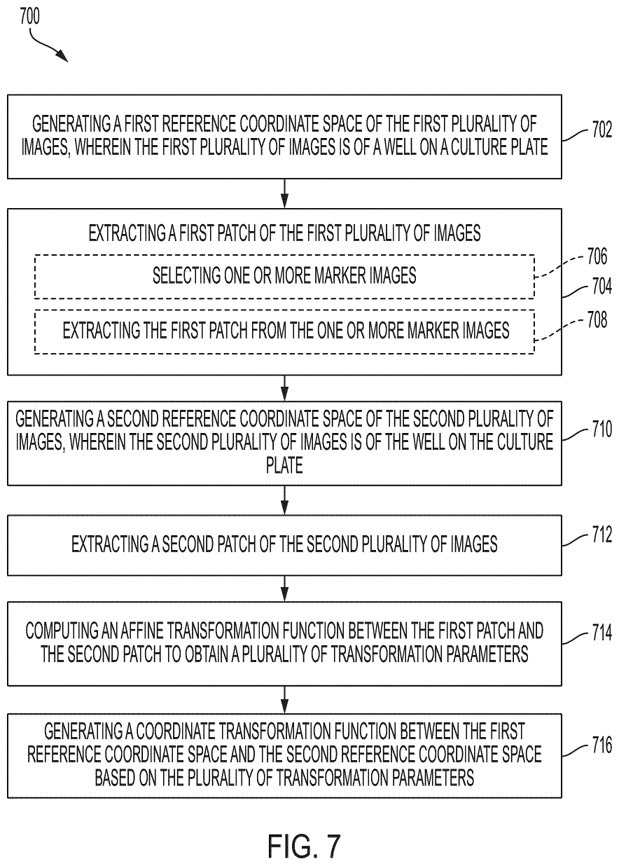

At block 606, the system aligns the first image and the second image. In some embodiments, the system computes a transformation function from the second image to the first image. In some embodiments, the first image comprises a first set of one or more field-of-view images, wherein each field-of-view image depicts a portion of the well that the plurality of cells are placed in, and the second image comprises a second set of one or more field-of-view images, wherein each image depicts a portion of the well that the plurality of cells are placed in. FIG. 7 illustrates an exemplary computer-implemented process 700 for aligning between a first set of one or more images (e.g., from a first image acquisition) and the second set of one or more images (e.g., from a second image acquisition). Additional details can be found in in U.S. Provisional Application No. 63/150,979 titled “SYNTHETIC BARCODING OF CELL LINE BACKGROUND GENETICS,” which is incorporated herein by reference in its entirety.

At block 608, the system, based on the alignment of the first image and the second image, identifies an association between each cell of the plurality of cells and a corresponding barcode sequence of the plurality of barcode sequences. For example, in the depicted example in FIG. 8C, the system applies the first transformation to locations of the barcode sequence “CATTGGGA” to obtain corresponding coordinate locations in the image 804. Because the transformed locations are located within the boundary of Cell A, the system determines that the barcode sequence “CATTGGGA” is associated with Cell A. Similarly, the system applies the first transformation to locations of the barcode sequence “GATAGGGA” to obtain corresponding coordinate locations in the image 804. Because the transformed locations are located within the boundary of Cell B, the system determines that the barcode sequence “GATAGGGA” is associated with Cell B. This step provides the perturbation identity from in-situ sequencing assigned to each cell identifier from cell segmentation.

At block 610, the system receives a fluorescence in situ hybridization (FISH) image of the plurality of cells after a FISH cycle is performed on the plurality of cells. FISH images may be generated according to the methods provided herein. For example, in the depicted example in FIG. 8D, the system receives two FISH images corresponding to two FISH rounds, 808 and 810. In some embodiments, the two FISH images 808 and 810 are captured using different microscopes. In some embodiments, the FISH images and the image 804 are captured using different microscopes. In some embodiments, the first image is obtained based on the one or more FISH images of the plurality of cells.

At block 612, the system aligns the first image and the FISH image. In some embodiments, the alignment comprises computing a transformation function from the first image to the FISH image. For example, in the depicted example in FIG. 8D, the system can compute a transformation function from the image 804 to 808. Further, in the depicted example in FIG. 8E, the system can compute a transformation function from the image 804 to 810.

In some embodiments, the first image comprises a set of one or more field-of-view images each depicting a portion of the well that the plurality of cells are placed in, and the FISH image comprises a set of one or more field-of-view images each depicting a portion of the well that the plurality of cells are placed in. The alignment between the first image and the FISH image can be performed in a similar manner to block 606 described above.

At block 614, the system, based on the alignment between the first image and the FISH image, resizes the FISH image. In some embodiments, the system obtains one or more extremity points of a cell of the plurality of cells in the first image, applies the transformation from the first image to the FISH image to locations of the one or more extremity points to obtain corresponding locations in the FISH image, based on the obtained corresponding locations in the FISH image, obtains a boundary of the cell in the FISH image; and resizes the FISH image such that the cell in the resized FISH image is of the same or substantially similar size as the cell in the first image.

For example, in the depicted example in FIG. 8D, the system obtains four extremity points of Cell A in the image 804, applies the transformation from the image 804 to the FISH image 808 to locations of the four extremity points to obtain corresponding locations in the FISH image 808. Based on the obtained corresponding locations in the FISH image 808, the system obtains a rectangular boundary of the cell in the FISH image 808 and resizes the FISH image 808 such that the cell in the resized FISH image is of the same or substantially similar size as Cell A in the first image.

As another example, in the depicted example in FIG. 8E, the system obtains four extremity points of Cell A in the image 804, applies the transformation from the image 804 to the FISH image 810 to locations of the four extremity points to obtain corresponding locations in the FISH image 810. Based on the obtained corresponding locations in the FISH image 810, the system obtains a rectangular boundary of the cell in the FISH image 810 and resizes the FISH image 810 such that the cell in the resized FISH image is of the same or substantially similar size as Cell A in the first image. Accordingly, both FISH images are resized such that any cell is of a fixed size across the FISH images and the image 804.

At block 616, the system associates each cell of the plurality of cells with a portion of the resized FISH image, the corresponding barcode sequence, and the corresponding cell identifier. In the depicted example in FIG. 8F, the system associates Cell A (i.e., the cell identifier) with: a portion of the resized FISH image 808 depicting Cell A, a portion of the resized FISH image 810 depicting Cell A, the corresponding barcode sequence, a portion of the image 804 depicting Cell A. Similarly, the system associates Cell B (i.e., the cell identifier) with: a portion of the resized FISH image 808 depicting Cell B, a portion of the resized FISH image 810 depicting Cell B, the corresponding barcode sequence, and a portion of the image 804 depicting Cell B.

At block 618, known phenotypes or identifying new phenotypes of the plurality of cells can be analyzed. In some embodiments, analyzing the phenotype of the plurality of cells comprises an assay selected from the group consisting of RNA foci/RNA aggregation imaging, antibody-DNA conjugate imaging, label-free imaging, high content imaging, calcium imaging, immunohistochemistry, cell morphology imaging, protein aggregation imaging, cell-cell interaction imaging, live cell imaging, and any other imaging-based assay modality. In some embodiments, analyzing the phenotype of the plurality of cells comprises an assay selected from the group consisting of RNA foci/RNA aggregation imaging, antibody-DNA conjugate imaging, label-free imaging, high content imaging, calcium imaging, immunohistochemistry, cell morphology imaging, protein aggregation imaging, cell-cell interaction imaging, and live cell imaging. In some embodiments, analyzing the phenotype comprises generating proteomic and/or morphological data for the plurality of cells.

FIG. 7 illustrates an exemplary computer-implemented process 700 for aligning between a first set of one or more images (e.g., from a first image acquisition) and the second set of one or more images (e.g., from a second image acquisition). The first and second acquisitions may be different in terms of coverage of the well, image resolution, image size, and imager type, and the relative position of the well may have been shifted/rotated, but the underlying object(s) being imaged are the same. The process 700 can be performed at least in part using one or more electronic devices. In some embodiments, the blocks of 700 can be divided up between multiple electronic devices. Some blocks can be optionally combined, the order of some blocks can be optionally changed, and some blocks are optionally omitted. In some examples, additional steps may be performed in combination with the process. Accordingly, the operations as illustrated are exemplary by nature and, as such, should not be viewed as limiting.

In the exemplary process 700, the first set of one or more images and the second set of one or more images are both of a well on a culture plate, but they are from two different image acquisitions. While the content in the well remains the same between the two image acquisitions, the imaging settings may have changed between the two image acquisitions. For example, the first set of one or more images may be captured using a first imager and the second set of one or more images may be captured using a second imager. As another example, the first set of one or more images may have a different resolution or magnification than the second set of one or more images. As another example, the first set of one or more images and the second set of one or more images provide different coverage of the well (e.g., the second coverage may be of a different size and/or may be shifted comparing to the first coverage). As another example, the first set of one or more images and the second plurality of images are taken at different times.

At block 702, an exemplary system (e.g., one or more electronic devices) generates a first reference coordinate space of the first set of one or more images. In other words, the system assigns coordinate values of the first reference coordinate space to pixels of at least one of the first set of one or more images. The first set of one or more images can comprise one or more sets of images corresponding to one or more acquisitions. The reference coordinate space can be computed at the well level. Specific locations of the well can be assigned values on the first reference coordinate space. As an example, the center of the well can be assigned as the origin (i.e., 0) of the first reference coordinate space. Further, the left- and right-most points of the well can be assigned the values of −X and X on the x-axis and the top- and bottom-most points of the well can be assigned the values of −Y and Y on the y-axis, where X and Y are predefined numbers. In some embodiments, the values of X and Y are based on physical dimensions of the field of view. In some embodiments, the values of X and Y are obtained from image acquisition metadata, which can be provided by the imager.

In some embodiments, the values of coordinates in pixel space can be X=image_size_x (in pixels) and Y=image_size_y (in pixels). In pixel space, the coordinates are with respect to the image dimensions (in pixels). In physical dimensions (i.e., the first coordinate space), the system constructs a coordinate space that is with respect to the microscope stage. Based on the physical dimensions (e.g., field dimensions in micrometers) of the field from the microscope (e.g., metadata), the system can translate the pixel space to the physical space obtained from microscope.

Each image of the first set of one or more images can be associated with the first reference coordinate space. In some embodiments, the system detects one or more physical characteristics of the well in an image (e.g., edge of the well, shape of the well, location of the well), and associates the image with the first reference coordinate space accordingly. For example, the system can detect the leftmost point and rightmost point of the well in the images, and assign the values of −X and X accordingly. Further, the system can compute the overlap ratio. The overlap ratio can be part of the metadata information from the imager (e.g., the microscope metadata) or can be calculated using redundant image information between the two images. Based on the overlap ratios and the specific locations on the well, the system can assign coordinate values (e.g., X. Y) to pixels of the images.

In some embodiments, the system can compute a global coordinate space with respect to the well position in the microscope stage in physical dimensions (e.g., using metadata of the imager). For example, the position of the center of each field-of-view image can be measured a priori based on the device and acquisition settings, and the overlap ratio and well extrema are computed using the positions of the field-of-view images and the known image dimensions. In some embodiments, the first coordinate space can be artificially simulated using the pixel's physical dimensions and overlap ratios. For example, in cases where the microscope device metadata does not contain the information for positions (i.e. physical coordinate locations) of the field of view images, the coordinate space can be created by identifying the order of field image acquisition in the well, the image sizes, and the overlap ratio.

At 704, the system extracts a first patch of the first set of one or more images. In some embodiments, block 704 includes blocks 706 and 708, as described below.

At block 706, the system selects one or more images that capture one or more landmarks (i.e., marker images) from the first set of one or more images. The landmarks can be information within the wells such as one or more nuclei, one or more cells, one or more beads (obtained from fluorescence markers or segmentation from bright-field or quantitative phase contrast images). The landmarks can also be information of the well such as the boundary of the well and the center of the well. In some embodiments, the selection of marker images can be performed using one or more machine-learning models.

At block 708, the system extracts the first patch from the one or more marker images. The first patch may be one of the marker images, or a portion of one of the marker images. The patch can be extracted such that it captures a particular object or marker or a particular location of the well (e.g., center of the well). The size of the patch is determined such that the patch is large enough to capture the maximum allowable tolerance limit of motion/shift between the acquisitions, as described below with reference to block 712. In some embodiments, the size and location of the patch are determined empirically based on an allowable tolerance threshold. For example, if the system aims to allow up to a shift of 1 mm between the two acquisitions, the system can take a patch size corresponding to x*1 mm in the corresponding image space where x>1. The location of the patch can be anywhere provided we have the patch size corresponding to the tolerance value. In some embodiments, the system extracts the first patch by sparse sampling of the marker images either at random locations in the well or at a fixed location between the two acquisitions. For example, a well can be covered by hundreds of images. Sparse sampling involves choosing a subset of these images corresponding to random physical locations in a well to obtain the first patch. The patch could also be constructed by combining multiple images corresponding to a fixed well location (e.g., the center of the well).

At block 710, the system generates a second reference coordinate space of the second set of one or more images. In other words, the system assigns coordinate values of the second reference coordinate space to pixels of at least one of the second set of one or more images. The second set of one or more images is of the same well on the culture plate. As discussed above, the second set of one or more images can be from a second image acquisition. The first and second acquisitions may be different in terms of coverage of the well, image resolution, image size, and imager type, and the relative position of the well may have been shifted/rotated, but the underlying object(s) being imaged are the same. The generation of the second reference coordinate space at block 710 can be performed in a similar manner as, but independently from, block 702.

At block 712, the system extracts a second patch of the second set of one or more images. The extraction of the second patch can be performed in a similar manner as block 704. For example, one or more marker images can be selected from the second set of one or more images. The second patch can then be extracted from the one or more marker images. The second patch can be extracted such that it captures the same object or marker or the same location of the well (e.g., center of the well) captured in the first patch.

The size of the first and second patches are determined such that the patches are large enough to capture the maximum allowable tolerance limit of motion/shift between the acquisitions. For example, if both the first patch and the second patch capture the center of the well, both patches need to be large enough to capture translation, rotation, and scaling around the center of the well in both the acquisitions.

At 714, the system computes an affine transformation function between the first patch and the second patch to obtain a plurality of transformation parameters. The transformation parameters can comprise one or more of: a translation parameter, a scaling parameter, and a rotation parameter.

At 716, the system generates a coordinate transformation function between the first reference coordinate space and the second reference coordinate space based on the plurality of transformation parameters.

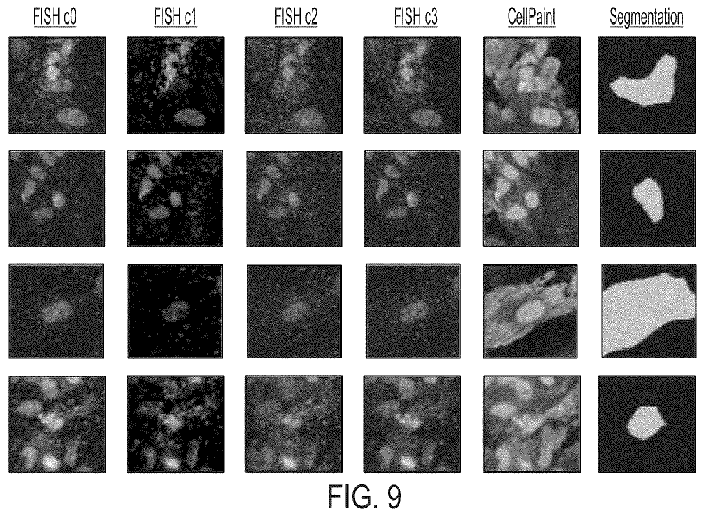

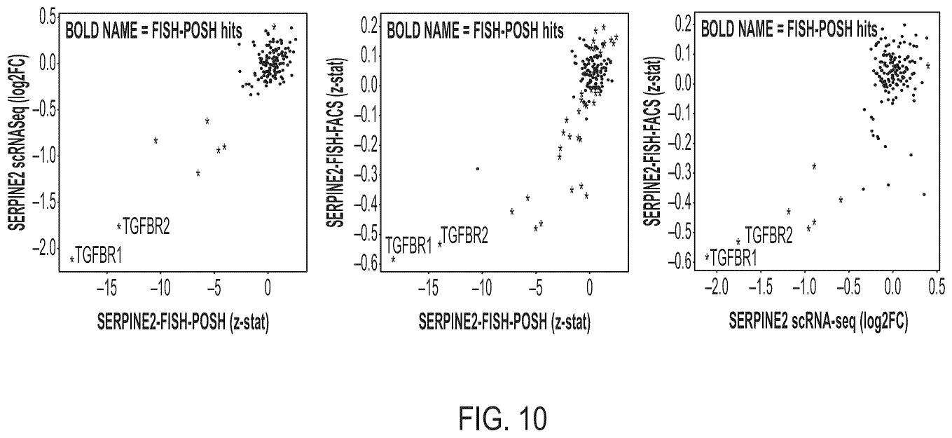

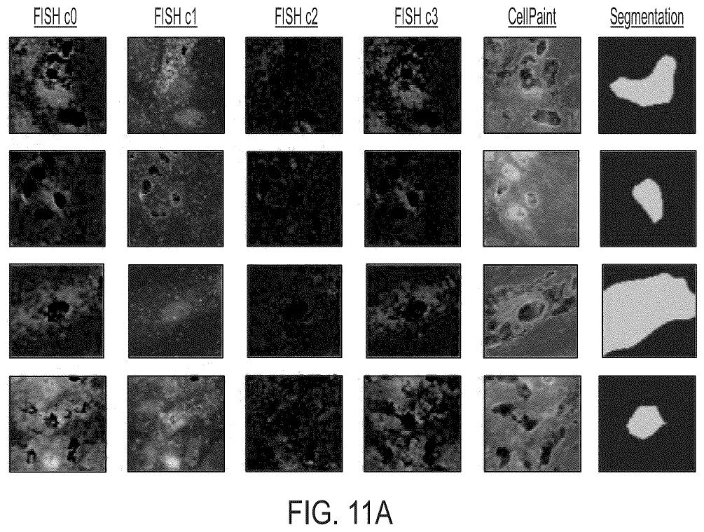

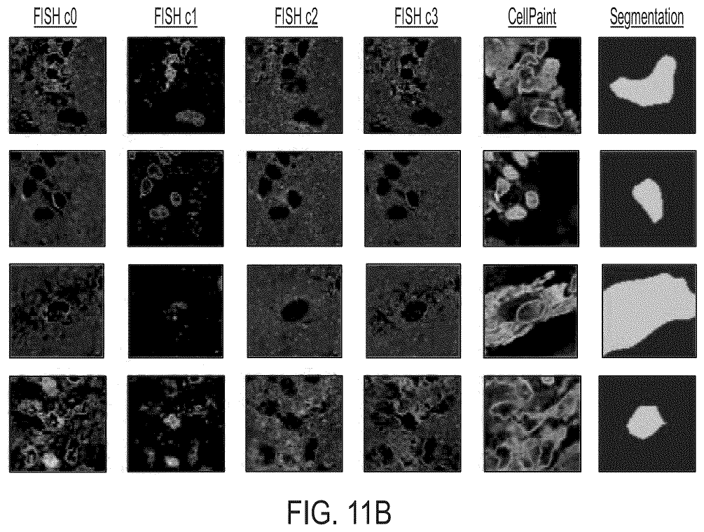

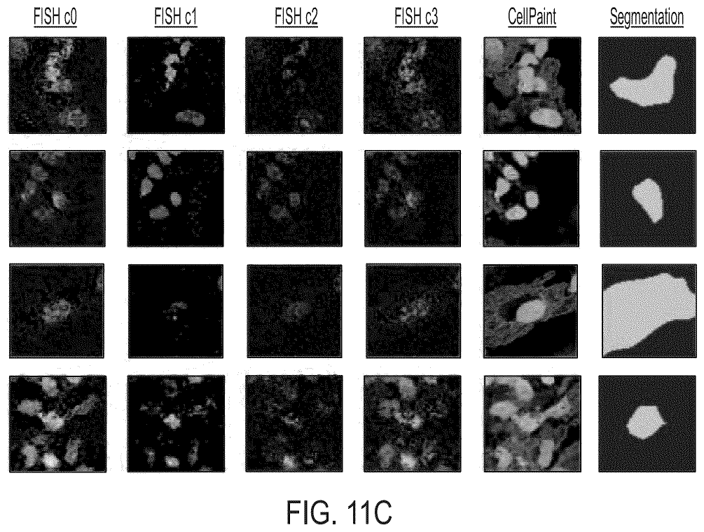

FIG. 9 (split into three color channels as FIGS. 11A-11C) illustrates exemplary images of various readouts of the same cell obtained across four cycles of FISH (e.g., FISHc0-FISHc3), staining with CellPaint, and corresponding cell segmentation to define the boundaries of the individual cells in the images. These measurements can then be used for high-throughput screening of genetic perturbations and small-molecules for gene expression changes. For example, the exemplary measurements obtained in FIG. 9 (and FIGS. 11A-11C) may be used to generate FIG. 10, which illustrates exemplary results of a high-throughput screening of genetic perturbations performed using FISH followed by in situ sequencing. The results shown in FIG. 10 demonstrate high concordance with similar screens performed using Perturb-Seq and FISH-Flow methods (data not shown), thereby illustrating the utility of the methods described herein.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application. While certain embodiments of the present application have been shown and described herein, it will be obvious that such embodiments are provided by way of example only. Numerous variations, changes, and substitutions may occur to those skilled in the art without departing from the spirit and scope of the invention. It should be understood that various alternatives to the embodiments described herein may be employed in practicing the methods described herein.

### Example 1. Exemplary Screening Platform to Analyzing Genetic Perturbations in Barcoded Cells

This example demonstrates use of a screening platform for investigating: i) the effects of genetic perturbations, ii) transcriptional profiling, and iii) morphological/proteomic analysis within a sample, of an exemplary disease.

FIG. 5 illustrates an exemplary schematic of a screening platform. Induced pluripotent stem cells (iPSCs) expressing Cas9 are differentiated. At 502, the differentiated iPSCs are treated with a gRNA library via a lentivirus comprising padlock probe hybridization sequence-flanked gRNAs, enabling the detection of these gRNA barcode sequences via in situ sequencing-by-synthesis. The delivered gRNA binds to the Cas9, producing double-stranded breaks at the locations specified by the gRNA, ultimately leading to a plurality of genetically engineered cells; each of which has had a gene knocked out while expressing padlock probe hybridization sequence-flanked gRNAs 504 that act as a ‘barcode’ specifying the edited gene. The native mRNA 506 of the cells may be analyzed to identify phenotypes associated with the genetic perturbations.

At 508, cells are fixed, and optionally stored at −20° C. in ethanol (EtOH) until needed. Cells then undergo hybridization with 3′ loop probes and 5′ probes (e.g., SNAIL probes), targeting a set of mRNA transcripts. The 3′ loop probes comprise a gene I.D. (GID) sequence that identifies the mRNA transcript to which they hybridize. The mRNA transcripts may have been previously identified as possible biomarkers for an exemplary disease. The probe mixes are then washed, and T4 DNA ligation is conducted to close the loop generated by the 3′ loop probe. The 3′ loop probe is then amplified via rolling circle amplification (RCA), which produces several copies of the GID sequence at the location of each transcript, and the resulting DNA amplicons are fixed at their location via bis-MHS-PEG exposure. Samples are treated with a reverse transcription primer simultaneously with a paraformaldehyde (PFA) and glutaraldehyde fix. Samples are then reverse transcribed overnight, thereby generating reverse transcribed barcode sequences, followed by an additional fix with PFA and glutaraldehyde.

At 510, imaging of the DNA amplicons is done in cycles in order to detect the amplicons. For each cycle, adapter oligonucleotides are hybridized to the GID sequences and to detection probes (e.g., fluorescently labeled probes), which are designed such that each mRNA transcript to be imaged has a single adapter oligonucleotide (e.g., GID) and single detection probe (e.g., fluorophore that binds to it, enabling multiple colors (and thus genes) to be imaged at a time. Samples are then imaged at 20× at 3 z locations, using a confocal microscope to enable accurate quantification of individual RNA ‘dots’. Next, to complete the cycle, toehold displacement oligonucleotides are rinsed over the sample to release the bound adapter oligonucleotide/detection probe pairs from their GID targets, clearing the sample for the next round of imaging. For example, 4 genes may be imaged per cycle, with the 488 channel being used for GAPDH housekeeping during each cycle. After ˜4 cycles, the needed genes are imaged.

Once imaging of the mRNA transcripts is completed (e.g., in situ fluorescence hybridization, or FISH), the sample undergoes processing for the pooled optical screening (e.g., POSH) of the genetically barcoded cells. Briefly, the samples are contacted with padlock probes, which hybridize to the padlock probe hybridization sequences flanking the gRNA target sequences. The samples undergo gap filling, which closes the loop generated by the padlock probes flanking the gRNAs in each cell. The gRNA sequences (e.g., barcode sequences) are then amplified via RCA to produce several copies of the gRNA sequence (e.g., barcode amplicons) to amplify the signal that will be read out during sequencing-by-synthesis.

Optionally, at 512, cells may be stained with “CellPaint,” which is a combination of cellular dyes and antibodies. These stains are also used to segment individual cells, allowing us to determine the boundaries of each cell within the pool. This segmentation allows FISH/SNAIL and POSH dots to be accurately assigned to individual cells.

Once the transcriptional and morphological/proteomic datasets are generated, the gRNA belonging to each cell (e.g., barcode sequences) are detected at 514. This is done by conducting in situ sequencing-by-synthesis on the cells while still in the same culture dish. This is also done in a cyclic manner. Briefly, cells are treated with fluorescently labeled nucleobases, which bind to the amplified gRNA sequences (e.g., barcode amplicons) within each cell. The ‘dots’ within that cell thus fluoresce depending on the open base within the gRNA sequence. The fluorescent base is then cleaved using a stripping buffer. Once again, the fluorescently labeled nucleobases are applied, causing the next base in the sequence to fluoresce based on the gRNA that the cell received. This may be continued for about 14 cycles, which enables the readout of 14 bases for each gRNA that is expressed in the plurality of cells. This is enough cycles to accurately predict which gRNA is present in each cell, allowing a readout of which genetic perturbation each cell received. After this step, within the imaging assay, each cell can be assigned a transcriptome (e.g., based on FISH imaging cycles), and morphology/proteome (e.g., based on CellPaint imaging), and a perturbation readout (e.g., based on POSH sequencing results).

### Example 2. Unified Workflow of FISH and POSH

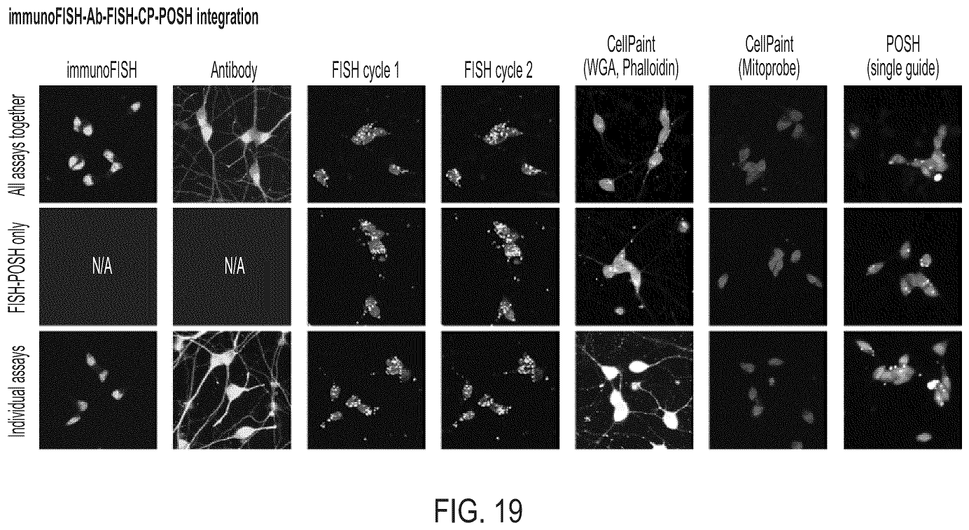

An A549 cell line and an iPSC-derived motor neuron cell line were lentivirally transduced with a single padlock-flanked CRISPR guide RNA comprising a barcode sequence. The gRNA bound to Cas9 and knocked out a single gene in each cell line, resulting in a single-gene-knockout A549 cell line and a single-gene-knockout motor neuron cell line. Cells were fixed and processed using the “Exemplary immunoFISH/FISH/POSH method(I)” described in the Methods of the Assay section of the present disclosure. For the immunoFISH readout, a polyclonal rabbit anti-human primary antibody targeting the TDP43 C-terminal end was conjugated to a single-stranded 30-nucleotide DNA barcode. Regular antibody staining (i.e., immunohistochemistry without FISH) was also used. For the regular antibody staining readout, a mouse anti-Ki-67 primary antibody was used along with the TDP43 immunoFISH primary. Fluorescent secondary antibodies against mouse and rabbit were used. For the RNA FISH readout, FISH probes targeting GAPDH RNA transcripts were used. RNA FISH and immunoFISH fluorescent readouts were both performed in the 555 nm fluorescent range to demonstrate that the RNA FISH signal was detectable after immunoFISH stripping. In the phenotyping step after POSH rolling circle amplification, the cells were stained with a modified version of CellPaint consisting of Mitroprobe, Phalloidin, and Wheat Germ Agglutinin (WGA) without any components in the 488 fluorescent range. The fix used in step (12) of the exemplary method was found to significantly increase the number of POSH amplicons per cell. Prior to this experiment, the method was employed without the fixation of step (12). The addition of the fixation to step (12) approximately doubled the median and mean amplicons per cell (data not shown). In addition, the extra hybridization time of step (12) was found to further increase the median and mean amplicons per cell, increasing both the number of POSH dots and the intensity of those dots (data not shown). The results of the screen for the A549 cell line are shown in FIG. 18 and the results of the screen for the motor neuron cell line are shown in FIG. 19. In FIGS. 18 and 19, the top rows show the unified workflow where the same plurality of cells were assessed in by the listed assays in sequence. The middle rows show the unified workflow including only RNA FISH and POSH. The bottom rows show the indicated assays performed individually on a plurality of cells, that is, not part of the unified workflow.

1. A method, comprising:
(a) providing a plurality of cells, wherein the plurality of cells comprise at least one cell comprising at least one genetic perturbation, wherein said at least one cell comprising the at least one genetic perturbation comprises a barcode sequence associated with the genetic perturbation;
(b) performing at least one round of fluorescence in situ hybridization (FISH);
(c) performing pooled optical screening in human cells (POSH), comprising amplifying the barcode sequence in the at least one cell and sequencing the barcode sequence in situ.

2. The method of claim 1, further comprising analyzing the phenotype of the at least one cell;
wherein analyzing the phenotype comprises at least one assay selected from the group consisting of label-free imaging, high content imaging, calcium imaging, immunohistochemistry, cell morphology imaging, protein aggregation imaging, cell-cell interaction imaging, live cell imaging, and any other imaging-based assay modality.

3-6. (canceled)

7. The method of claim 1, wherein the at least one cell comprises a CRISPR system.

8-12. (canceled)

13. The method of claim 1, wherein the at least one cell has been contacted with a gRNA to generate the at least one perturbation.

14-16. (canceled)

17. The method of claim 1, further comprising contacting the plurality of cells with a gRNA library comprising a plurality of different gRNAs to generate a plurality of genetic perturbations comprising the at least one genetic perturbation.

18-24. (canceled)

25. The method of claim 1, further comprising fixing the plurality of cells on a surface prior to step (b).

26. (canceled)

27. The method of claim 1, further comprising permeabilizing the plurality of cells.

28-29. (canceled)

30. The method of claim 1, wherein the at least one round of FISH comprises at least one round of RNA FISH, wherein each round of RNA FISH is uniquely associated with at least one mRNA transcript from the at least one cell, wherein the at least one round of RNA FISH comprises:
(i) contacting a plurality of mRNA transcripts comprising the at least one mRNA transcript with a plurality of 3′ loop probes, wherein each 3′ loop probe comprises a first target hybridization sequence complementary to a first portion of an mRNA transcript of the plurality of mRNA transcripts;
(ii) contacting the plurality of mRNA transcripts comprising the at least one mRNA transcript with a plurality of 5′ probes, wherein each 5′ probe comprises a second target hybridization sequence complementary to a second portion of the mRNA transcript of the plurality of mRNA transcripts, wherein each 5′ probe is capable of specifically hybridizing with a 3′ loop probe, wherein hybridization of a 5′ probe with a corresponding 3′ loop probe forms a loop in the 3′ loop probe;
(iii) connecting the ends of the loop in each 3′ loop probe to form a plurality of circular probes;
(iv) amplifying a plurality of target sequences using the circular probes as templates to form a plurality of DNA amplicons; and
(v) detecting the DNA amplicons by the at least one round of RNA FISH.

31. (canceled)

32. The method of 1, wherein the at least one round of FISH comprises at least one round of RNA FISH, wherein each round of RNA FISH is uniquely associated with at least one mRNA transcript from the at least one cell, wherein the at least one round of RNA FISH comprises:
(i) contacting a plurality of mRNA transcripts comprising the at least one mRNA transcript with a plurality of 5′ loop probes, wherein each 5′ loop probe comprises a first target hybridization sequence complementary to a first portion of an mRNA transcript of the plurality of mRNA transcripts;
(ii) contacting the plurality of mRNA transcripts comprising the at least one mRNA transcript with a plurality of 3′ probes, wherein each 3′ probe comprises a second target hybridization sequence complementary to a second portion of the mRNA transcript of the plurality of mRNA transcripts, wherein each 3′ probe is capable of specifically hybridizing with a 5′ loop probe, wherein hybridization of a 3′ probe with a corresponding 5′ loop probe forms a loop in the 5′ loop probe;
(iii) connecting the ends of the loop in each 5′ loop probe to form a plurality of circular probes;
(iv) amplifying a plurality of target sequences using the circular probes as templates to form a plurality of DNA amplicons; and
(v) detecting the DNA amplicons by the at least one round of RNA FISH.

33. (canceled)

34. The method of claim 30, wherein detecting the DNA amplicons comprises at least one of:
(I) labeling the DNA amplicons with a fluorophore, an isotope, a mass tag, or a combination thereof;
(II) hybridizing an adapter oligonucleotide to the DNA amplicon;
(III) hybridizing a detection probe to the adapter oligonucleotide,
(IV) hybridizing a detection probe to the adapter oligonucleotide, wherein the detection probe comprises a fluorophore, an isotope, a mass tag, an oligonucleotide, or a combination thereof; or
(V) imaging the DNA amplicons.

35-45. (canceled)

46. The method of claim 30, wherein the DNA amplicon is formed using rolling circle amplification (RCA).

47-56. (canceled)

57. The method of claim 1, wherein the at least one round of FISH comprises at least one round of RNA foci FISH comprising:
(i) contacting RNA foci with a fluorescent oligonucleotide probe, wherein each fluorescent oligonucleotide probe comprises a target hybridization sequence complementary to a portion of a sequence in the RNA foci; and
(ii) detecting the fluorescent oligonucleotide probe by imaging.

58-60. (canceled)

61. The method of claim 1, wherein POSH comprises:
(A) reverse transcribing the barcode sequence to form a reverse transcribed barcode sequence;
(B) hybridizing at least one padlock probe to the reverse transcribed barcode sequence, wherein:
the at least one padlock probe comprises a first barcode hybridization sequence and a second barcode hybridization sequence,
the reverse transcribed barcode sequence comprises a first padlock probe hybridization sequence and a second padlock probe hybridization sequence flanking a target sequence, and
the first barcode hybridization sequence hybridizes with first padlock probe hybridization sequence and the second barcode hybridization sequence hybridizes with second padlock probe hybridization sequence; and
(C) connecting the ends of the at least one padlock probe to form a circular probe.

62. The method of claim 61, further comprising (D) forming a barcode amplicon using the circular probe as a template, wherein the barcode amplicon comprises a plurality of copies of the barcode sequence.

63-76. (canceled)

77. The method of claim 61, further comprising fixing the reverse transcribed barcode sequence in place after step (A) of POSH.

78. The method of claim 77, wherein fixing the reverse transcribed barcode sequence comprises at least one of paraformaldehyde (PFA) or glutaraldehyde treatment.

79. (canceled)

80. The method of claim 77, wherein fixing the reverse transcribed barcode sequence is performed after step (iv) of the at least one round of RNA FISH.

81. (canceled)

82. The method of claim 62, wherein the barcode amplicon is formed after step (v) of the at least one round of RNA FISH.

83-84. (canceled)

85. The method of claim 57, wherein the at least one round of RNA foci FISH is performed before step (A) of POSH.

86. The method of claim 57, wherein step (i) of contacting RNA foci with the fluorescent oligonucleotide probe is carried out during an incubation period at a temperature of at least 50° C.

87. The method of claim 1, wherein the at least one round of FISH comprises at least one round of immunoFISH, wherein each round of immunoFISH is uniquely associated with at least one protein of interest from the at least one cell, wherein the at least one round of immunoFISH comprises:
(i) contacting the at least one cell with a conjugate, the conjugate comprising an antibody which specifically binds the at least one protein of interest and a DNA oligonucleotide comprising a protein identification sequence uniquely associated with the protein of interest;
(ii) contacting the conjugate with an adapter oligonucleotide, wherein the adapter oligonucleotide comprises a first adapter sequence complementary to a portion of the DNA oligonucleotide;
(iii) contacting the adapter oligonucleotide with a fluorescent oligonucleotide capable of hybridizing with a second adapter sequence of the adapter oligonucleotide;
(iv) imaging the at least one cell to detect the at least one fluorescent oligonucleotide.

88. (canceled)

89. The method of claim 1, the at least one round of FISH comprises at least one round of immunoFISH, wherein each round of immunoFISH is uniquely associated with at least one protein of interest from the at least one cell, wherein the at least one round of immunoFISH comprises:
(i) contacting the at least one cell with a first antibody which specifically binds the at least one protein of interest;
(ii) contacting the antibody with a conjugate comprising an antibody which specifically binds the first antibody and a DNA oligonucleotide comprising a protein identification sequence uniquely associated with the protein of interest;
(iii) contacting the conjugate with an adapter oligonucleotide, wherein the adapter oligonucleotide comprises a first adapter sequence complementary to a portion of the DNA oligonucleotide;
(iv) contacting the adapter oligonucleotide with a fluorescent oligonucleotide capable of hybridizing with a second adapter sequence of the adapter oligonucleotide;
(v) imaging the at least one cell to detect the at least one fluorescent oligonucleotide.

90-96. (canceled)

97. The method of claim 2, wherein analyzing the phenotype of the at least one cell is performed before segmenting a morphological image of the cell, wherein segmenting comprises detecting the cell and identifying the boundaries of the cell in the morphological image.

98. (canceled)

99. The method of claim 97, further comprising processing and/or transforming the morphological image to obtain images of the same cell with different readouts.

100. The method of claim 99, wherein the images of the same cell are obtained from the at least one round of FISH, sequencing of the barcode sequence in situ, and/or segmenting of the morphological image of the cell.

101. The method of claim 1, further comprising:
receiving a first image depicting the plurality of cells, wherein the first image indicates each cell of the plurality of cells by a boundary and associates each cell of the plurality of cells with a corresponding cell identifier;
receiving a second image depicting locations of a plurality of barcode sequences, wherein the plurality of barcode sequences are associated with the plurality of cells after POSH is performed;
aligning the first image and the second image;
based on the alignment of the first image and the second image, identifying an association between each cell of the plurality of cells and a corresponding barcode sequence of the plurality of barcode sequences;
receiving a FISH image of the plurality of cells after the at least one round of FISH is performed on the plurality of cells;
aligning the first image and the FISH image;
based on the alignment between the first image and the FISH image, resizing the FISH image;
associating each cell of the plurality of cells with a portion of the resized FISH image, the corresponding barcode sequence, and the corresponding cell identifier; and
analyzing known phenotypes or identifying new phenotypes of the plurality of cells.

102-116. (canceled)

117. A method for analyzing known phenotypes or identifying new phenotypes of a plurality of cells, comprising:
receiving a first image depicting the plurality of cells, wherein the first image indicates each cell of the plurality of cells by a boundary and associates each cell of the plurality of cells with a corresponding cell identifier;
receiving a second image depicting locations of a plurality of barcode sequences, wherein the plurality of barcode sequences are associated with the plurality of cells after one or more in situ sequencing cycles are performed;
aligning the first image and the second image;
based on the alignment of the first image and the second image, identifying an association between each cell of the plurality of cells and a corresponding barcode sequence of the plurality of barcode sequences;
receiving a FISH image of the plurality of cells after a FISH cycle is performed on the plurality of cells;
aligning the first image and the FISH image;
based on the alignment between the first image and the FISH image, resizing the FISH image;
associating each cell of the plurality of cells with a portion of the resized FISH image, the corresponding barcode sequence, and the corresponding cell identifier; and
analyzing known phenotypes or identifying new phenotypes of the plurality of cells.

118-144. (canceled)

145. A system for analyzing known phenotypes or identifying new phenotypes of a plurality of cells, comprising:
one or more processors;
a memory; and
one or more programs, wherein the one or more programs are stored in the memory and configured to be executed by the one or more processors, the one or more programs including instructions for:
receiving a first image depicting the plurality of cells, wherein the first image indicates each cell of the plurality of cells by a boundary and associates each cell of the plurality of cells with a corresponding cell identifier;
receiving a second image depicting locations of a plurality of barcode sequences, wherein the plurality of barcode sequences are associated with the plurality of cells after one or more in situ sequencing cycles are performed;
aligning the first image and the second image;
based on the alignment of the first image and the second image, identifying an association between each cell of the plurality of cells and a corresponding barcode sequence of the plurality of barcode sequences;
receiving a FISH image of the plurality of cells after a FISH cycle is performed on the plurality of cells;
aligning the first image and the FISH image;
based on the alignment between the first image and the FISH image, resizing the FISH image;
associating each cell of the plurality of cells with a portion of the resized FISH image, the corresponding barcode sequence, and the corresponding cell identifier; and
analyzing known phenotypes or identifying new phenotypes of the plurality of cells.

146. (canceled)
